SEQUENCE LISTING (120 sequences)

SEQ ID NO: 1 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - protein, 176 aa, synthetic construct
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 3 - protein, 178 aa, synthetic construct
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 4 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 5 - protein, 10 aa, Homo sapiens
SVLNYERARR

SEQ ID NO: 6 - protein, 6 aa, synthetic construct
DSASNY

SEQ ID NO: 7 - protein, 7 aa, synthetic construct
IRSNVGE

SEQ ID NO: 8 - protein, 9 aa, synthetic construct
CADWVDMRF

SEQ ID NO: 9 - protein, 128 aa, synthetic construct
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCADWVDMRFGAGTRLTVKPN

SEQ ID NO: 10 - protein, 268 aa, synthetic construct
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCADWVDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 11 - protein, 6 aa, synthetic construct
DFQATT

SEQ ID NO: 12 - protein, 7 aa, synthetic construct
SNEGSKA

SEQ ID NO: 13 - protein, 15 aa, synthetic construct
CSAPLDRGSNQPQHF

SEQ ID NO: 14 - protein, 143 aa, synthetic construct
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAPLDRGSNQPQHFGDGTRLSILE

SEQ ID NO: 15 - protein, 319 aa, synthetic construct
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAPLDRGSNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 16 - protein, 321 aa, synthetic construct
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAPLDRGSNQPQHFGDGTRLSILEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 17 - protein, 6 aa, synthetic construct
TSGFNG

SEQ ID NO: 18 - protein, 6 aa, synthetic construct
NVLDGL

SEQ ID NO: 19 - protein, 15 aa, synthetic construct
CAVSRPNSGYSTLTF

SEQ ID NO: 20 - protein, 130 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVSRPNSGYSTLTFGKGTMLLVSPD

SEQ ID NO: 21 - protein, 270 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVSRPNSGYSTLTFGKGTMLLVSPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 5 aa, synthetic construct
PRHDT

SEQ ID NO: 23 - protein, 6 aa, synthetic construct
FYEKMQ

SEQ ID NO: 24 - protein, 16 aa, synthetic construct
CASSVRTPSGQETQYF

SEQ ID NO: 25 - protein, 145 aa, synthetic construct
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSVRTPSGQETQYFGPGTRLLVLE

SEQ ID NO: 26 - protein, 320 aa, synthetic construct
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSVRTPSGQETQYFGPGTRLLVLDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 27 - protein, 323 aa, synthetic construct
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSVRTPSGQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 28 - protein, 6 aa, synthetic construct
VSNAYN

SEQ ID NO: 29 - protein, 4 aa, synthetic construct
GSKP

SEQ ID NO: 30 - protein, 15 aa, synthetic construct
CAVETGGGATNKLIF

SEQ ID NO: 31 - protein, 134 aa, synthetic construct
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVETGGGATNKLIFGTGTLLAVQPN

SEQ ID NO: 32 - protein, 274 aa, synthetic construct
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVETGGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 33 - protein, 5 aa, synthetic construct
MNHNS

SEQ ID NO: 34 - protein, 6 aa, synthetic construct
SASEGT

SEQ ID NO: 35 - protein, 15 aa, synthetic construct
CASSEFWLTQETQYF

SEQ ID NO: 36 - protein, 134 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEFWLTQETQYFGPGTRLLVLE

SEQ ID NO: 37 - protein, 310 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEFWLTQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 38 - protein, 312 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSEFWLTQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 39 - protein, 5 aa, synthetic construct
DSVNN

SEQ ID NO: 40 - protein, 5 aa, synthetic construct
IPSGT

SEQ ID NO: 41 - protein, 16 aa, synthetic construct
CAVSHGRGGATNKLIF

SEQ ID NO: 42 - protein, 133 aa, synthetic construct
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVSHGRGGATNKLIFGTGTLLAVQPN

SEQ ID NO: 43 - protein, 273 aa, synthetic construct
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVSHGRGGATNKLIFGTGTLLAVQPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 5 aa, synthetic construct
MNHNS

SEQ ID NO: 45 - protein, 6 aa, synthetic construct
SASEGT

SEQ ID NO: 46 - protein, 13 aa, synthetic construct
CASDRVLGYEQYF

SEQ ID NO: 47 - protein, 132 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASDRVLGYEQYFGPGTRLTVTE

SEQ ID NO: 48 - protein, 308 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASDRVLGYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 49 - protein, 309 aa, synthetic construct
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASDRVLGYEQYFGPGTRLTVTDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 50 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 51 - protein, 9 aa, Homo sapiens
LMLGEFLKL

SEQ ID NO: 52 - protein, 6 aa, synthetic construct
NYSPAY

SEQ ID NO: 53 - protein, 7 aa, synthetic construct
IRENEKE

SEQ ID NO: 54 - protein, 14 aa, synthetic construct
CALDRMDSSYKLIF

SEQ ID NO: 55 - protein, 134 aa, synthetic construct
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDRMDSSYKLIFGSGTRLLVRPD

SEQ ID NO: 56 - protein, 274 aa, synthetic construct
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDRMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 57 - protein, 5 aa, synthetic construct
MNHEY

SEQ ID NO: 58 - protein, 6 aa, synthetic construct
SVGAGI

SEQ ID NO: 59 - protein, 13 aa, synthetic construct
CASSYDQDGEAFF

SEQ ID NO: 60 - protein, 132 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYDQDGEAFFGQGTRLTVVE

SEQ ID NO: 61 - protein, 308 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYDQDGEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 62 - protein, 310 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYDQDGEAFFGQGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 63 - DNA, 807 bp, synthetic construct
  source 1..807: mol_type = other DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagactgggttgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 64 - DNA, 960 bp, synthetic construct
  source 1..960: mol_type = other DNA
atgctgctgcttctgctgcttctggggccaggtataagcctccttctacctgggagcttggcaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctcctctcgaccgggggagcaatcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 65 - DNA, 966 bp, synthetic construct
  source 1..966: mol_type = other DNA
atgctgctgcttctgctgcttctggggccaggtataagcctccttctacctgggagcttggcaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctcctctcgaccgggggagcaatcagccccagcattttggtgatgggactcgactctccatcctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaa

SEQ ID NO: 66 - DNA, 807 bp, synthetic construct
  source 1..807: mol_type = other DNA
atgaccagcatcagagccgtgttcatcttcctgtggctgcagctggacctggtcaacggcgagaatgtggaacagcaccccagcacactgagcgtgcaagagggcgattctgccgtgatcaagtgcacctacagcgacagcgccagcaactacttcccctggtacaagcaagagctgggcaaaagaccccagctgatcatcgacatccggtccaacgtgggcgagaagaaggaccagagaatcgccgtgacactgaacaagaccgccaagcacttcagcctgcacatcaccgagacacagcctgaggatagcgccgtgtacttctgcgccgattgggtcgacatgagattcggagccggcaccagactgaccgtgaagcccaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcacagataagtgcgtgctggacatgcggagcatggacttcaagagcaacagcgccgtggcctggtccaacaagtccgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgctgaaagtggccggcttcaacctgctgatgaccctgagactgtggtcctcctga

SEQ ID NO: 67 - DNA, 966 bp, synthetic construct
  source 1..966: mol_type = other DNA
atgttgcttttgctgcttctgctcggccctggcatctctttgctgctgcctggttctctggccggctctggacttggagctgtggtgtctcagcacccctcttgggtcatctgcaagagcggcaccagcgtgaagatcgagtgcagaagcctggacttccaggccaccaccatgttctggtacagacagttccccaagcagagcctgatgctgatggccacctctaacgagggcagcaaggccacatatgagcagggcgtcgagaaggacaagttcctgatcaaccacgccagcctgacactgagcaccctgacagtgacaagcgcccatcctgaggacagcagcttctacatctgtagcgcccctctggaccggggctctaatcagcctcagcactttggcgacggcaccagactgagcatcctggaagatctgaagaacgtgttcccacctgaggtggccgtgttcgagccttctgaggccgagatcagccacacacagaaagccacactcgtgtgtctggccaccggcttctatcccgatcacgtcgaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcaggtgccaggtgcagttctacggcctgagcgagaacgatgagtggacccaggatagagccaagcctgtgacacagatcgtgtctgccgaagcctggggcagagccgattgtggctttaccagcgagagctaccagcaaggcgtgctgtctgccaccatcctgtacgagatcctgctgggcaaagccactctgtacgccgtgctggtgtctgccctggtcctgatggctatggtcaagagaaaggacagccggggctga

SEQ ID NO: 68 - DNA, 813 bp, synthetic construct
  source 1..813: mol_type = other DNA
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagtaggccgaattcaggatacagcaccctcacctttgggaaggggactatgcttctagtctctccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 69 - DNA, 966 bp, synthetic construct
  source 1..966: mol_type = other DNA
atgcttagtcctgacctgcctgactctgcctggaacaccaggctcctctgccatgtcatgctttgtctcctgggagcagtttcagtggctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagtgtcaggactccgtcggggcaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 70 - DNA, 972 bp, synthetic construct
  source 1..972: mol_type = other DNA
atgcttagtcctgacctgcctgactctgcctggaacaccaggctcctctgccatgtcatgctttgtctcctgggagcagtttcagtggctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagtgtcaggactccgtcggggcaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaa

SEQ ID NO: 71 - DNA, 813 bp, synthetic construct
  source 1..813: mol_type = other DNA
atgtggggagtgtttctgctgtacgtgtccatgaagatgggcggcaccaccggccagaacatcgatcagcctacagagatgaccgccaccgagggcgccatcgtgcagatcaattgcacctaccagaccagcggcttcaacggcctgttctggtatcagcagcatgccggcgaggcccctaccttcctgagctacaatgtgctggacggcctggaagagaagggcagattcagcagcttcctgtccagaagcaagggctacagctacctgctgctgaaagaactgcagatgaaggacagcgcctcctacctgtgcgccgtgtccagacctaatagcggctacagcaccctgaccttcggcaagggaacaatgctgctggtgtcccctgacattcagaaccccgatcctgccgtgtaccagctgagagacagcaagagcagcgacaagagcgtgtgcctgttcaccgacttcgacagccagaccaacgtgtcccagagcaaggactccgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgcttaaggtggccggcttcaatctgctgatgaccctgagactgtggtccagctga

SEQ ID NO: 72 - DNA, 972 bp, synthetic construct
  source 1..972: mol_type = other DNA
atgctttctcctgatcttcctgacagcgcctggaacaccagactgctgtgtcacgtgatgctgtgtctgctgggagccgtgtctgttgccgctggcgttatccagtctcctcggcacctgatcaaagagaagagagagacagccacactgaagtgctaccccattccacggcacgacaccgtgtactggtatcagcaaggcccaggccaggatcctcagttcctgatcagcttctacgagaagatgcagagcgacaagggcagcatccccgacagattttctgcccagcagttcagcgactaccacagcgagctgaacatgagcagcctggaactgggcgatagcgccctgtacttttgtgccagctctgtgcggacacccagcggccaagaaacccagtattttggccccggaacacggctgctggtgctggaagatctgaagaacgtgttcccacctgaggtggccgtgttcgagccttctgaggccgagatctctcacacccagaaagccacactcgtgtgtctggccaccggcttctatcccgatcacgtggaactgtcttggtgggtcaacggcaaagaggtgcacagcggcgtctgtaccgatcctcagcctctgaaagaacagcccgctctgaacgacagccggtactgtctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggaaaagccactctgtacgccgtgctggtgtctgccctggtgctgatggccatggtcaagcggaaggatagcaggggctaa

SEQ ID NO: 73 - DNA, 825 bp, synthetic construct
  source 1..825: mol_type = other DNA
atggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtggagactgggggtggtgctacaaacaagctcatctttggaactggcactctgcttgctgtccagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 74 - DNA, 933 bp, synthetic construct
  source 1..933: mol_type = other DNA
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgaattttggcttacccaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 75 - DNA, 939 bp, synthetic construct
  source 1..939: mol_type = other DNA
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgaattttggcttacccaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaa

SEQ ID NO: 76 - DNA, 825 bp, synthetic construct
  source 1..825: mol_type = other DNA
atggctctgcagtctacactgggcgctgtttggctgggcctgctgctgaattctctgtggaaggtggccgagagcaaggaccaggtgttccagccttctacagtggcctcttctgagggcgccgtggtggaaatcttctgcaaccacagcgtgtccaacgcctacaacttcttctggtatctgcacttccccggctgcgcccctagactgcttgtgaagggaagcaagcctagccagcagggcagatacaacatgacctacgagcggttcagcagctccctgctgattctgcaagtgcgggaagccgatgccgccgtgtattactgtgctgtggaaacaggcggcggagccaccaacaagctgatctttggaaccggcacactgctggccgtgcagcccaatattcagaaccctgatcctgccgtgtaccagctgagagacagcaagagcagcgacaagagcgtgtgcctgttcaccgacttcgacagccagaccaacgtgtcccagtccaaggacagcgacgtgtacatcaccgataagtgcgtgctggacatgcggagcatggacttcaagagcaacagcgccgtggcctggtccaacaagagcgatttcgcctgcgccaatgccttcaacaacagcattatccccgaggacacattcttcccaagtcctgagagcagctgcgacgtgaagctggtggaaaagagcttcgagacagacaccaacctgaacttccagaacctgagcgtgatcggcttcaggatcctcctgctgaaagtggccggcttcaacctgctgatgaccctgagactgtggtccagctga

SEQ ID NO: 77 - DNA, 939 bp, synthetic construct
  source 1..939: mol_type = other DNA
atgtctattggactgctgtgctgcgtggccttcagcctgctttgggcctctcctgtgaatgccggcgtgacccagacacctaagttccaggtgctgaaaaccggccagagcatgaccctgcagtgcgcccaggacatgaaccacaacagcatgtactggtacagacaggaccccggcatgggcctgagactgatctactactctgccagcgagggcaccaccgacaaaggcgaagtgcccaatggctacaacgtgtcccggctgaacaagagagagttcagcctgaggctggaaagcgccgctccatctcagaccagcgtgtacttttgtgccagcagcgagttctggctgacccaagagacacagtacttcggccctggcaccagactgctggtcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggctaa

SEQ ID NO: 78 - DNA, 822 bp, synthetic construct
  source 1..822: mol_type = other DNA
atgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtgtcccacggaaggggtggtgctacaaacaagctcatctttggaactggcactctgcttgctgtccagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 79 - DNA, 927 bp, synthetic construct
  source 1..927: mol_type = other DNA
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcgacagggtattagggtacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 80 - DNA, 933 bp, synthetic construct
  source 1..933: mol_type = other DNA
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcgacagggtattagggtacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaa

SEQ ID NO: 81 - DNA, 822 bp, synthetic construct
  source 1..822: mol_type = other DNA
atgaagagaatcctgggcgccctgctgggactgctgtctgcccaagtctgttgtgtgcggggcatccaggtggaacagagccctcctgatctgatcctgcaagagggcgccaacagcaccctgagatgcaacttcagcgacagcgtgaacaacctgcagtggttccaccagaatccttggggccagctgatcaacctgttctacatccccagcggcaccaagcagaacggcagactgtctgctaccaccgtggccaccgagagatacagcctgctgtacatcagcagcagccagaccacagacagcggcgtgtacttctgtgccgtgtctcatggaagaggcggagccaccaacaagctgatctttggcacaggcacactgctggccgtgcagcccaatattcagaaccctgatccagccgtgtaccagctgagagacagcaagagcagcgacaagagcgtgtgtctgttcaccgacttcgactcccagaccaacgtgtcccagagcaaggactccgacgtgtacatcaccgataagtgcgtgctggacatgcggagcatggacttcaagagcaacagcgccgtggcctggtccaacaagagcgattttgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggatcctgctgcttaaggtggccggcttcaacctgctgatgaccctgagactgtggtccagctga

SEQ ID NO: 82 - DNA, 933 bp, synthetic construct
  source 1..933: mol_type = other DNA
atgtctattggactgctgtgctgcgtggccttcagcctgctttgggcctctcctgtgaatgccggcgtgacccagacacctaagttccaggtgctgaaaaccggccagagcatgaccctgcagtgcgcccaggacatgaaccacaacagcatgtactggtacagacaggaccccggcatgggcctgagactgatctactactctgccagcgagggcaccaccgacaaaggcgaagtgcccaatggctacaacgtgtcccggctgaacaagagagagttcagcctgaggctggaaagcgccgctccatctcagaccagcgtgtacttctgcgccagcgatagagtgctgggctacgagcagtatttcggccctggcaccagactgaccgtgaccgaggatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggctaa

SEQ ID NO: 83 - DNA, 825 bp, synthetic construct
  source 1..825: mol_type = other DNA
atggagtcattcctgggaggtgttttgctgattttgtggcttcaagtggactgggtgaagagccaaaagatagaacagaattccgaggccctgaacattcaggagggtaaaacggccaccctgacctgcaactatacaaactattccccagcatacttacagtggtaccgacaagatccaggaagaggccctgttttcttgctactcatacgtgaaaatgagaaagaaaaaaggaaagaaagactgaaggtcacctttgataccacccttaaacagagtttgtttcatatcacagcctcccagcctgcagactcagctacctacctctgtgctctagaccggatggatagcagctataaattgatcttcgggagtgggaccagactgctggtcaggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 84 - DNA, 927 bp, synthetic construct
  source 1..927: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgatcaggacggtgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 85 - DNA, 933 bp, synthetic construct
  source 1..933: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgatcaggacggtgaagctttctttggacaaggcaccagactcacagttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaa

SEQ ID NO: 86 - DNA, 825 bp, synthetic construct
  source 1..825: mol_type = other DNA
atggaatcctttcttggcggcgtgctgctgatcctgtggctgcaagtggattgggtcaagagccagaagatcgagcagaacagcgaggccctgaacatccaagagggcaagaccgccacactgacctgcaactacaccaactacagccccgcctacctgcagtggtacagacaggatccaggcagaggccctgtgttcctgctgctcatccgcgagaacgagaaagagaagcgcaaagaacggctgaaagtgaccttcgacaccacactgaagcagagcctgttccacatcaccgcctctcagcctgccgatagcgccacatatctgtgcgccctggacagaatggacagcagctacaagctgatcttcggcagcggcaccagactgctcgtgcggcccgatattcagaaccctgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggattctgctgctgaaggtggccggcttcaacctgctgatgaccctgagactgtggtccagctga

SEQ ID NO: 87 - DNA, 933 bp, synthetic construct
  source 1..933: mol_type = other DNA
atgtctattggactgctttgttgcgccgctctgagcctgctttgggccggacctgttaatgctggcgtgacccagacacctaagttccaggtgctgaaaaccggccagagcatgaccctgcagtgcgcccaggatatgaaccacgagtacatgagctggtacagacaggaccctggcatgggcctgagactgatccactattctgtcggagccggcatcaccgaccagggcgaagttcctaatggctacaacgtgtccagaagcaccaccgaggacttcccactgagactgctgtctgccgctcctagccagaccagcgtgtacttttgtgccagcagctacgaccaggacggcgaggccttttttggccaaggcaccagactgaccgtggtggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgttctgtctgccaccatcctgtacgagatcctgctgggcaaagccactctgtacgccgtgctggtgtctgccctggtgctgatggccatggtcaagcggaaggatagcaggggctaa

SEQ ID NO: 88 - protein, 9 aa, Homo sapiens
LTLGEFLKL

SEQ ID NO: 89 - RNA, 20 nt, synthetic construct
  source 1..20: mol_type = other RNA
gagtcacattctctatggtc

SEQ ID NO: 90 - RNA, 20 nt, synthetic construct
  source 1..20: mol_type = other RNA
aatgtggcaacgtggtgctc

SEQ ID NO: 91 - RNA, 20 nt, synthetic construct
  source 1..20: mol_type = other RNA
ctaaatggggatttccgcaa

SEQ ID NO: 92 - RNA, 20 nt, synthetic construct
  source 1..20: mol_type = other RNA
atccccatttagccagtatc

SEQ ID NO: 93 - RNA, 20 nt, synthetic construct
  source 1..20: mol_type = other RNA
ctctctgccgagtcggtgca

SEQ ID NO: 94 - RNA, 20 nt, synthetic construct
  source 1..20: mol_type = other RNA
gtgaagtctctctgccgagt

SEQ ID NO: 95 - RNA, 20 nt, synthetic construct
  source 1..20: mol_type = other RNA
tgccccatgcatagttacct

SEQ ID NO: 96 - RNA, 20 nt, synthetic construct
  source 1..20: mol_type = other RNA
aggtcacccctgcaccgact

SEQ ID NO: 97 - RNA, 20 nt, synthetic construct
  source 1..20: mol_type = other RNA
tggcccaggtaactatgcat

SEQ ID NO: 98 - RNA, 20 nt, synthetic construct
  source 1..20: mol_type = other RNA
ccaaggatgcttaccaccag

SEQ ID NO: 99 - RNA, 20 nt, synthetic construct
  source 1..20: mol_type = other RNA
caccgcggcgcggtactcgc

SEQ ID NO: 100 - RNA, 20 nt, synthetic construct
  source 1..20: mol_type = other RNA
gctcagcaccgtgtagcggc

SEQ ID NO: 101 - RNA, 20 nt, synthetic construct
  source 1..20: mol_type = other RNA
accttcgtctgtatgctgtt

SEQ ID NO: 102 - RNA, 20 nt, synthetic construct
  source 1..20: mol_type = other RNA
accaaacagcatacagacga

SEQ ID NO: 103 - RNA, 20 nt, synthetic construct
  source 1..20: mol_type = other RNA
agttgagaaaccccgcctac

SEQ ID NO: 104 - RNA, 20 nt, synthetic construct
  source 1..20: mol_type = other RNA
gttgagaaaccccgcctaca

SEQ ID NO: 105 - RNA, 20 nt, synthetic construct
  source 1..20: mol_type = other RNA
ttgagaaaccccgcctacag

SEQ ID NO: 106 - RNA, 20 nt, synthetic construct
  source 1..20: mol_type = other RNA
tgtagcaccgcccagacgac

SEQ ID NO: 107 - RNA, 20 nt, synthetic construct
  source 1..20: mol_type = other RNA
cgtctgggcggtgctacaac

SEQ ID NO: 108 - RNA, 20 nt, synthetic construct
  source 1..20: mol_type = other RNA
aggtgccgctgtcattgcgc

SEQ ID NO: 109 - RNA, 20 nt, synthetic construct
  source 1..20: mol_type = other RNA
cctgctcgtggtgaccgaag

SEQ ID NO: 110 - RNA, 20 nt, synthetic construct
  source 1..20: mol_type = other RNA
aaggatggctaggatattct

SEQ ID NO: 111 - RNA, 20 nt, synthetic construct
  source 1..20: mol_type = other RNA
aagaatatcctagccatcct

SEQ ID NO: 112 - RNA, 20 nt, synthetic construct
  source 1..20: mol_type = other RNA
gagaatcaaaatcggtgaat

SEQ ID NO: 113 - RNA, 20 nt, synthetic construct
  source 1..20: mol_type = other RNA
tctccgagagcccgtagaac

SEQ ID NO: 114 - RNA, 20 nt, synthetic construct
  source 1..20: mol_type = other RNA
caaacacagcgacctcgggt

SEQ ID NO: 115 - RNA, 20 nt, synthetic construct
  source 1..20: mol_type = other RNA
tgacagcggaagtggttgcg

SEQ ID NO: 116 - RNA, 20 nt, synthetic construct
  source 1..20: mol_type = other RNA
aagtgaagagttggcagaca

SEQ ID NO: 117 - RNA, 20 nt, synthetic construct
  source 1..20: mol_type = other RNA
aactacccctttgacttcca

SEQ ID NO: 118 - RNA, 20 nt, synthetic construct
  source 1..20: mol_type = other RNA
tctgctgggcaaattcagtc

SEQ ID NO: 119 - RNA, 20 nt, synthetic construct
  source 1..20: mol_type = other RNA
ctctcagctggtacacggca

SEQ ID NO: 120 - RNA, 20 nt, synthetic construct
  source 1..20: mol_type = other RNA
acaaaactgtgctagacatg

(19) United States
(12) Patent Application Publication
(10) Pub. No.: US 2026/0232802 A1
(43) Pub. Date: Aug. 13, 2026

(54) T-Cell Receptors

(71) Applicants: Ospedale San Raffaele S.r.l., Milan (IT); Università Vita-Salute San Raffaele, Milan (IT)

(72) Inventors: Maria Chiara Bonini, Milan (IT); Eliana Ruggiero, Milan (IT); Francesco Manfredi, Milan (IT)

(21) Appl. No.: 19/147,356

(22) PCT Filed: Jan. 16, 2024
(86) PCT No.: PCT/EP2024/050933

§ 371 Date: Jul. 11, 2025

(30) Foreign Application Priority Data
Jan. 16, 2023 (EP) 23151852.3

(51) Int. Cl. A61K 40/42; A61K 38/00; A61K 40/11; A61K 40/31; A61K 40/32; A61P 35/00; C07K 14/705; C07K 16/18; C07K 16/40; C12N 5/0783

(52) U.S. Cl. CPC A61K 40/4246; A61K 40/11; A61K 40/31; A61K 40/32; A61P 35/00; C07K 14/7051; C07K 14/70517; C07K 16/18; C07K 16/40; C12N 5/0636; A61K 38/00; C07K 2319/00; C12N 2510/00

(57) ABSTRACT

The present invention provides a T-cell receptor (TCR) which binds to a Human Telomerase Reverse Transcriptase (hTERT) peptide or a Survivin peptide when presented by a major histocompatibility complex (MHC).

### FIELD OF THE INVENTION

The present invention relates to T-cell receptors (TCRs) which bind to immunogenic peptides when presented by a major histocompatibility complex (MHC). The present invention further relates to immunogenic peptides.

### BACKGROUND TO THE INVENTION

The fifth pillar of cancer care is represented by immunotherapy, which exploits the innate ability of the immune system to recognize cancer cells. Indeed, the presence of tumour-infiltrating lymphocytes is correlated with a better prognosis (see e.g. Zhang, L., et al. New England journal of medicine, 348(3), pp. 203-213).

The ability of T lymphocytes to recognise an antigen is determined by the expression of a T cell receptor (TCR). TCR gene therapy is based on the genetic transfer of high-avidity tumour-specific TCR genes into T lymphocytes, thus enabling the specific targeting of the desired tumour-associated antigens and leading to a less toxic and more specific and effective therapy. This approach has shown promise in clinical trials.

One of the main barriers limiting the exploitation of TCR gene therapy for clinical treatment of cancers is the lack of tumour-specific T-cells and corresponding TCRs. Tumour-specific T cells against tumour-associated antigens (TAAs), self-proteins overexpressed on cancer cells, are interesting targets in the context of adoptive T cell therapy. However, the number of isolated TAA-specific T cells is still limited, particularly in the context of blood malignancies.

Thus, the low availability of tumour-specific TCRs still remains an open issue limiting the broad exploitation of TCR-based immunotherapeutic approaches.

### SUMMARY OF THE INVENTION

The present inventors have identified novel TCRs which bind to a Human Telomerase Reverse Transcriptase (hTERT) peptide or a Survivin peptide when presented by a major histocompatibility complex (MHC). The present inventors have determined the amino acid sequences of the TCRs, including the amino acid sequences of their CDR regions, which are responsible for binding specificity for the immunogenic peptides. The TCRs efficiently endowed engineered T cells with tumour-specific cytolytic functions.

In one aspect, the present invention provides a T-cell receptor (TCR) which binds to a Human Telomerase Reverse Transcriptase (hTERT) peptide or a Survivin peptide when presented by a major histocompatibility complex (MHC).

In one aspect, the TCR binds to a Human Telomerase Reverse Transcriptase (hTERT) peptide when presented by an MHC. Suitably, the TCR comprises: (i) a CDR3α comprising the amino acid sequence of CADWVDMRF (SEQ ID NO: 8) or a variant thereof having up to three amino acid substitutions, additions or deletions, and a CDR3β comprising the amino acid sequence of CSAPLDRGSNQPQHF (SEQ ID NO: 13) or a variant thereof having up to three amino acid substitutions, additions or deletions; (ii) a CDR3α comprising the amino acid sequence of CAVSRPNSGYSTLTF (SEQ ID NO: 19) or a variant thereof having up to three amino acid substitutions, additions or deletions, and a CDR3β comprising the amino acid sequence of CASSVRTPSGQETQYF (SEQ ID NO: 24) or a variant thereof having up to three amino acid substitutions, additions or deletions; (iii) a CDR3α comprising the amino acid sequence of CAVETGGGATNKLIF (SEQ ID NO: 30) or a variant thereof having up to three amino acid substitutions, additions or deletions, and a CDR3β comprising the amino acid sequence of CASSEFWLTQETQYF (SEQ ID NO: 35) or a variant thereof having up to three amino acid substitutions, additions or deletions; or (iv) a CDR3α comprising the amino acid sequence of CAVSHGRGGATNKLIF (SEQ ID NO: 41) or a variant thereof having up to three amino acid substitutions, additions or deletions, and a CDR3β comprising the amino acid sequence of CASDRVLGYEQYF (SEQ ID NO: 46) or a variant thereof having up to three amino acid substitutions, additions or deletions. In some embodiments, the TCR comprises the following CDR sequences: (i) CDR1α—DSASNY (SEQ ID NO: 6), CDR2α—IRSNVGE (SEQ ID NO: 7), CDR3α—CADWVDMRF (SEQ ID NO: 8), CDR1β—DFQATT (SEQ ID NO: 11), CDR2β—SNEGSKA (SEQ ID NO: 12), and CDR3β—CSAPLDRGSNQPQHF (SEQ ID NO: 13), or variants thereof each having up to three amino acid substitutions, additions or deletions; (ii) CDR1α—TSGFNG (SEQ ID NO: 17), CDR2α—NVLDGL (SEQ ID NO: 18), CDR3α—CAVSRPNSGYSTLTF (SEQ ID NO: 19), CDR1β—PRHDT (SEQ ID NO: 22), CDR2β—FYEKMQ (SEQ ID NO: 23), and CDR3β—CASSVRTPSGQETQYF (SEQ ID NO: 24), or variants thereof each having up to three amino acid substitutions, additions or deletions; (iii) CDR1α—VSNAYN (SEQ ID NO: 28), CDR2α—GSKP (SEQ ID NO: 29), CDR3α—CAVETGGGATNKLIF (SEQ ID NO: 30), CDR1β —MNHNS (SEQ ID NO: 33), CDR2β—SASEGT (SEQ ID NO: 34), and CDR3β—CASSEFWLTQETQYF (SEQ ID NO: 35), or variants thereof each having up to three amino acid substitutions, additions or deletions; or (iv) CDR1α—DSVNN (SEQ ID NO: 39), CDR2α—IPSGT (SEQ ID NO: 40), CDR3α—CAVSHGRGGATNKLIF (SEQ ID NO: 41), CDR1β—MNHNS (SEQ ID NO: 44), CDR2β—SASEGT (SEQ ID NO: 45), and CDR3β—CASDRVLGYEQYF (SEQ ID NO: 46), or variants thereof each having up to three amino acid substitutions, additions or deletions. In some embodiments, the TCR comprises: (i) an α chain variable domain comprising the amino acid sequence of SEQ ID NO: 9 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 14 or a variant thereof having at least 70%, at least 75%, at least 80%, at least 85%, at least 90%, at least 95%, at least 98%, or at least 99% sequence identity thereto; (ii) an α chain variable domain comprising the amino acid sequence of SEQ ID NO: 20 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 25 or a variant thereof having at least 70%, at least 75%, at least 80%, at least 85%, at least 90%, at least 95%, at least 98%, or at least 99% sequence identity thereto; (iii) an α chain variable domain comprising the amino acid sequence of SEQ ID NO: 31 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 36 or a variant thereof having at least 70%, at least 75%, at least 80%, at least 85%, at least 90%, at least 95%, at least 98%, or at least 99% sequence identity thereto; or (iv) an α chain variable domain comprising the amino acid sequence of SEQ ID NO: 42 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 47 or a variant thereof having at least 70%, at least 75%, at least 80%, at least 85%, at least 90%, at least 95%, at least 98%, or at least 99% sequence identity thereto. In some embodiments, the TCR comprises: (i) an α chain comprising the amino acid sequence of SEQ ID NO: 10 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 15 or 16, or variants thereof having at least 70%, at least 75%, at least 80%, at least 85%, at least 90%, at least 95%, at least 98%, or at least 99% sequence identity thereto; (ii) an α chain comprising the amino acid sequence of SEQ ID NO: 21 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 26 or 27, or variants thereof having at least 70%, at least 75%, at least 80%, at least 85%, at least 90%, at least 95%, at least 98%, or at least 99% sequence identity thereto; (iii) an α chain comprising the amino acid sequence of SEQ ID NO: 32 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 37 or 38, or variants thereof having at least 70%, at least 75%, at least 80%, at least 85%, at least 90%, at least 95%, at least 98%, or at least 99% sequence identity thereto; or (iv) an α chain comprising the amino acid sequence of SEQ ID NO: 43 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 48 or 49, or variants thereof having at least 70%, at least 75%, at least 80%, at least 85%, at least 90%, at least 95%, at least 98%, or at least 99% sequence identity thereto. The TCR may be restricted to HLA-A*0301. The hTERT peptide may comprise or consist of the amino acid sequence of SVLNYERARR (SEQ ID NO: 5) or a variant thereof having up to three amino acid substitutions, additions or deletions

In one aspect, the TCR binds to a Survivin peptide when presented by an MHC. Suitably, the TCR comprises a CDR3α comprising the amino acid sequence of CALDRMDSSYKLIF (SEQ ID NO: 54) or a variant thereof having up to three amino acid substitutions, additions or deletions, and a CDR3β comprising the amino acid sequence of CASSYDQDGEAFF (SEQ ID NO: 59) or a variant thereof having up to three amino acid substitutions, additions or deletions. In some embodiments, the TCR comprises the following CDR sequences: CDR1α—NYSPAY (SEQ ID NO: 52), CDR2α—IRENEKE (SEQ ID NO: 53), CDR3α—CALDRMDSSYKLIF (SEQ ID NO: 54), CDR1β—MNHEY (SEQ ID NO: 57), CDR2β—SVGAGI (SEQ ID NO: 58), and CDR3β—CASSYDQDGEAFF (SEQ ID NO: 59), or variants thereof each having up to three amino acid substitutions, additions or deletions. In some embodiments, the TCR comprises an α chain variable domain comprising the amino acid sequence of SEQ ID NO: 55 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 60 or a variant thereof having at least 70%, at least 75%, at least 80%, at least 85%, at least 90%, at least 95%, at least 98%, or at least 99% sequence identity thereto. In some embodiments, the TCR comprises an α chain comprising the amino acid sequence of SEQ ID NO: 56 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 61 or 62, or variants thereof having at least 70%, at least 75%, at least 80%, at least 85%, at least 90%, at least 95%, at least 98%, or at least 99% sequence identity thereto. The TCR may be restricted to HLA-A*0201. The Survivin peptide may comprise or consist of the amino acid sequence of LMLGEFLKL (SEQ ID NO: 51) or a variant thereof having up to three amino acid substitutions, additions or deletions.

In some embodiments, the TCR comprises one or more mutations at the α chain/β chain interface, such that when the α chain and the β chain are expressed in a T-cell, the frequency of mispairing between said chains and endogenous TCR α and β chains is reduced. In some embodiments, the TCR comprises one or more mutations at the α chain/β chain interface, such that when the α chain and the β chain are expressed in a T-cell, the level of expression of the TCR α and β chains is increased. In some embodiments, the one or more mutations introduce a cysteine residue into the constant region domain of each of the α chain and the β chain, wherein the cysteine residues are capable of forming a disulphide bond between the α chain and the β chain. In some embodiments, the one or more mutations are at amino acid positions selected from those disclosed in Table 1 of Boulter, J. M et al. (2003) Protein Engineering 16:707-711.

In some embodiments, the TCR comprises one or more mutations to remove one or more N-glycosylation sites (see, for example, Kuball, J et al. (2009) J Exp Med 206:463-75). Suitably, the N-glycosylation sites are in the TCR constant domains. In some embodiments, the mutation is a substitution of the amino acid N in an N-X-S/T motif with the amino acid Q. For example, the substitution may occur at one or more of the positions: TCR alpha constant gene position 36, 90 or 109; and/or TCR beta constant gene position 85.6. In some embodiments, the substitution is at position 36 of the TCR alpha constant gene.

In some embodiments, the TCR comprises a murinised constant region.

In some embodiments, the TCR is a soluble TCR.

In another aspect, the invention provides a polynucleotide encoding the α chain of a T-cell receptor (TCR) according to the invention, and/or the β chain of a TCR according to the invention. In some embodiments, the polynucleotide encodes the α chain linked to the β chain. In some embodiments, the polynucleotide further encodes one or more short interfering RNA (siRNA) or other agents capable of reducing or preventing expression of one or more endogenous TCR genes.

In another aspect, the invention provides a vector comprising a polynucleotide according to the invention. In some embodiments, the vector is a plasmid or a viral vector. In some embodiments, the vector comprises a polynucleotide, which encodes one or more CD3 chains, CD8, a suicide gene and/or a selectable marker.

In another aspect, the invention provides a cell comprising a TCR of the invention, a polynucleotide of the invention or a vector of the invention. In some embodiments, the cell further comprises a vector which encodes one or more CD3 chains, CD8, a suicide gene and/or a selectable marker.

The cell of the invention may be any suitable cell. Suitably, the cell is a T-cell, a lymphocyte, or a stem cell. In some embodiment, the cell is selected from the group consisting of CD4+ cells, CD8+ cells, naive T-cells, memory stem T-cells, central memory T-cells, double negative T-cells, effector memory T-cells, effector T-cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, cytokine-induced killer (CIK) cells, hematopoietic stem cells and pluripotent stem cells. In some embodiments, the cell is a T-cell. In some embodiments, the cell is a T-cell which has been isolated from a subject.

In some embodiments, an endogenous gene encoding a TCR α chain and/or an endogenous gene encoding a TCR β chain in the cell is disrupted, suitably such that the endogenous gene encoding a TCR α chain and/or the endogenous gene encoding a TCR β chain is not expressed. In some embodiments, the endogenous gene encoding a TCR α chain and/or the endogenous gene encoding a TCR β chain is disrupted by insertion of an expression cassette comprising a polynucleotide sequence encoding the TCR of the invention. In some embodiments, one or more endogenous genes encoding an MHC is disrupted, suitably wherein the cell is a non-alloreactive universal T-cell. In some embodiments, an endogenous gene involved in persistence, expansion, activity, resistance to exhaustion/senescence/inhibitory signals, homing capacity, or other T-cell functions is disrupted, suitably wherein the endogenous gene involved in persistence, expansion, activity, resistance to exhaustion/senescence/inhibitory signals, homing capacity, or other T-cell functions is selected from the group consisting of PD1, TIM3, LAG3, 2B4, KLRG1, TGFbR, CD160, TIGIT, CTLA4 and CD39. In some embodiments, the endogenous gene involved in persistence, expansion, activity, resistance to exhaustion/senescence/inhibitory signals, homing capacity, or other T-cell functions is disrupted by integration of an expression cassette, wherein the expression cassette comprises a polynucleotide sequence encoding a TCR of the invention.

In another aspect, the invention provides a T-cell genetically engineered (e.g. genetically edited) to modify the persistence, expansion, activity, resistance to exhaustion/senescence/inhibitory signals, homing capacity or other T cell functions, wherein the T-cell expresses a TCR α chain of the invention and/or a TCR β chain of the invention.

In another aspect, the invention provides a T cell genetically engineered (e.g. genetically edited) by a protocol which comprises the step of targeted integration of an expression cassette into an endogenous gene involved in persistence, expansion, activity, resistance to exhaustion/senescence/inhibitory signals, homing capacity or other T-cell functions disrupted by an artificial nuclease, wherein the expression cassette comprises a polynucleotide sequence encoding TCR α chain of the invention and/or a TCR β chain of the invention.

In another aspect, the invention provides a method of preparing a cell, which comprises the step of introducing the polynucleotide or vector of the invention into a cell in vitro, ex vivo or in vivo, for example by transfection or transduction.

In another aspect, the invention provides a method of preparing a cell, which comprises the step of transducing a cell in vitro, ex vivo or in vivo with one or more vectors of the invention.

In some embodiments, the cell to be transduced or transfected is selected from the group consisting of T-cells, lymphocytes or stem cells, such as hematopoietic stem cells or induced pluripotent stem cells (iPS). In some embodiments, the cell to be transduced or transfected is selected from the group consisting of CD4+ cells, CD8+ cells, Th0 cells, TO cells, Th1 cells, Tc1 cells, Th2 cells, Tc2 cells, Th17 cells, Th22 cells, gamma/delta T-cells, natural killer (NK) cells, natural killer T (NKT) cells, double negative T-cells, naive T-cells, memory stem T-cells, central memory T-cells, effector memory T-cells, effector T cells, cytokine-induced killer (CIK) cells, hematopoeitic stem cells and pluripotent stem cells.

In some embodiments, the method of preparing a cell comprises the step of T-cell editing, which comprises disrupting an endogenous gene encoding a TCR α chain and/or an endogenous gene encoding a TCR β chain with an artificial nuclease, optionally wherein the artificial nuclease is selected from the group consisting of zinc finger nucleases (ZFNs), transcription activator-like effector nucleases (TALENs) and CRISPR/Cas systems.

In some embodiments, the method of preparing a cell comprises the step of targeted integration of an expression cassette into the endogenous gene encoding the TCR α chain and/or the endogenous gene encoding the TCR β chain disrupted by the artificial nuclease, wherein the expression cassette comprises a polynucleotide sequence encoding the TCR of the invention or a polynucleotide sequence of the invention.

In some embodiments, the method of preparing a cell comprises the step of disrupting one or more endogenous genes encoding an MHC, suitably wherein the cell prepared by the method is a non-alloreactive universal T-cell.

In some embodiments, the method of preparing a cell comprises the step of disrupting one or more endogenous genes to modify the persistence, expansion, activity, resistance to exhaustion/senescence/inhibitory signals, homing capacity, or other T-cell functions, optionally wherein the method comprises the step of targeted integration of an expression cassette into an endogenous gene involved in persistence, expansion, activity, resistance to exhaustion/senescence/inhibitory signals, homing capacity, or other T-cell functions disrupted by an artificial nuclease, wherein the expression cassette comprises a polynucleotide sequence encoding the TCR of the invention, suitably wherein the endogenous gene is selected from the group consisting of PD1, TIM3, LAG3, 2B4, KLRG1, TGFbR, CD160, TIGIT, CTLA4 and CD39.

In another aspect, the invention provides a chimeric molecule comprising the TCR of the invention, or a portion thereof, conjugated to a non-cellular substrate, a toxin and/or an antibody. In some embodiments, the non-cellular substrate is selected from the group consisting of nanoparticles, exosomes and other non-cellular substrates.

In another aspect, the invention provides a pharmaceutical composition comprising the TCR of the invention, the polynucleotide of the invention, the vector of the invention, the cell of the invention, a cell prepared by the method of the invention, or the chimeric molecule of the invention.

In another aspect, the invention provides the cell of the invention or a cell prepared by the method of the invention for use in adoptive cell transfer, suitably adoptive T-cell transfer, optionally wherein the adoptive T-cell transfer is allogenic adoptive T-cell transfer, autologous adoptive T-cell transfer, or universal non-alloreactive adoptive T-cell transfer.

In another aspect, the invention provides the TCR of the invention, the polynucleotide of the invention, the vector of the invention, the cell of the invention, a cell prepared by the method of the invention, the chimeric molecule of the invention, or the pharmaceutical composition of the invention for use in therapy.

In another aspect, the invention provides the TCR of the invention, the polynucleotide of the invention, the vector of the invention, the cell of the invention, a cell prepared by the method of the invention, a chimeric molecule of the invention, or a pharmaceutical composition of the invention for use in treating and/or preventing a proliferative disorder.

In another aspect, the invention provides a method for treating and/or preventing a proliferative disorder, which comprises the step of administering the TCR of the invention, the polynucleotide of the invention, the vector of the invention, the cell of the invention, a cell prepared by the method of the invention, a chimeric molecule of the invention, or a pharmaceutical composition of the invention to a subject in need thereof.

The proliferative disorder may be a hematological malignancy or a solid tumor. Suitably, the hematological malignancy is selected from the group consisting of acute myeloid leukemia (AML), chronic myeloid leukemia (CML), lymphoblastic leukemia, acute lymphocytic leukemia (ALL), myelodisplastic syndromes, lymphoma, multiple myeloma, non-Hodgkin lymphoma, and Hodgkin lymphoma. Suitably, the solid tumor is selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, oral cancer, hepatocellular carcinoma, bladder cancer, endometrial cancer, neuroblastoma, rabdomyosarcoma, liver cancer, melanoma, prostate cancer, renal cancer, soft tissue sarcoma, urothelial cancer, biliary cancer, glioblastoma, cervical cancer, mesothelioma and colorectal cancer. In preferred embodiments, the proliferative disorder is acute myeloid leukemia (AML).

### DESCRIPTION OF DRAWINGS

FIG. 1—Isolation of hTERT₆₆₃₋₆₇₂—specific and Surviving₆₉₋₁₀₄-specific T cells from AML patients' peripheral blood using Dextramer technology

The experimental procedure employing Dextramer technology was efficient in enriching for hTERT-specific and Survivin-specific T cells. (A) hTERT-specific T cells, from different cell cultures (#1, #2, #3) derived from the same patient, were isolated and expanded in vitro. The percentage of Dextramer⁺ events on total CD3⁺ T cells before and after sorting and in vitro expansion is reported. (B) Survivin-specific T cells were isolated as hTERT-specific T cells, without performing a prior in vitro expansion. The percentage of Dextramer⁺ events on total CD3⁺ T cells before sorting is reported.

FIG. 2—Sequencing of hTERT-specific and Survivin-specific T cells

CDR3-alpha (left) and -beta (right) sequences of hTERT-specific cell cultures. The first three dominant clones are reported in darker colors.

FIG. 3—Sequencing of sorted Survivin-specific T cells

CDR3-alpha (upper) and -beta (lower) sequences of the Survivin-specific T cell population. The dominant clone is reported in darker colors.

FIG. 4—hTERT₆₆₃₋₆₇₂-specific T cells recognize target cell lines

(A) hTERT-specific transgenic T cells were co-cultured with an HLA-A0301⁺ EBV cell line, loaded with the hTERT₆₆₃₋₆₇₂ peptide or an unrelated one as control, for 72 hours in a 10:1 and 1:1 effector to target ratio. Killing is reported as elimination index calculated according to the formula [1-(number of hTERT-peptide-pulsed alive cell lines cultured with edited T cells/number of unrelated-peptide-pulsed alive cell lines cultured with edited T cells)]. (B) hTERT-specific transgenic T cells were co-cultured with an HLA-A0301⁺ EBV cell line, loaded with the hTERT₆₆₃₋₆₇₂ peptide or an unrelated one as control, for 6 hours to assess cytokine production. Frequency of cells producing CD107a (left) and IFNγ (right) is reported. (C) hTERT-specific transgenic T cells were co-cultured with an HLA-A0301⁺ EBV cell line, loaded with the hTERT₆₆₃₋₆₇₂ peptide or an unrelated one as control, for 12 hours in a 1:1 effector to target ratio. Killing is represented as area covered (μm²) by cells undergoing apoptosis (caspase 3/7 activation) overtime. Data are represented as mean±SEM (A) and as mean±SD (B, C). **P<0.01 ****P<0.0001 by Student t-test (B) or two-way ANOVA (C).

FIG. 5—Survivin-specific T cells recognize target cell lines

(A) Survivin-specific transgenic T cells were co-cultured with T2 cell line, loaded with the specific Survivin peptide or an unrelated one as control, for 72 hours in a 10:1 and 1:1 effector to target ratio. Killing is reported as elimination index calculated according to the formula [1-(number of Survivin-pulsed alive cell lines cultured with edited T cells/number of unrelated-peptide-pulsed alive cell lines cultured with edited T cells)]. (B) Survivin-specific transgenic T cells were co-cultured with T2 cell line, loaded with the specific Survivin peptide or an unrelated one as control, for 6 hours to assess cytokine production. Frequency of cells producing CD107a (left) and TNFα (right) is reported. (C) Survivin-specific transgenic T cells were co-cultured with T2 cell line, loaded with the specific Survivin peptide or an unrelated one as control, for 12 hours in a 1:1 effector to target ratio. Killing is represented as area covered (μm²) by cells undergoing apoptosis (caspase 3/7 activation) overtime. Data are represented as mean±SD. ****P<0.0001 by two-way ANOVA (C).

FIG. 6—hTERT₆₆₃₋₆₇₂-specific T cells recognize primary AML blasts

(A) hTERT-specific transgenic T cells were co-cultured with hTERT⁺ primary AML blasts expressing (target blasts) or not (control blasts) the HLA-A0301⁺ allele in 1:1 effector to target ratio. After 24 hours, elimination index was calculated as [1-(number of alive blasts cultured with edited T cells/number of alive blasts alone)]. (B,C) hTERT-specific transgenic T cells were co-cultured hTERT⁺ primary AML blasts expressing (target blasts, B) or not (control blasts, C) the HLA-A0301⁺ allele in 1:1 effector to target ratio. Killing is represented as area covered (μm²) by cells undergoing apoptosis (caspase 3/7 activation) overtime. Taken together, hTERT #1, hTERT #2 and hTERT #4 had the most potent anti-leukemic activity at high specificity, mediating the specific lysis of AML blasts harboring the correct HLA. Data are presented as means±SD. *P<0.05, **<0.005, ***P<0.0005, ****P<0.0001 by Student t-test (A) or two-way ANOVA (B).

FIG. 7—Surivivn₉₆₋₁₀₄-specific T cells recognize primary AML blasts

(A) Survivin-specific transgenic T cells were co-cultured with Survivin⁺ primary AML blasts expressing (target blasts) or not (control blasts) the HLA-A0201⁺ allele in 1:1 effector to target ratio. After 24 hours, elimination index was calculated as [1-(number of alive blasts cultured with edited T cells/number of alive blasts alone)]. (B) Survivin-specific transgenic T cells were co-cultured with Survivin⁺ primary AML blasts expressing (target blasts, upper) or not (control blasts, lower) the HLA-A0201⁺ allele in 1:1 effector to target ratio. Killing is represented as area covered (μm²) by cells undergoing apoptosis (caspase 3/7 activation) overtime. Taken together, these data demonstrate HLA-specific AML lysis. Data are presented as means±SD. ****P<0.0001, by Student t-test (A) or two-way ANOVA (B).

FIG. 8—hTERT-TCR T cells delay tumor outgrowth in tumor-bearing NSG mice

(A) Timeline of the AML in vivo model. hTERT #1 TCR-engineered T cells were used as effector cells. (B) T-cell activation (measured through the expression of the HLA-DR marker) at different time points after lymphocyte infusion and (C) leukemic blast count at the endpoint of the experiment in mice either treated with engineered hTERT #1 TCR T cells or left untreated (AML only). Data are meant±sem. *p<0.05 by Mann-Whitney test. TCR, T-cell receptor; AML, primary acute myeloid leukemia; HLA, human leukocyte antigen; hTERT, human telomerase reverse transcriptase.

### DETAILED DESCRIPTION

Various preferred features and embodiments of the present invention will now be described by way of non-limiting examples. This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. Unless otherwise indicated, any nucleic acid sequences are written left to right in 5′ to 3′ orientation; amino acid sequences are written left to right in amino to carboxy orientation, respectively.

The publications discussed herein are provided solely for their disclosure prior to the filing date of the present application. Nothing herein is to be construed as an admission that such publications constitute prior art to the claims appended hereto. All publications mentioned in the specification are herein incorporated by reference.

### T-Cell Receptors (TCRs)

In one aspect, the present invention provides a T-cell receptor (TCR) which binds to a Human Telomerase Reverse Transcriptase (hTERT) peptide or a Survivin peptide when presented by a major histocompatibility complex (MHC). The present invention also provides an α chain or a β chain from such a T cell receptor.

As used herein the term “T-cell receptor” (TCR) may refer to molecule capable of recognising a peptide when presented by an MHC molecule. TCRs may be found on the surface of T-cells and are responsible for recognising an antigen peptide bound to an MHC molecule. During antigen processing, antigens are degraded inside cells and then antigen peptides carried to the cell surface by MHC molecules. Binding of the TCR to the peptide:MHC complex results in activation of the T lymphocyte on which the TCR is expressed through a series of biochemical events mediated by associated enzymes, co-receptors, and specialized accessory molecules.

TCR affinity for the peptide may be determined by the association (kₒₙ) and dissociation rates (k_{off}) and represented as the equilibrium dissociation constant (KD). The TCR affinity may be determined by any suitable method, for example by titration calorimetry, surface plasmon resonance (SPR) or fluorescence microscopy-based methods (see e.g. Piepenbrink, K. H., et al., 2009. Methods Enzymol, 466, pp: 359-381). The TCR may bind to a peptide when presented by an MHC with an affinity of about 100 μM or less, about 50 μM or less, about 40 μM or less, about 30 μM or less, about 20 μM or less, or about 10 μM or less. The TCR may bind to a peptide when presented by an MHC with an affinity of about 0.1 μM to about 100 μM, about 1 μM to about 100 μM, or about 5 μM to about 100 μM. The TCR may bind to a peptide when presented by an MHC with an affinity of about 0.1 μM to about 10 μM, about 1 μM to about 10 μM, or about 5 μM to about 10 μM.

The naturally-occurring TCR heterodimer consists of an alpha (α) and beta (β) chain in around 95% of T-cells, whereas around 5% of T-cells have TCRs consisting of gamma (γ) and delta (δ) chains. Each chain of a natural TCR is a member of the immunoglobulin superfamily and possesses an N-terminal immunoglobulin (lg)-variable (V) region, a lg-constant (C) region, a transmembrane/cell membrane-spanning region, and a short cytoplasmic tail at the C-terminal end. The TCR of the present invention may be a heterodimer of two chains α and β (or optionally γ and δ) or it may be a single chain TCR construct.

A variable domain may determine the specificity of the TCR. The variable domain of both the TCR α chain and β chain have three hypervariable or complementarity determining regions (CDRs). A TCR α chain or β chain, for example, comprises a CDR1, a CDR2, and a CDR3 in amino to carboxy terminal order. In general, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. Suitable variable domains are described herein.

A TCR of the invention may comprise a constant domain. A constant domain may allow the TCR to associate with other molecules like CD3 which possess three distinct chains (γ, δ, and ε) in mammals and the ζ-chain. These accessory molecules have negatively charged transmembrane regions and are vital to propagating the signal from the TCR into the cell. The CD3- and ζ-chains, together with the TCR, form what is known as the T cell receptor complex.

The constant domain may consist of short connecting sequences in which a cysteine residue forms a disulfide bond, making a link between the two chains. The constant domain may comprise an Ig-constant (C) region, a transmembrane/cell membrane-spanning region and a cytoplasmic tail.

An α chain of a TCR of the present invention may comprise a constant domain encoded by a TRAC gene. An α chain constant domain may have the amino acid sequence set out in UniProt entry P01848. An example α chain constant domain has the amino acid sequence set out below in SEQ ID NO: 1.

A TCR of the invention may comprise an α chain constant domain comprising or consisting of the amino acid sequence of SEQ ID NO: 1 or a variant thereof having at least 70%, at least 75%, at least 80%, at least 85%, at least 90%, at least 95%, at least 96%, at least 97%, at least 98%, or at least 99% sequence identity thereto.

Example α chain constant domain
(SEQ ID NO: 1)
IQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKS
FETDTNLNFQNLSVIGFRILLLKVAGENLLMTLRLWSS

A β chain of a TCR of the present invention may comprise a constant domain encoded by a TRBC1 or a TRBC2 gene. A β chain constant domain may have the amino acid sequence set out in UniProt entry P01850 or A0A5B9. An example β chain constant domain encoded by a TRBC1 gene has the amino acid sequence set out below in SEQ ID NO: 2. An example β chain constant domain encoded by a TRBC2 gene has the amino acid sequence set out below in SEQ ID NO: 3.

A TCR of the invention may comprise a β chain constant domain comprising the amino acid sequence of SEQ ID NO: 2 or 3 or a variant thereof having at least 70%, at least 75%, at least 80%, at least 85%, at least 90%, at least 95%, at least 96%, at least 97%, at least 98%, or at least 99% sequence identity thereto.

Example ß chain constant domain
(SEQ ID NO: 2)
DLNKVFPPEVAVFEPSEAEISHTQKATLVCLATGFFPDHVELSWWVNGKEV
HSGVSTDPQPLKEQPALNDSRYCLSSRLRVSATFWQNPRNHFRCQVQFYGL
SENDEWTQDRAKPVTQIVSAEAWGRADCGFTSVSYQQGVLSATILYEILLG
KATLYAVLVSALVLMAMVKRKDF
Example ß chain constant domain
(SEQ ID NO: 3)
DLKNVFPPEVAVFEPSEAEISHTQKATLVCLATGFYPDHVELSWWVNGKEV
HSGVSTDPQPLKEQPALNDSRYCLSSRLRVSATFWONPRNHFRCQVQFYGL
SENDEWTQDRAKPVTQIVSAEAWGRADCGFTSESYQQGVLSATILYEILLG
KATLYAVLVSALVLMAMVKRKDSRG

The TCR of the invention may have one or more additional cysteine residues in each of the α and β chains such that the TCR may comprise two or more disulphide bonds in the constant domains.

The signal from the T cell complex may be enhanced by simultaneous binding of the MHC molecules by a specific co-receptor. For helper T-cells, this co-receptor may be CD4 (specific for class II MHC); whereas for cytotoxic T-cells, this co-receptor may be CD8 (specific for class I MHC). The co-receptor can allow prolonged engagement between the antigen presenting cell and the T cell and can recruit essential molecules (e.g., LCK) inside the cell involved in the signalling of the activated T lymphocyte.

The TCR of the invention may be a hybrid TCR comprising sequences derived from more than one species. For example, it has been found that murine TCRs are more efficiently expressed in human T-cells than human TCRs. The TCR may therefore comprise a human variable domain and murine sequences within a constant domain. A disadvantage of this approach is that the murine constant sequences may trigger an immune response, leading to rejection of the transferred T-cells. However, the conditioning regimens used to prepare patients for adoptive T-cell therapy may result in sufficient immunosuppression to allow the engraftment of T-cells expressing murine sequences.

A TCR of the invention may be a soluble TCR, e.g. omitting or altering one or more constant domains. Other suitable methods for engineering soluble TCRs are known in the art (see e.g. Robinson, R. A., et al., 2021. The FEBS Journal, 288 (21), pp. 6159-6173).

In some embodiments, the TCR comprises one or more mutations to remove one or more N-glycosylation sites. Suitably, the N-glycosylation sites are in the TCR constant domains. Deletion of N-glycosylation sites in TCR constant domains is described in Kuball, J et al. (2009) J Exp Med 206:463-75. In some embodiments, the one or more mutations are substitutions of the amino acid N in an N-X-S/T motif with the amino acid Q. For example, the substitution may at one or more of the positions: TCR alpha constant gene position 36, 90 or 109; and/or TCR beta constant gene position 85.6. Suitably, the substitution is at position 36 of the TCR alpha constant gene. Mutations of TCR constant domains disclosed herein may be described based on a numbering convention in which the first amino acid of each of SEQ ID NOs: 1-3 is assigned to be position 2.

### Complementarity Determining Regions (CDRs)

T-cell receptor diversity is focused on CDR3 and this region is primarily responsible for antigen recognition. The sequences of the CDR3 regions of the TCR of the invention may be selected from those described herein. A TCR of the present invention may comprise CDRs that comprise or consist of a CDR3α and a CDR3β pair described herein.

The portion of the TCR that establishes the majority of the contacts with the antigenic peptide bound to the major histocompatibility complex (MHC) is the complementarity determining region 3 (CDR3), which is unique for each T cell clone. The CDR3 region is generated upon somatic rearrangement events occurring in the thymus and involving non-contiguous genes belonging to the variable (V), diversity (D, for β and δ chains) and joining (J) genes. Furthermore, random nucleotides inserted/deleted at the rearranging loci of each TCR chain gene greatly increase diversity of the highly variable CDR3 sequence. Thus, the frequency of a specific CDR3 sequence in a biological sample indicates the abundance of a specific T cell population. The great diversity of the TCR repertoire in healthy human beings provides a wide range protection towards a variety of foreign antigens presented by MHC molecules on the surface of antigen presenting cells. In this regard, it is of note that theoretically up to 1015 different TCRs can be generated in the thymus.

The sequences of the CDR1 and CDR2 regions of the TCR of the invention may also be selected from those described herein. A TCR of the present invention may comprise CDRs that comprise of consist of CDR1α, CDR2α, CDR3α, CDR1β, CDR2β, and CDR3β sets described herein.

The CDRs may, for example, comprise one, two, or three substitutions, additions or deletions from the given sequence, provided that the TCR retains the capacity to bind the corresponding immunogenic peptide when presented by an MHC molecule. In some embodiments, each CDR3 comprises up to three substitutions, additions or deletions, up to two substitutions, additions or deletions, up to one substitution, addition or deletion, or any combination thereof. In some embodiments, each CDR3 comprises up to three substitutions, up to two substitutions, up to one substitution, or any combination thereof. In some embodiments, each CDR comprises up to three substitutions, additions or deletions, up to two substitutions, additions or deletions, up to one substitution, addition or deletion, or any combination thereof. In some embodiments, each CDR comprises up to three substitutions, up to two substitutions, up to one substitution, or any combination thereof.

### Major Histocompatability Complex (MHC) Molecules

Typically, TCRs bind to peptides as part of peptide:MHC complex. The TCR of the present invention may bind to an MHC I and/or MHC II peptide complex. The MHC peptide complex may be on the surface of an antigen presenting cell, such as a dendritic cell or a B cell, or any other cell, including cancer cells, or it may be immobilised by, for example, coating on to a bead or plate.

The human leukocyte antigen system (HLA) is the name of the gene complex which encodes MHC in humans and includes HLA class I antigens (A, B & C) and HLA class II antigens (DP, DQ, & DR). HLA alleles A, B and C present peptides derived mainly from intracellular proteins, e.g. proteins expressed within the cell. The TCR is restricted to a human leukocyte antigen (HLA) allele.

During T-cell development in vivo, T-cells undergo a positive selection step to ensure recognition of self MHCs followed by a negative step to remove T-cells that bind too strongly to MHC which present self-antigens. As a consequence, certain T-cells and the TCRs they express will only recognise peptides presented by certain types of MHC molecules, i.e. those encoded by particular HLA alleles. This is known as HLA restriction. The preferred MHC binding motifs of different HLA alleles are disclosed in The Immune Epitope Database (IEDB) (Vita, R., et al., 2019. Nucleic acids research, 47(D1), pp. D339-D343).

The TCR of the present invention may be restricted to a HLA-A, HLA-B or a HLA-C allele. Suitably, the TCR of the present invention is restricted to a HLA-A allele. Suitably, the TCR of the present invention may be restricted to a HLA-A*03 or a HLA-A*02 allele. Suitably, the TCR of the present invention is restricted to HLA-A*0301 or HLA-A*0201.

### Immunogenic Peptides

The TCRs of the present invention bind to an immunogenic peptide when presented by an MHC.

As used herein, the term “immunogenic peptide” may refer to a peptide which is capable of being presented by an MHC molecule and subsequently recognised by a TCR. The immunogenic peptide may be derived from e.g. an AML-associated antigen. As used herein, the term “peptide” may refer to a plurality of amino acid residues linked by peptide bonds. A peptide may consist of less than about 30, less than about 25, less than about 20, less than 19, less than 18, less than 17, less than 16, less than 15, less than 14, less than 13, less than 12, less than 11, less than 10, less than 9, less than 8, less than 7, less than 6, or less than 5 amino acid residues in length. Suitably, a peptide is about 5 to 20 amino acids in length or about 8 to 15 amino acid residues in length.

As used herein, the term “protein” may include single-chain polypeptide molecules as well as multiple-polypeptide complexes where individual constituent polypeptides are linked by covalent or non-covalent means. As used herein, the term “polypeptide” may refer to a polymer in which the monomers are amino acids and are joined together through peptide or disulphide bonds.

As used herein, an “AML-associated antigen” may refer to an antigenic protein produced in acute myeloid leukaemia cells. AML-associated antigens may act as useful tumour markers and as potential candidates for use in immunotherapy. The present inventors have identified Human Telomerase Reverse Transcriptase (hTERT) and Survivin as potential AML-associated antigens.

Human Telomerase Reverse Transcriptase (hTERT) Specific TCRs

In one aspect, the present invention provides a TCR which binds to a Human Telomerase Reverse Transcriptase (hTERT) peptide when presented by an MHC.

Human Telomerase Reverse Transcriptase (hTERT)

Human Telomerase Reverse Transcriptase (hTERT) is a protein that is encoded by the TERT gene and is also known as Telomerase-associated protein 2 (TP2) or Telomerase Catalytic Subunit.

Telomerase is a ribonucleoprotein enzyme essential for the replication of chromosome termini in most eukaryotes that is active in progenitor and cancer cells. hTERT is the catalytic component of the teleromerase holoenzyme complex amd may catalyse the RNA-dependent extension of 3′-chromosomal termini with the 6-nucleotide telomeric repeat unit, 5′-TTAGGG-3′ (see e.g. Poole, J. C., et al., 2001. Gene, 269 (1-2), pp. 1-12)

The hTERT protein may have the amino acid sequence set out in UniProt entry 014746. An example hTERT protein has the amino acid sequence set out below in SEQ ID NO: 4.

Example hTERT protein
(SEQ ID NO: 4)
MPRAPRCRAVRSLLRSHYREVLPLATFVRRLGPQGWRLVQRGDPAAFRALV
AQCLVCVPWDARPPPAAPSFRQVSCLKELVARVLQRLCERGAKNVLAFGFA
LLDGARGGPPEAFTTSVRSYLPNTVTDALRGSGAWGLLLRRVGDDVLVHLL
ARCALFVLVAPSCAYQVCGPPLYQLGAATQARPPPHASGPRRRLGCERAWN
HSVREAGVPLGLPAPGARRRGGSASRSLPLPKRPRRGAAPEPERTPVGQGS
WAHPGRTRGPSDRGFCVVSPARPAEEATSLEGALSGTRHSHPSVGRQHHAG
PPSTSRPPRPWDTPCPPVYAETKHFLYSSGDKEQLRPSFLLSSLRPSLTGA
RRLVETIFLGSRPWMPGTPRRLPRLPQRYWQMRPLFLELLGNHAQCPYGVL
LKTHCPLRAAVTPAAGVCAREKPQGSVAAPEEEDTDPRRLVQLLRQHSSPW
QVYGFVRACLRRLVPPGLWGSRHNERRFLRNTKKFISLGKHAKLSLQELTW
KMSVRDCAWLRRSPGVGCVPAAEHRLREEILAKFLHWLMSVYVVELLRSFF
YVTETTFQKNRLFFYRKSVWSKLQSIGIRQHLKRVQLRELSEAEVRQHREA
RPALLTSRLRFIPKPDGLRPIVNMDYVVGARTFRREKRAERLTSRVKALFS
VLNYERARRPGLLGASVLGLDDIHRAWRTFVLRVRAQDPPPELYFVKVDVT
GAYDTIPQDRLTEVIASIIKPQNTYCVRRYAVVQKAAHGHVRKAFKSHVST
LTDLQPYMRQFVAHLQETSPLRDAVVIEQSSSLNEASSGLEDVFLRFMCHH
AVRIRGKSYVQCQGIPQGSILSTLLCSLCYGDMENKLFAGIRRDGLLLRLV
DDFLLVTPHLTHAKTFLRTLVRGVPEYGCVVNLRKTVVNFPVEDEALGGTA
FVQMPAHGLFPWCGLLLDTRTLEVQSDYSSYARTSIRASLTENRGFKAGRN
MRRKLFGVLRLKCHSLFLDLQVNSLQTVCTNIYKILLLQAYRFHACVLQLP
FHQQVWKNPTFFLRVISDTASLCYSILKAKNAGMSLGAKGAAGPLPSEAVQ
WLCHQAFLLKLTRHRVTYVPLLGSLRTAQTQLSRKLPGTTLTALEAAANPA
LPSDFKTILD

hTERT Peptides

In one aspect, the invention provides a hTERT peptide. Suitably, the hTERT peptide is an isolated peptide.

As used herein, the term “hTERT peptide” may refer to a peptide comprising an amino acid sequence derived from a hTERT protein. Suitably, a hTERT peptide may comprise at least 5, at least 6, at least 7, at least 8, at least 9, at least 10, at least 11, at least 12, at least 13, at least 14, at least 15, at least 16, at least 17, at least 18, at least 19, at least 20, or at least 25 contiguous amino acid residues of a hTERT protein amino acid sequence.

The hTERT peptide may be an immunogenic peptide. The hTERT peptide may bind to any suitable MHC. Suitably, the hTERT peptide binds to an MHC encoded by a HLA-A, HLA-B or a HLA-C allele. Suitably, the hTERT peptide binds to an MHC encoded by a HLA-A allele. Suitably, Suitably, the hTERT peptide binds to an MHC encoded by a HLA-A*03 allele. Suitably, the hTERT peptide binds to an MHC encoded by HLA-A*0301.

An example hTERT peptide has the amino acid sequence set out below in SEQ ID NO: 5.

SVLNYERARR
(SEQ ID NO: 5)
Example hTERT peptide

The hTERT peptide may comprise or consist of the amino acid sequence of SVLNYERARR (SEQ ID NO: 5) or a variant thereof having up to three amino acid substitutions, additions or deletions. In some embodiments, the hTERT peptide comprises or consists of the amino acid sequence of SVLNYERARR (SEQ ID NO: 5) or a variant thereof having up to two amino acid substitutions, additions or deletions. In some embodiments, the hTERT peptide comprises or consists of the amino acid sequence of SVLNYERARR (SEQ ID NO: 5) or a variant thereof having up to one amino acid substitution, addition or deletion. In some embodiments, the hTERT peptide comprises or consists of the amino acid sequence of SVLNYERARR (SEQ ID NO: 5) or a variant thereof having up to three amino acid substitutions. In some embodiments, the hTERT peptide comprises or consists of the amino acid sequence of SVLNYERARR (SEQ ID NO: 5) or a variant thereof having up to two amino acid substitutions. In some embodiments, the hTERT peptide comprises or consists of the amino acid sequence of SVLNYERARR (SEQ ID NO: 5) or a variant thereof having up to one amino acid substitution. In some embodiments, the hTERT peptide comprises or consists of the amino acid sequence of SVLNYERARR (SEQ ID NO: 5).

Various uses of the hTERT peptide described herein are contemplated by the invention.

For example, the hTERT peptide described herein may be administered to a subject, e.g. a human subject. Administration of the hTERT peptide of the invention may elicit an immune response against cells expressing or overexpressing hTERT protein, i.e. the hTERT peptide may be an immunogenic hTERT peptide.

The hTERT peptide described herein may be used to screen for and/or identify new TCR sequences which bind to hTERT cells. For example, HLA-A0301⁺ target cells may be pulsed with a hTERT peptide mentioned in the invention and incubated with a T-cell population isolated from a donor. In this approach, expression of cytokines, e.g. CD107a and IFNγ, may be indicative of T-cells which recognise hTERT peptides.

hTERT-Specific TCR Sequences

The present inventors have determined the amino acid sequences of a hTERT-specific TCR, including the CDR regions, which are responsible for binding specificity for the hTERT peptide.

The hTERT-specific TCR may be restricted to any suitable MHC (e.g. one or more HLA allele). Suitably, the hTERT-specific TCR is restricted to a HLA-A, HLA-B or a HLA-C allele. Suitably, the hTERT-specific TCR is restricted to a HLA-A allele. Suitably, the hTERT-specific TCR is restricted to a HLA-A*03 allele. Suitably, the hTERT-specific TCR is restricted to HLA-A*0301.

Example hTERT-specific TCR amino acid sequences are provided in the tables below.

hTERT #1

| Chain | Region | Example amino acid sequence |
|---|---|---|
| alpha | CDR1α | DSASNY (SEQ ID NO: 6) |
| | CDR2α | IRSNVGE (SEQ ID NO: 7) |
| | CDR3α | CADWVDMRF (SEQ ID NO: 8) |
| | α | MTSIRAVFIFLWLQLDLVNGENVEQHPSTLSVQEGDSAVIKCTYSDSASN |
| | variable | YFPWYKQELGKRPQLIIDIRSNVGEKKDQRIAVTLNKTAKHFSLHITETQ |
| | domain | PEDSAVYFCADWVDMRFGAGTRLTVKPN (SEQ ID NO: 9) |
| | α chain | MTSIRAVFIFLWLQLDLVNGENVEQHPSTLSVQEGDSAVIKCTYSDSASN |
| | with | YFPWYKQELGKRPQLIIDIRSNVGEKKDQRIAVTLNKTAKHESLHITETQ |
| | TRAC | PEDSAVYFCADWVDMRFGAGTRLTVKPNIQNPDPAVYQLRDSKSSDKSVC |
| | constant | LFTDFDSQTNVSQSKDSDVYITDKTVLDMRSMDFKSNSAVAWSNKSDEAC |
| | domain | ANAFNNSIIPEDTFFPSPESSCDVKLVEKSFETDINLNFQNLSVIGFRIL |
| | | LLKVAGENLLMTLRLWSS (SEQ ID NO: 10) |
| beta | CDR1β | DFQATT (SEQ ID NO: 11) |
| | CDR2β | SNEGSKA (SEQ ID NO: 12) |
| | CDR3β | CSAPLDRGSNQPQHF (SEQ ID NO: 13) |
| | β | MLLLLLLLGPGISLLLPGSLAGSGLGAVVSQHPSWVICKSGTSVKIECRS |
| | variable | LDFQATTMFWYRQFPKQSLMLMATSNEGSKATYEQGVEKDKELINHASLT |
| | domain | LSTLTVTSAHPEDSSFYICSAPLDRGSNQPQHFGDGTRLSILE (SEQ |
| | | ID NO: 14) |
| | β chain | MLLLLLLLGPGISLLLPGSLAGSGLGAVVSQHPSWVICKSGTSVKIECRS |
| | with | LDFQATTMFWYRQFPKQSLMLMATSNEGSKATYEQGVEKDKELINHASLT |
| | TRBC1 | LSTLTVTSAHPEDSSFYICSAPLDRGSNQPQHFGDGTRLSILEDLNKVFP |
| | constant | PEVAVFEPSEAEISHTQKATLVCLATGFFPDHVELSWWVNGKEVHSGVST |
| | domain | DPQPLKEQPALNDSRYCLSSRLRVSATFWQNPRNHFRCQVQFYGLSENDE |
| | | WTQDRAKPVTQIVSAEAWGRADCGFTSVSYQQGVLSATILYEILLGKATL |
| | | YAVLVSALVLMAMVKRKDF (SEQ ID NO: 15) |
| | β chain | MLLLLLLLGPGISLLLPGSLAGSGLGAVVSQHPSWVICKSGTSVKIECRS |
| | with | LDFQATTMFWYRQFPKQSLMLMATSNEGSKATYEQGVEKDKFLINHASLT |
| | TRBC2 | LSTLTVTSAHPEDSSFYICSAPLDRGSNQPQHFGDGIRLSILEDLKNVEP |
| | constant | PEVAVFEPSEAEISHTQKATLVCLATGFYPDHVELSWWVNGKEVHSGVST |
| | domain | DPQPLKEQPALNDSRYCLSSRLRVSATFWQNPRNHFRCQVQFYGLSENDE |
| | | WTQDRAKPVTQIVSAEAWGRADCGFTSESYQQGVLSATILYEILLGKATL |
| | | YAVLVSALVLMAMVKRKDSRG (SEQ ID NO: 16) |

hTERT #2

| Chain | Region | Example amino acid sequence |
|---|---|---|
| alpha | CDR1α | TSGENG (SEQ ID NO: 17) |
| | CDR2α | NVLDGL (SEQ ID NO: 18) |
| | CDR3α | CAVSRPNSGYSTLTF (SEQ ID NO: 19) |
| | α | MWGVFLLYVSMKMGGTTGQNIDQPTEMTATEGAIVQINCTYQTSGFNGLE |
| | variable | WYQQHAGEAPTFLSYNVLDGLEEKGRESSFLSRSKGYSYLLLKELQMKDS |
| | domain | ASYLCAVSRPNSGYSTLTFGKGTMLLVSPD (SEQ ID NO: 20) |
| | α chain | MWGVFLLYVSMKMGGTTGQNIDQPTEMTATEGAIVQINCTYQTSGENGLE |
| | with | WYQQHAGEAPTFLSYNVLDGLEEKGRESSFLSRSKGYSYLLLKELQMKDS |
| | TRAC | ASYLCAVSRPNSGYSTLTFGKGTMLLVSPDIQNPDPAVYQLRDSKSSDKS |
| | constant | VCLFTDFDSQTNVSQSKDSDVYITDKTVLDMRSMDFKSNSAVAWSNKSDE |
| | domain | ACANAENNSIIPEDTFFPSPESSCDVKLVEKSFETDTNLNFQNLSVIGER |
| | | ILLLKVAGENLLMTLRLWSS (SEQ ID NO: 21) |
| beta | CDR1B | PRHDT (SEQ ID NO: 22) |
| | CDR2β | FYEKMQ (SEQ ID NO: 23) |
| | CDR3β | CASSVRTPSGQETQYF (SEQ ID NO: 24) |
| | β | MLSPDLPDSAWNTRLLCHVMLCLLGAVSVAAGVIQSPRHLIKEKRETATL |
| | variable | KCYPIPRHDTVYWYQQGPGQDPQFLISFYEKMQSDKGSIPDRESAQQFSD |
| | domain | YHSELNMSSLELGDSALYFCASSVRTPSGQETQYFGPGTRLLVLE (SEQ |
| | | ID NO: 25) |
| | β chain | MLSPDLPDSAWNTRLLCHVMLCLLGAVSVAAGVIQSPRHLIKEKRETATL |
| | with | KCYPIPRHDTVYWYQQGPGQDPQFLISFYEKMQSDKGSIPDRFSAQQFSD |
| | TRBC1 | YHSELNMSSLELGDSALYFCASSVRTPSGQETQYFGPGTRLLVLDLNKVF |
| | constant | PPEVAVFEPSEAEISHTQKATLVCLATGFFPDHVELSWWVNGKEVHSGVS |
| | domain | TDPQPLKEQPALNDSRYCLSSRLRVSATFWQNPRNHFRCQVQFYGLSEND |
| | | EWTQDRAKPVTQIVSAEAWGRADCGFTSVSYQQGVLSATILYEILLGKAT |
| | | LYAVLVSALVLMAMVKRKDF (SEQ ID NO: 26) |
| | β chain | MLSPDLPDSAWNTRLLCHVMLCLLGAVSVAAGVIQSPRHLIKEKRETATL |
| | with | KCYPIPRHDTVYWYQQGPGODPQFLISFYEKMQSDKGSIPDRFSAQQFSD |
| | TRBC2 | YHSELNMSSLELGDSALYFCASSVRTPSGQETQYFGPGTRLLVLEDLKNV |
| | constant | FPPEVAVFEPSEAEISHTQKATLVCLATGFYPDHVELSWWVNGKEVHSGV |
| | domain | STDPQPLKEQPALNDSRYCLSSRLRVSATFWQNPRNHERCQVQFYGLSEN |
| | | DEWTQDRAKPVTQIVSAEAWGRADCGFTSESYQQGVLSATILYEILLGKA |
| | | TLYAVLVSALVLMAMVKRKDSRG (SEQ ID NO: 27) |

hTERT #3

| Chain | Region | Example amino acid sequence |
|---|---|---|
| alpha | CDR1α | VSNAYN (SEQ ID NO: 28) |
| | CDR2α | GSKP (SEQ ID NO: 29) |
| | CDR3α | CAVETGGGATNKLIF (SEQ ID NO: 30) |
| | α | MALQSTLGAVWLGLLLNSLWKVAESKDQVFQPSTVASSEGAVVEIFCNHS |
| | variable | VSNAYNFFWYLHFPGCAPRLLVKGSKPSQQGRYNMTYERFSSSLLILQVR |
| | domain | EADAAVYYCAVETGGGATNKLIFGTGTLLAVQPN (SEQ ID NO: 31) |
| | α chain | MALQSTLGAVWLGLLLNSLWKVAESKDQVFQPSTVASSEGAVVEIFCNHS |
| | with | VSNAYNFFWYLHFPGCAPRLLVKGSKPSQQGRYNMTYERESSSLLILQVR |
| | TRAC | EADAAVYYCAVETGGGATNKLIFGTGTLLAVQPNIQNPDPAVYQLRDSKS |
| | constant | SDKSVCLFTDFDSQTNVSQSKDSDVYITDKTVLDMRSMDFKSNSAVAWSN |
| | domain | KSDFACANAENNSIIPEDTFFPSPESSCDVKLVEKSFETDINLNFQNLSV |
| | | IGFRILLLKVAGENLLMTLRLWSS (SEQ ID NO: 32) |
| beta | CDR1β | MNHNS (SEQ ID NO: 33) |
| | CDR2β | SASEGT (SEQ ID NO: 34) |
| | CDR3β | CASSEFWLTQETQYF (SEQ ID NO: 35) |
| | β | MSIGLLCCVAFSLLWASPVNAGVTQTPKFQVLKTGQSMTLOCAQDMNHNS |
| | variable | MYWYRQDPGMGLRLIYYSASEGTTDKGEVPNGYNVSRLNKREFSLRLESA |
| | domain | APSQTSVYFCASSEFWLTQETQYFGPGTRLLVLE (SEQ ID NO: 36) |
| | β chain | MSIGLLCCVAFSLLWASPVNAGVTQTPKFQVLKTGQSMTLQCAQDMNHNS |
| | with | MYWYRQDPGMGLRLIYYSASEGTTDKGEVPNGYNVSRLNKREFSLRLESA |
| | TRBC1 | APSQTSVYFCASSEFWLTQETQYFGPGTRLLVLEDLNKVEPPEVAVFEPS |
| | constant | EAEISHTQKATLVCLATGFFPDHVELSWWVNGKEVHSGVSTDPQPLKEQP |
| | domain | ALNDSRYCLSSRLRVSATFWQNPRNHERCQVQFYGLSENDEWTQDRAKPV |
| | | TQIVSAEAWGRADCGFTSVSYQQGVLSATILYEILLGKATLYAVLVSALV |
| | | LMAMVKRKDF (SEQ ID NO: 37) |
| | β chain | MSIGLLCCVAFSLLWASPVNAGVTQTPKFQVLKTGQSMTLQCAQDMNHNS |
| | with | MYWYRQDPGMGLRLIYYSASEGTTDKGEVPNGYNVSRLNKREFSLRLESA |
| | TRBC2 | APSQTSVYFCASSEFWLTQETQYFGPGTRLLVLEDLKNVEPPEVAVFEPS |
| | constant | EAEISHTQKATLVCLATGFYPDHVELSWWVNGKEVHSGVSTDPQPLKEQP |
| | domain | ALNDSRYCLSSRLRVSATFWQNPRNHERCQVQFYGLSENDEWTQDRAKPV |
| | | TQIVSAEAWGRADCGFTSESYQQGVLSATILYEILLGKATLYAVLVSALV |
| | | LMAMVKRKDSRG (SEQ ID NO: 38) |

hTERT #4

| Chain | Region | Example amino acid sequence |
|---|---|---|
| alpha | CDR1α | DSVNN (SEQ ID NO: 39) |
| | CDR2α | IPSGT (SEQ ID NO: 40) |
| | CDR3α | CAVSHGRGGATNKLIF (SEQ ID NO: 41) |
| | α | MKRILGALLGLLSAQVCCVRGIQVEQSPPDLILQEGANSTLRCNESDSVN |
| | variable | NLQWFHQNPWGQLINLFYIPSGTKQNGRLSATTVATERYSLLYISSSQTT |
| | domain | DSGVYFCAVSHGRGGATNKLIFGTGTLLAVQPN (SEQ ID NO: 42) |
| | α chain | MKRILGALLGLLSAQVCCVRGIQVEQSPPDLILQEGANSTLRCNFSDSVN |
| | with | NLQWFHQNPWGQLINLFYIPSGTKQNGRLSATTVATERYSLLYISSSQTT |
| | TRAC | DSGVYFCAVSHGRGGATNKLIFGTGTLLAVQPNIQNPDPAVYQLRDSKSS |
| | constant | DKSVCLFTDFDSQTNVSQSKDSDVYITDKCVLDMRSMDEKSNSAVAWSNK |
| | domain | SDFACANAFNNSIIPEDTFFPSPESSCDVKLVEKSFETDTNLNFQNLSVI |
| | | GFRILLLKVAGENLLMTLRLWSS (SEQ ID NO: 43) |
| beta | CDR1β | MNHNS (SEQ ID NO: 44) |
| | CDR2β | SASEGT (SEQ ID NO: 45) |
| | CDR3β | CASDRVLGYEQYF (SEQ ID NO: 46) |
| | β | MSIGLLCCVAFSLLWASPVNAGVTQTPKFQVLKTGQSMTLQCAQDMNHNS |
| | variable | MYWYRQDPGMGLRLIYYSASEGTTDKGEVPNGYNVSRLNKREFSLRLESA |
| | domain | APSQTSVYFCASDRVLGYEQYFGPGTRLTVTE (SEQ ID NO: 47) |
| | β chain | MSIGLLCCVAFSLLWASPVNAGVTQTPKFQVLKTGQSMTLQCAQDMNHNS |
| | with | MYWYRQDPGMGLRLIYYSASEGTTDKGEVPNGYNVSRLNKREFSLRLESA |
| | TRBC1 | APSQTSVYFCASDRVLGYEQYFGPGTRLTVTEDLNKVEPPEVAVFEPSEA |
| | constant | EISHTQKATLVCLATGFFPDHVELSWWVNGKEVHSGVSTDPQPLKEQPAL |
| | domain | NDSRYCLSSRLRVSATFWQNPRNHFRCQVQFYGLSENDEWTQDRAKPVTQ |
| | | IVSAEAWGRADCGFTSVSYQQGVLSATILYEILLGKATLYAVLVSALVLM |
| | | AMVKRKDF (SEQ ID NO: 48) |
| | β chain | MSIGLLCCVAFSLLWASPVNAGVTQTPKFQVLKTGQSMTLQCAQDMNHNS |
| | with | MYWYRQDPGMGLRLIYYSASEGTTDKGEVPNGYNVSRLNKREFSLRLESA |
| | TRBC2 | APSQTSVYFCASDRVLGYEQYFGPGTRLTVTDLKNVEPPEVAVFEPSEAE |
| | constant | ISHTQKATLVCLATGFYPDHVELSWWVNGKEVHSGVSTDPQPLKEQPALN |
| | domain | DSRYCLSSRLRVSATFWQNPRNHERCQVQFYGLSENDEWTQDRAKPVTQI |
| | | VSAEAWGRADCGFTSESYQQGVLSATILYEILLGKATLYAVLVSALVLMA |
| | | MVKRKDSRG (SEQ ID NO: 49) |

In one aspect, the present invention provides a TCR comprising:
(i) a CDR3α comprising or consisting of the amino acid sequence of CADWVDMRF (SEQ ID NO: 8) or a variant thereof having up to three amino acid substitutions, additions or deletions, and/or a CDR3β comprising or consisting of the amino acid sequence of CSAPLDRGSNQPQHF (SEQ ID NO: 13) or a variant thereof having up to three amino acid substitutions, additions or deletions;
(ii) a CDR3α comprising or consisting of the amino acid sequence of CAVSRPNSGYSTLTF (SEQ ID NO: 19) or a variant thereof having up to three amino acid substitutions, additions or deletions, and/or a CDR3β comprising or consisting of the amino acid sequence of CASSVRTPSGQETQYF (SEQ ID NO: 24) or a variant thereof having up to three amino acid substitutions, additions or deletions;
(iii) a CDR3α comprising or consisting of the amino acid sequence of CAVETGGGATNKLIF (SEQ ID NO: 30) or a variant thereof having up to three amino acid substitutions, additions or deletions, and/or a CDR3β comprising or consisting of the amino acid sequence of CASSEFWLTQETQYF (SEQ ID NO: 35) or a variant thereof having up to three amino acid substitutions, additions or deletions; or
(iv) a CDR3α comprising or consisting of the amino acid sequence of CAVSHGRGGATNKLIF (SEQ ID NO: 41) or a variant thereof having up to three amino acid substitutions, additions or deletions, and/or a CDR3β comprising or consisting of the amino acid sequence of CASDRVLGYEQYF (SEQ ID NO: 46) or a variant thereof having up to three amino acid substitutions, additions or deletions.

In some embodiments, the CDR3 variants have up to two amino acid substitutions, additions or deletions. In some embodiments, the CDR3 variants have up to one amino acid substitution, addition or deletion. In some embodiments, the CDR3 variants have up to three amino acid substitutions. In some embodiments, the CDR3 variants have up to two amino acid substitutions. In some embodiments, the CDR3 variants have up to one amino acid substitution.

In some embodiments, the TCR comprises:
(i) a CDR3α comprising or consisting of the amino acid sequence of CADWVDMRF (SEQ ID NO: 8) and/or a CDR3β comprising or consisting of the amino acid sequence of CSAPLDRGSNQPQHF (SEQ ID NO: 13);
(ii) a CDR3α comprising or consisting of the amino acid sequence of CAVSRPNSGYSTLTF (SEQ ID NO: 19) and/or a CDR3β comprising or consisting of the amino acid sequence of CASSVRTPSGQETQYF (SEQ ID NO: 24);
(iii) a CDR3α comprising or consisting of the amino acid sequence of CAVETGGGATNKLIF (SEQ ID NO: 30) and/or a CDR3β comprising or consisting of the amino acid sequence of CASSEFWLTQETQYF (SEQ ID NO: 35); or
(iv) a CDR3α comprising or consisting of the amino acid sequence of CAVSHGRGGATNKLIF (SEQ ID NO: 41) and/or a CDR3β comprising or consisting of the amino acid sequence of CASDRVLGYEQYF (SEQ ID NO: 46).

In some embodiments, the TCR further comprises:
(i) (a) a CDR1α comprising or consisting of the amino acid sequence of DSASNY (SEQ ID NO: 6), or a variant thereof having up to three amino acid substitutions, additions or deletions; and (b) a CDR2α comprising or consisting of the amino acid sequence of IRSNVGE (SEQ ID NO: 7), or a variant thereof having up to three amino acid substitutions, additions or deletions; and/or (a) a CDR1β comprising or consisting of the amino acid sequence of DFQATT (SEQ ID NO: 11), or a variant thereof having up to three amino acid substitutions, additions or deletions; and (b) a CDR2β comprising or consisting of the amino acid sequence of SNEGSKA (SEQ ID NO: 12), or a variant thereof having up to three amino acid substitutions, additions or deletions;
(ii) (a) a CDR1α comprising or consisting of the amino acid sequence of TSGFNG (SEQ ID NO: 17), or a variant thereof having up to three amino acid substitutions, additions or deletions; and (b) a CDR2α comprising or consisting of the amino acid sequence of NVLDGL (SEQ ID NO: 18), or a variant thereof having up to three amino acid substitutions, additions or deletions; and/or (a) a CDR1β comprising or consisting of the amino acid sequence of PRHDT (SEQ ID NO: 22), or a variant thereof having up to three amino acid substitutions, additions or deletions; and (b) a CDR2β comprising or consisting of the amino acid sequence of FYEKMQ (SEQ ID NO: 23), or a variant thereof having up to three amino acid substitutions, additions or deletions;
(iii) (a) a CDR1α comprising or consisting of the amino acid sequence of VSNAYN (SEQ ID NO: 28), or a variant thereof having up to three amino acid substitutions, additions or deletions; and (b) a CDR2α comprising or consisting of the amino acid sequence of GSKP (SEQ ID NO: 29), or a variant thereof having up to three amino acid substitutions, additions or deletions; and/or (a) a CDR1β comprising or consisting of the amino acid sequence of MNHNS (SEQ ID NO: 33), or a variant thereof having up to three amino acid substitutions, additions or deletions; and (b) a CDR2β comprising or consisting of the amino acid sequence of SASEGT (SEQ ID NO: 34), or a variant thereof having up to three amino acid substitutions, additions or deletions; or
(iv) (a) a CDR1α comprising or consisting of the amino acid sequence of DSVNN (SEQ ID NO: 39), or a variant thereof having up to three amino acid substitutions, additions or deletions; and (b) a CDR2α comprising or consisting of the amino acid sequence of IPSGT (SEQ ID NO: 40), or a variant thereof having up to three amino acid substitutions, additions or deletions; and/or (a) a CDR1β comprising or consisting of the amino acid sequence of MNHNS (SEQ ID NO: 44), or a variant thereof having up to three amino acid substitutions, additions or deletions; and (b) a CDR2β comprising or consisting of the amino acid sequence of SASEGT (SEQ ID NO: 45), or a variant thereof having up to three amino acid substitutions, additions or deletions.

In some embodiments, the TCR comprises:
(i) an α chain variable domain comprising or consisting of the amino acid sequence of SEQ ID NO: 9 or a variant thereof having at least 70%, at least 75%, at least 80%, at least 85%, at least 90%, at least 95%, at least 96%, at least 97%, at least 98%, or at least 99% sequence identity thereto; and/or a β chain variable domain comprising or consisting of the amino acid sequence of SEQ ID NO: 14 or a variant thereof having at least 70%, at least 75%, at least 80%, at least 85%, at least 90%, at least 95%, at least 96%, at least 97%, at least 98%, or at least 99% sequence identity thereto;
(ii) an α chain variable domain comprising or consisting of the amino acid sequence of SEQ ID NO: 20 or a variant thereof having at least 70%, at least 75%, at least 80%, at least 85%, at least 90%, at least 95%, at least 96%, at least 97%, at least 98%, or at least 99% sequence identity thereto; and/or a β chain variable domain comprising or consisting of the amino acid sequence of SEQ ID NO: 25 or a variant thereof having at least 70%, at least 75%, at least 80%, at least 85%, at least 90%, at least 95%, at least 96%, at least 97%, at least 98%, or at least 99% sequence identity thereto;
(iii) an α chain variable domain comprising or consisting of the amino acid sequence of SEQ ID NO: 31 or a variant thereof having at least 70%, at least 75%, at least 80%, at least 85%, at least 90%, at least 95%, at least 96%, at least 97%, at least 98%, or at least 99% sequence identity thereto; and/or a β chain variable domain comprising or consisting of the amino acid sequence of SEQ ID NO: 36 or a variant thereof having at least 70%, at least 75%, at least 80%, at least 85%, at least 90%, at least 95%, at least 96%, at least 97%, at least 98%, or at least 99% sequence identity thereto; or
(iv) an α chain variable domain comprising or consisting of the amino acid sequence of SEQ ID NO: 42 or a variant thereof having at least 70%, at least 75%, at least 80%, at least 85%, at least 90%, at least 95%, at least 96%, at least 97%, at least 98%, or at least 99% sequence identity thereto; and/or a β chain variable domain comprising or consisting of the amino acid sequence of SEQ ID NO: 47 or a variant thereof having at least 70%, at least 75%, at least 80%, at least 85%, at least 90%, at least 95%, at least 96%, at least 97%, at least 98%, or at least 99% sequence identity thereto

In some embodiments, the TCR comprises:
(i) an α chain comprising or consisting of the amino acid sequence of SEQ ID NO: 10 or a variant thereof having at least 70%, at least 75%, at least 80%, at least 85%, at least 90%, at least 95%, at least 96%, at least 97%, at least 98%, or at least 99% sequence identity thereto; and/or a β chain comprising or consisting of the amino acid sequence of SEQ ID NO: 15 or 16, or variants thereof having at least 70%, at least 75%, at least 80%, at least 85%, at least 90%, at least 95%, at least 96%, at least 97%, at least 98%, or at least 99% sequence identity thereto;
(ii) an α chain comprising or consisting of the amino acid sequence of SEQ ID NO: 21 or a variant thereof having at least 70%, at least 75%, at least 80%, at least 85%, at least 90%, at least 95%, at least 96%, at least 97%, at least 98%, or at least 99% sequence identity thereto; and/or a β chain comprising or consisting of the amino acid sequence of SEQ ID NO: 26 or 27, or variants thereof having at least 70%, at least 75%, at least 80%, at least 85%, at least 90%, at least 95%, at least 96%, at least 97%, at least 98%, or at least 99% sequence identity thereto;
(iii) an α chain comprising or consisting of the amino acid sequence of SEQ ID NO: 32 or a variant thereof having at least 70%, at least 75%, at least 80%, at least 85%, at least 90%, at least 95%, at least 96%, at least 97%, at least 98%, or at least 99% sequence identity thereto; and/or a β chain comprising or consisting of the amino acid sequence of SEQ ID NO: 37 or 38, or variants thereof having at least 70%, at least 75%, at least 80%, at least 85%, at least 90%, at least 95%, at least 96%, at least 97%, at least 98%, or at least 99% sequence identity thereto; or
(iv) an α chain comprising or consisting of the amino acid sequence of SEQ ID NO: 43 or a variant thereof having at least 70%, at least 75%, at least 80%, at least 85%, at least 90%, at least 95%, at least 96%, at least 97%, at least 98%, or at least 99% sequence identity thereto; and/or a β chain comprising or consisting of the amino acid sequence of SEQ ID NO: 48 or 49, or variants thereof having at least 70%, at least 75%, at least 80%, at least 85%, at least 90%, at least 95%, at least 96%, at least 97%, at least 98%, or at least 99% sequence identity thereto.

In one aspect, the present invention provides a TCR comprising:
(i) (a) a CDR1α comprising or consisting of the amino acid sequence of DSASNY (SEQ ID NO: 6), or a variant thereof having up to three amino acid substitutions, additions or deletions; (b) a CDR2α comprising or consisting of the amino acid sequence of IRSNVGE (SEQ ID NO: 7), or a variant thereof having up to three amino acid substitutions, additions or deletions; (c) a CDR3α comprising or consisting of the amino acid sequence of CADWVDMRF (SEQ ID NO: 8), or a variant thereof having up to three amino acid substitutions, additions or deletions; and/or (a) a CDR1β comprising or consisting of the amino acid sequence of DFQATT (SEQ ID NO: 11), or a variant thereof having up to three amino acid substitutions, additions or deletions; (b) a CDR2β comprising or consisting of the amino acid sequence of SNEGSKA (SEQ ID NO: 12), or a variant thereof having up to three amino acid substitutions, additions or deletions; (c) a CDR3β comprising or consisting of the amino acid sequence of CSAPLDRGSNQPQHF (SEQ ID NO: 13), or a variant thereof having up to three amino acid substitutions, additions or deletions;
(ii) (a) a CDR1α comprising or consisting of the amino acid sequence of TSGFNG (SEQ ID NO: 17), or a variant thereof having up to three amino acid substitutions, additions or deletions; (b) a CDR2α comprising or consisting of the amino acid sequence of NVLDGL (SEQ ID NO: 18), or a variant thereof having up to three amino acid substitutions, additions or deletions; (c) a CDR3α comprising or consisting of the amino acid sequence of CAVSRPNSGYSTLTF (SEQ ID NO: 19), or a variant thereof having up to three amino acid substitutions, additions or deletions; and/or (a) a CDR1β comprising or consisting of the amino acid sequence of PRHDT (SEQ ID NO: 22), or a variant thereof having up to three amino acid substitutions, additions or deletions; (b) a CDR2β comprising or consisting of the amino acid sequence of FYEKMQ (SEQ ID NO: 23), or a variant thereof having up to three amino acid substitutions, additions or deletions; (c) a CDR3β comprising or consisting of the amino acid sequence of CASSVRTPSGQETQYF (SEQ ID NO: 24), or a variant thereof having up to three amino acid substitutions, additions or deletions;
(iii) (a) a CDR1α comprising or consisting of the amino acid sequence of VSNAYN (SEQ ID NO: 28), or a variant thereof having up to three amino acid substitutions, additions or deletions; (b) a CDR2α comprising or consisting of the amino acid sequence of GSKP (SEQ ID NO: 29), or a variant thereof having up to three amino acid substitutions, additions or deletions; (c) a CDR3α comprising or consisting of the amino acid sequence of CAVETGGGATNKLIF (SEQ ID NO: 30), or a variant thereof having up to three amino acid substitutions, additions or deletions; and/or (a) a CDR1β comprising or consisting of the amino acid sequence of MNHNS (SEQ ID NO: 33), or a variant thereof having up to three amino acid substitutions, additions or deletions; (b) a CDR2β comprising or consisting of the amino acid sequence of SASEGT (SEQ ID NO: 34), or a variant thereof having up to three amino acid substitutions, additions or deletions; (c) a CDR3β comprising or consisting of the amino acid sequence of CASSEFWLTQETQYF (SEQ ID NO: 35), or a variant thereof having up to three amino acid substitutions, additions or deletions; or
(iv) (a) a CDR1α comprising or consisting of the amino acid sequence of DSVNN (SEQ ID NO: 39), or a variant thereof having up to three amino acid substitutions, additions or deletions; (b) a CDR2α comprising or consisting of the amino acid sequence of IPSGT (SEQ ID NO: 40), or a variant thereof having up to three amino acid substitutions, additions or deletions; (c) a CDR3α comprising or consisting of the amino acid sequence of CAVSHGRGGATNKLIF (SEQ ID NO: 41), or a variant thereof having up to three amino acid substitutions, additions or deletions; and/or (a) a CDR1β comprising or consisting of the amino acid sequence of MNHNS (SEQ ID NO: 44), or a variant thereof having up to three amino acid substitutions, additions or deletions; (b) a CDR2β comprising or consisting of the amino acid sequence of SASEGT (SEQ ID NO: 45), or a variant thereof having up to three amino acid substitutions, additions or deletions; (c) a CDR3β comprising or consisting of the amino acid sequence of CASDRVLGYEQYF (SEQ ID NO: 46), or a variant thereof having up to three amino acid substitutions, additions or deletions.

In some embodiments, the CDR variants have up to two amino acid substitutions, additions or deletions. In some embodiments, the CDR variants have up to one amino acid substitution, addition or deletion. In some embodiments, the CDR variants have up to three amino acid substitutions. In some embodiments, the CDR variants have up to two amino acid substitutions. In some embodiments, the CDR variants have up to one amino acid substitution.

In some embodiments, the TCR comprises:
(i) (a) a CDR1α comprising or consisting of the amino acid sequence of DSASNY (SEQ ID NO: 6); (b) a CDR2α comprising or consisting of the amino acid sequence of IRSNVGE (SEQ ID NO: 7); (c) a CDR3α comprising or consisting of the amino acid sequence of CADWVDMRF (SEQ ID NO: 8); and/or (a) a CDR1β comprising or consisting of the amino acid sequence of DFQATT (SEQ ID NO: 11); (b) a CDR2β comprising or consisting of the amino acid sequence of SNEGSKA (SEQ ID NO: 12); (c) a CDR3β comprising or consisting of the amino acid sequence of CSAPLDRGSNQPQHF (SEQ ID NO: 13);
(ii) (a) a CDR1α comprising or consisting of the amino acid sequence of TSGFNG (SEQ ID NO: 17); (b) a CDR2α comprising or consisting of the amino acid sequence of NVLDGL (SEQ ID NO: 18); (c) a CDR3α comprising or consisting of the amino acid sequence of CAVSRPNSGYSTLTF (SEQ ID NO: 19); and/or (a) a CDR1β comprising or consisting of the amino acid sequence of PRHDT (SEQ ID NO: 22); (b) a CDR2β comprising or consisting of the amino acid sequence of FYEKMQ (SEQ ID NO: 23); (c) a CDR3β comprising or consisting of the amino acid sequence of CASSVRTPSGQETQYF (SEQ ID NO: 24);
(iii) (a) a CDR1α comprising or consisting of the amino acid sequence of VSNAYN (SEQ ID NO: 28); (b) a CDR2α comprising or consisting of the amino acid sequence of GSKP (SEQ ID NO: 29); (c) a CDR3α comprising or consisting of the amino acid sequence of CAVETGGGATNKLIF (SEQ ID NO: 30); and/or (a) a CDR1β comprising or consisting of the amino acid sequence of MNHNS (SEQ ID NO: 33); (b) a CDR2β comprising or consisting of the amino acid sequence of SASEGT (SEQ ID NO: 34); (c) a CDR3β comprising or consisting of the amino acid sequence of CASSEFWLTQETQYF (SEQ ID NO: 35); or
(iv) (a) a CDR1α comprising or consisting of the amino acid sequence of DSVNN (SEQ ID NO: 39); (b) a CDR2α comprising or consisting of the amino acid sequence of IPSGT (SEQ ID NO: 40); (c) a CDR3α comprising or consisting of the amino acid sequence of CAVSHGRGGATNKLIF (SEQ ID NO: 41); and/or (a) a CDR1β comprising or consisting of the amino acid sequence of MNHNS (SEQ ID NO: 44); (b) a CDR2β comprising or consisting of the amino acid sequence of SASEGT (SEQ ID NO: 45); (c) a CDR3β comprising or consisting of the amino acid sequence of CASDRVLGYEQYF (SEQ ID NO: 46).

In one aspect, the present invention provides a TCR comprising:
(i) an α chain variable domain comprising or consisting of the amino acid sequence of SEQ ID NO: 9 or a variant thereof having at least 70%, at least 75%, at least 80%, at least 85%, at least 90%, at least 95%, at least 96%, at least 97%, at least 98%, or at least 99% sequence identity thereto; and/or a β chain variable domain comprising or consisting of the amino acid sequence of SEQ ID NO: 14 or a variant thereof having at least 70%, at least 75%, at least 80%, at least 85%, at least 90%, at least 95%, at least 96%, at least 97%, at least 98%, or at least 99% sequence identity thereto;
(ii) an α chain variable domain comprising or consisting of the amino acid sequence of SEQ ID NO: 20 or a variant thereof having at least 70%, at least 75%, at least 80%, at least 85%, at least 90%, at least 95%, at least 96%, at least 97%, at least 98%, or at least 99% sequence identity thereto; and/or a β chain variable domain comprising or consisting of the amino acid sequence of SEQ ID NO: 25 or a variant thereof having at least 70%, at least 75%, at least 80%, at least 85%, at least 90%, at least 95%, at least 96%, at least 97%, at least 98%, or at least 99% sequence identity thereto;
(iii) an α chain variable domain comprising or consisting of the amino acid sequence of SEQ ID NO: 31 or a variant thereof having at least 70%, at least 75%, at least 80%, at least 85%, at least 90%, at least 95%, at least 96%, at least 97%, at least 98%, or at least 99% sequence identity thereto; and/or a β chain variable domain comprising or consisting of the amino acid sequence of SEQ ID NO: 36 or a variant thereof having at least 70%, at least 75%, at least 80%, at least 85%, at least 90%, at least 95%, at least 96%, at least 97%, at least 98%, or at least 99% sequence identity thereto; or
(iv) an α chain variable domain comprising or consisting of the amino acid sequence of SEQ ID NO: 42 or a variant thereof having at least 70%, at least 75%, at least 80%, at least 85%, at least 90%, at least 95%, at least 96%, at least 97%, at least 98%, or at least 99% sequence identity thereto; and/or a β chain variable domain comprising or consisting of the amino acid sequence of SEQ ID NO: 47 or a variant thereof having at least 70%, at least 75%, at least 80%, at least 85%, at least 90%, at least 95%, at least 96%, at least 97%, at least 98%, or at least 99% sequence identity thereto.

In one aspect, the present invention provides a TCR comprising an α chain variable domain comprising or consisting of the amino acid sequence of SEQ ID NO: 9 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises a CDR3α comprising or consisting of the amino acid sequence of CADWVDMRF (SEQ ID NO: 8) or a variant thereof having up to three amino acid substitutions, additions or deletions; and/or a β chain variable domain comprising or consisting of the amino acid sequence of SEQ ID NO: 14 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises a CDR3β comprising or consisting of the amino acid sequence of CSAPLDRGSNQPQHF (SEQ ID NO: 13) or a variant thereof having up to three amino acid substitutions, additions or deletions.

In one aspect, the present invention provides a TCR comprising an α chain variable domain comprising or consisting of the amino acid sequence of SEQ ID NO: 20 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises a CDR3α comprising or consisting of the amino acid sequence of CAVSRPNSGYSTLTF (SEQ ID NO: 19) or a variant thereof having up to three amino acid substitutions, additions or deletions; and/or a β chain variable domain comprising or consisting of the amino acid sequence of SEQ ID NO: 25 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises a CDR3β comprising or consisting of the amino acid sequence of CASSVRTPSGQETQYF (SEQ ID NO: 24) or a variant thereof having up to three amino acid substitutions, additions or deletions.

In one aspect, the present invention provides a TCR comprising an α chain variable domain comprising or consisting of the amino acid sequence of SEQ ID NO: 31 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises a CDR3α comprising or consisting of the amino acid sequence of CAVETGGGATNKLIF (SEQ ID NO: 30) or a variant thereof having up to three amino acid substitutions, additions or deletions; and/or a β chain variable domain comprising or consisting of the amino acid sequence of SEQ ID NO: 36 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises a CDR3β comprising or consisting of the amino acid sequence of CASSEFWLTQETQYF (SEQ ID NO: 35) or a variant thereof having up to three amino acid substitutions, additions or deletions.

In one aspect, the present invention provides a TCR comprising an α chain variable domain comprising or consisting of the amino acid sequence of SEQ ID NO: 42 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises a CDR3α comprising or consisting of the amino acid sequence of CAVSHGRGGATNKLIF (SEQ ID NO: 41) or a variant thereof having up to three amino acid substitutions, additions or deletions; and/or a β chain variable domain comprising or consisting of the amino acid sequence of SEQ ID NO: 47 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises a CDR3β comprising or consisting of the amino acid sequence of CASDRVLGYEQYF (SEQ ID NO: 46) or a variant thereof having up to three amino acid substitutions, additions or deletions.

In one aspect, the present invention provides a TCR comprising an α chain variable domain comprising or consisting of the amino acid sequence of SEQ ID NO: 9 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises (a) a CDR1α comprising or consisting of the amino acid sequence of DSASNY (SEQ ID NO: 6), or a variant thereof having up to three amino acid substitutions, additions or deletions; (b) a CDR2α comprising or consisting of the amino acid sequence of IRSNVGE (SEQ ID NO: 7), or a variant thereof having up to three amino acid substitutions, additions or deletions; (c) a CDR3α comprising or consisting of the amino acid sequence of CADWVDMRF (SEQ ID NO: 8) or a variant thereof having up to three amino acid substitutions, additions or deletions; and/or a β chain variable domain comprising or consisting of the amino acid sequence of SEQ ID NO: 14 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises (a) a CDR1β comprising or consisting of the amino acid sequence of DFQATT (SEQ ID NO: 11), or a variant thereof having up to three amino acid substitutions, additions or deletions; (b) a CDR2β comprising or consisting of the amino acid sequence of SNEGSKA (SEQ ID NO: 12), or a variant thereof having up to three amino acid substitutions, additions or deletions; (c) a CDR3β comprising or consisting of the amino acid sequence of CSAPLDRGSNQPQHF (SEQ ID NO: 13), or a variant thereof having up to three amino acid substitutions, additions or deletions.

In one aspect, the present invention provides a TCR comprising an α chain variable domain comprising or consisting of the amino acid sequence of SEQ ID NO: 20 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises (a) a CDR1α comprising or consisting of the amino acid sequence of TSGFNG (SEQ ID NO: 17), or a variant thereof having up to three amino acid substitutions, additions or deletions; (b) a CDR2α comprising or consisting of the amino acid sequence of NVLDGL (SEQ ID NO: 18), or a variant thereof having up to three amino acid substitutions, additions or deletions; (c) a CDR3α comprising or consisting of the amino acid sequence of CAVSRPNSGYSTLTF (SEQ ID NO: 19) or a variant thereof having up to three amino acid substitutions, additions or deletions; and/or a β chain variable domain comprising or consisting of the amino acid sequence of SEQ ID NO: 25 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises (a) a CDR1β comprising or consisting of the amino acid sequence of PRHDT (SEQ ID NO: 22), or a variant thereof having up to three amino acid substitutions, additions or deletions; (b) a CDR2β comprising or consisting of the amino acid sequence of FYEKMQ (SEQ ID NO: 23), or a variant thereof having up to three amino acid substitutions, additions or deletions; (c) a CDR3β comprising or consisting of the amino acid sequence of CASSVRTPSGQETQYF (SEQ ID NO: 24), or a variant thereof having up to three amino acid substitutions, additions or deletions.

In one aspect, the present invention provides a TCR comprising an α chain variable domain comprising or consisting of the amino acid sequence of SEQ ID NO: 31 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises (a) a CDR1α comprising or consisting of the amino acid sequence of VSNAYN (SEQ ID NO: 28), or a variant thereof having up to three amino acid substitutions, additions or deletions; (b) a CDR2α comprising or consisting of the amino acid sequence of GSKP (SEQ ID NO: 29), or a variant thereof having up to three amino acid substitutions, additions or deletions; (c) a CDR3α comprising or consisting of the amino acid sequence of CAVETGGGATNKLIF (SEQ ID NO: 30) or a variant thereof having up to three amino acid substitutions, additions or deletions; and/or a β chain variable domain comprising or consisting of the amino acid sequence of SEQ ID NO: 36 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises (a) a CDR13 comprising or consisting of the amino acid sequence of MNHNS (SEQ ID NO: 33), or a variant thereof having up to three amino acid substitutions, additions or deletions; (b) a CDR2β comprising or consisting of the amino acid sequence of SASEGT (SEQ ID NO: 34), or a variant thereof having up to three amino acid substitutions, additions or deletions; (c) a CDR3β comprising or consisting of the amino acid sequence of CASSEFWLTQETQYF (SEQ ID NO: 35), or a variant thereof having up to three amino acid substitutions, additions or deletions.

In one aspect, the present invention provides a TCR comprising an α chain variable domain comprising or consisting of the amino acid sequence of SEQ ID NO: 42 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises (a) a CDR1α comprising or consisting of the amino acid sequence of DSVNN (SEQ ID NO: 39), or a variant thereof having up to three amino acid substitutions, additions or deletions; (b) a CDR2α comprising or consisting of the amino acid sequence of IPSGT (SEQ ID NO: 40), or a variant thereof having up to three amino acid substitutions, additions or deletions; (c) a CDR3α comprising or consisting of the amino acid sequence of CAVSHGRGGATNKLIF (SEQ ID NO: 41) or a variant thereof having up to three amino acid substitutions, additions or deletions; and/or a β chain variable domain comprising or consisting of the amino acid sequence of SEQ ID NO: 47 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises (a) a CDR1β comprising or consisting of the amino acid sequence of MNHNS (SEQ ID NO: 44), or a variant thereof having up to three amino acid substitutions, additions or deletions; (b) a CDR2β comprising or consisting of the amino acid sequence of SASEGT (SEQ ID NO: 45), or a variant thereof having up to three amino acid substitutions, additions or deletions; (c) a CDR3β comprising or consisting of the amino acid sequence of CASDRVLGYEQYF (SEQ ID NO: 46), or a variant thereof having up to three amino acid substitutions, additions or deletions.

In one aspect, the present invention provides a TCR comprising:
(i) an α chain comprising or consisting of the amino acid sequence of SEQ ID NO: 10 or a variant thereof having at least 70%, at least 75%, at least 80%, at least 85%, at least 90%, at least 95%, at least 96%, at least 97%, at least 98%, or at least 99% sequence identity thereto; and/or a β chain comprising or consisting of the amino acid sequence of SEQ ID NO: 15 or 16, or variants thereof having at least 70%, at least 75%, at least 80%, at least 85%, at least 90%, at least 95%, at least 96%, at least 97%, at least 98%, or at least 99% sequence identity thereto;
(ii) an α chain comprising or consisting of the amino acid sequence of SEQ ID NO: 21 or a variant thereof having at least 70%, at least 75%, at least 80%, at least 85%, at least 90%, at least 95%, at least 96%, at least 97%, at least 98%, or at least 99% sequence identity thereto; and/or a β chain comprising or consisting of the amino acid sequence of SEQ ID NO: 26 or 27, or variants thereof having at least 70%, at least 75%, at least 80%, at least 85%, at least 90%, at least 95%, at least 96%, at least 97%, at least 98%, or at least 99% sequence identity thereto;
(iii) an α chain comprising or consisting of the amino acid sequence of SEQ ID NO: 32 or a variant thereof having at least 70%, at least 75%, at least 80%, at least 85%, at least 90%, at least 95%, at least 96%, at least 97%, at least 98%, or at least 99% sequence identity thereto; and/or a β chain comprising or consisting of the amino acid sequence of SEQ ID NO: 37 or 38, or variants thereof having at least 70%, at least 75%, at least 80%, at least 85%, at least 90%, at least 95%, at least 96%, at least 97%, at least 98%, or at least 99% sequence identity thereto; or
(iv) an α chain comprising or consisting of the amino acid sequence of SEQ ID NO: 43 or a variant thereof having at least 70%, at least 75%, at least 80%, at least 85%, at least 90%, at least 95%, at least 96%, at least 97%, at least 98%, or at least 99% sequence identity thereto; and/or a β chain comprising or consisting of the amino acid sequence of SEQ ID NO: 48 or 49, or variants thereof having at least 70%, at least 75%, at least 80%, at least 85%, at least 90%, at least 95%, at least 96%, at least 97%, at least 98%, or at least 99% sequence identity thereto.

In one aspect, the present invention provides a TCR comprising an α chain comprising or consisting of the amino acid sequence of SEQ ID NO: 10 or a variant thereof having at least 70%, at least 75%, at least 80%, at least 85%, at least 90%, at least 95%, at least 96%, at least 97%, at least 98%, or at least 99% sequence identity thereto, wherein the α chain comprises a CDR3α comprising or consisting of the amino acid sequence of CADWVDMRF (SEQ ID NO: 8) or a variant thereof having up to three amino acid substitutions, additions or deletions; and/or a β chain comprising or consisting of the amino acid sequence of SEQ ID NO: 15 or 16, or variants thereof having at least 70%, at least 75%, at least 80%, at least 85%, at least 90%, at least 95%, at least 96%, at least 97%, at least 98%, or at least 99% sequence identity thereto, wherein the β chain comprises a CDR3β comprising or consisting of the amino acid sequence of CSAPLDRGSNQPQHF (SEQ ID NO: 13) or a variant thereof having up to three amino acid substitutions, additions or deletions.

In one aspect, the present invention provides a TCR comprising an α chain comprising or consisting of the amino acid sequence of SEQ ID NO: 21 or a variant thereof having at least 70%, at least 75%, at least 80%, at least 85%, at least 90%, at least 95%, at least 96%, at least 97%, at least 98%, or at least 99% sequence identity thereto, wherein the α chain comprises a CDR3α comprising or consisting of the amino acid sequence of CAVSRPNSGYSTLTF (SEQ ID NO: 19) or a variant thereof having up to three amino acid substitutions, additions or deletions; and/or a β chain comprising or consisting of the amino acid sequence of SEQ ID NO: 26 or 27, or variants thereof having at least 70%, at least 75%, at least 80%, at least 85%, at least 90%, at least 95%, at least 96%, at least 97%, at least 98%, or at least 99% sequence identity thereto, wherein the β chain comprises a CDR3β comprising or consisting of the amino acid sequence of CASSVRTPSGQETQYF (SEQ ID NO: 24) or a variant thereof having up to three amino acid substitutions, additions or deletions.

In one aspect, the present invention provides a TCR comprising an α chain comprising or consisting of the amino acid sequence of SEQ ID NO: 32 or a variant thereof having at least 70%, at least 75%, at least 80%, at least 85%, at least 90%, at least 95%, at least 96%, at least 97%, at least 98%, or at least 99% sequence identity thereto, wherein the α chain comprises a CDR3α comprising or consisting of the amino acid sequence of CAVETGGGATNKLIF (SEQ ID NO: 30) or a variant thereof having up to three amino acid substitutions, additions or deletions; and/or a β chain comprising or consisting of the amino acid sequence of SEQ ID NO: 37 or 38, or variants thereof having at least 70%, at least 75%, at least 80%, at least 85%, at least 90%, at least 95%, at least 96%, at least 97%, at least 98%, or at least 99% sequence identity thereto, wherein the β chain comprises a CDR3β comprising or consisting of the amino acid sequence of CASSEFWLTQETQYF (SEQ ID NO: 35) or a variant thereof having up to three amino acid substitutions, additions or deletions.

In one aspect, the present invention provides a TCR comprising an α chain comprising or consisting of the amino acid sequence of SEQ ID NO: 43 or a variant thereof having at least 70%, at least 75%, at least 80%, at least 85%, at least 90%, at least 95%, at least 96%, at least 97%, at least 98%, or at least 99% sequence identity thereto, wherein the α chain comprises a CDR3α comprising or consisting of the amino acid sequence of CAVSHGRGGATNKLIF (SEQ ID NO: 41) or a variant thereof having up to three amino acid substitutions, additions or deletions; and/or a β chain comprising or consisting of the amino acid sequence of SEQ ID NO: 48 or 49, or variants thereof having at least 70%, at least 75%, at least 80%, at least 85%, at least 90%, at least 95%, at least 96%, at least 97%, at least 98%, or at least 99% sequence identity thereto, wherein the β chain comprises a CDR3β comprising or consisting of the amino acid sequence of CASDRVLGYEQYF (SEQ ID NO: 46) or a variant thereof having up to three amino acid substitutions, additions or deletions.

In one aspect, the present invention provides a TCR comprising an α chain comprising or consisting of the amino acid sequence of SEQ ID NO: 10 or a variant thereof having at least 70%, at least 75%, at least 80%, at least 85%, at least 90%, at least 95%, at least 96%, at least 97%, at least 98%, or at least 99% sequence identity thereto, wherein the α chain comprises (a) a CDR1α comprising or consisting of the amino acid sequence of DSASNY (SEQ ID NO: 6), or a variant thereof having up to three amino acid substitutions, additions or deletions; (b) a CDR2α comprising or consisting of the amino acid sequence of IRSNVGE (SEQ ID NO: 7), or a variant thereof having up to three amino acid substitutions, additions or deletions; (c) a CDR3α comprising or consisting of the amino acid sequence of CADWVDMRF (SEQ ID NO: 8) or a variant thereof having up to three amino acid substitutions, additions or deletions; and/or a β chain comprising or consisting of the amino acid sequence of SEQ ID NO: 15 or 16, or variants thereof having at least 70%, at least 75%, at least 80%, at least 85%, at least 90%, at least 95%, at least 96%, at least 97%, at least 98%, or at least 99% sequence identity thereto, wherein the β chain comprises (a) a CDR1β comprising or consisting of the amino acid sequence of DFQATT (SEQ ID NO: 11), or a variant thereof having up to three amino acid substitutions, additions or deletions; (b) a CDR2β comprising or consisting of the amino acid sequence of SNEGSKA (SEQ ID NO: 12), or a variant thereof having up to three amino acid substitutions, additions or deletions; (c) a CDR3β comprising or consisting of the amino acid sequence of CSAPLDRGSNQPQHF (SEQ ID NO: 13), or a variant thereof having up to three amino acid substitutions, additions or deletions.

In one aspect, the present invention provides a TCR comprising an α chain comprising or consisting of the amino acid sequence of SEQ ID NO: 21 or a variant thereof having at least 70%, at least 75%, at least 80%, at least 85%, at least 90%, at least 95%, at least 96%, at least 97%, at least 98%, or at least 99% sequence identity thereto, wherein the α chain comprises (a) a CDR1α comprising or consisting of the amino acid sequence of TSGFNG (SEQ ID NO: 17), or a variant thereof having up to three amino acid substitutions, additions or deletions; (b) a CDR2α comprising or consisting of the amino acid sequence of NVLDGL (SEQ ID NO: 18), or a variant thereof having up to three amino acid substitutions, additions or deletions; (c) a CDR3α comprising or consisting of the amino acid sequence of CAVSRPNSGYSTLTF (SEQ ID NO: 19) or a variant thereof having up to three amino acid substitutions, additions or deletions; and/or a β chain comprising or consisting of the amino acid sequence of SEQ ID NO: 26 or 27, or variants thereof having at least 70%, at least 75%, at least 80%, at least 85%, at least 90%, at least 95%, at least 96%, at least 97%, at least 98%, or at least 99% sequence identity thereto, wherein the β chain comprises (a) a CDR1β comprising or consisting of the amino acid sequence of PRHDT (SEQ ID NO: 22), or a variant thereof having up to three amino acid substitutions, additions or deletions; (b) a CDR2β comprising or consisting of the amino acid sequence of FYEKMQ (SEQ ID NO: 23), or a variant thereof having up to three amino acid substitutions, additions or deletions; (c) a CDR3β comprising or consisting of the amino acid sequence of CASSVRTPSGQETQYF (SEQ ID NO: 24), or a variant thereof having up to three amino acid substitutions, additions or deletions.

In one aspect, the present invention provides a TCR comprising an α chain comprising or consisting of the amino acid sequence of SEQ ID NO: 32 or a variant thereof having at least 70%, at least 75%, at least 80%, at least 85%, at least 90%, at least 95%, at least 96%, at least 97%, at least 98%, or at least 99% sequence identity thereto, wherein the α chain comprises (a) a CDR1α comprising or consisting of the amino acid sequence of VSNAYN (SEQ ID NO: 28), or a variant thereof having up to three amino acid substitutions, additions or deletions; (b) a CDR2α comprising or consisting of the amino acid sequence of GSKP (SEQ ID NO: 29), or a variant thereof having up to three amino acid substitutions, additions or deletions; (c) a CDR3α comprising or consisting of the amino acid sequence of CAVETGGGATNKLIF (SEQ ID NO: 30) or a variant thereof having up to three amino acid substitutions, additions or deletions; and/or a β chain comprising or consisting of the amino acid sequence of SEQ ID NO: 37 or 38, or variants thereof having at least 70%, at least 75%, at least 80%, at least 85%, at least 90%, at least 95%, at least 96%, at least 97%, at least 98%, or at least 99% sequence identity thereto, wherein the β chain comprises (a) a CDR1β comprising or consisting of the amino acid sequence of MNHNS (SEQ ID NO: 33), or a variant thereof having up to three amino acid substitutions, additions or deletions; (b) a CDR2β comprising or consisting of the amino acid sequence of SASEGT (SEQ ID NO: 34), or a variant thereof having up to three amino acid substitutions, additions or deletions; (c) a CDR3β comprising or consisting of the amino acid sequence of CASSEFWLTQETQYF (SEQ ID NO: 35), or a variant thereof having up to three amino acid substitutions, additions or deletions.

In one aspect, the present invention provides a TCR comprising an α chain comprising or consisting of the amino acid sequence of SEQ ID NO: 43 or a variant thereof having at least 70%, at least 75%, at least 80%, at least 85%, at least 90%, at least 95%, at least 96%, at least 97%, at least 98%, or at least 99% sequence identity thereto, wherein the α chain comprises (a) a CDR1α comprising or consisting of the amino acid sequence of DSVNN (SEQ ID NO: 39), or a variant thereof having up to three amino acid substitutions, additions or deletions; (b) a CDR2α comprising or consisting of the amino acid sequence of IPSGT (SEQ ID NO: 40), or a variant thereof having up to three amino acid substitutions, additions or deletions; (c) a CDR3α comprising or consisting of the amino acid sequence of CAVSHGRGGATNKLIF (SEQ ID NO: 41) or a variant thereof having up to three amino acid substitutions, additions or deletions; and/or a β chain comprising or consisting of the amino acid sequence of SEQ ID NO: 48 or 49, or variants thereof having at least 70%, at least 75%, at least 80%, at least 85%, at least 90%, at least 95%, at least 96%, at least 97%, at least 98%, or at least 99% sequence identity thereto, wherein the β chain comprises (a) a CDR1ß comprising or consisting of the amino acid sequence of MNHNS (SEQ ID NO: 44), or a variant thereof having up to three amino acid substitutions, additions or deletions; (b) a CDR2β comprising or consisting of the amino acid sequence of SASEGT (SEQ ID NO: 45), or a variant thereof having up to three amino acid substitutions, additions or deletions; (c) a CDR3β comprising or consisting of the amino acid sequence of CASDRVLGYEQYF (SEQ ID NO: 46), or a variant thereof having up to three amino acid substitutions, additions or deletions.

### Survivin-Specific TCRs

In one aspect, the present invention provides a TCR which binds to a Survivin peptide when presented by an MHC.

### Survivin

Survivin is a protein that, in humans, is encoded by the BIRC5 gene and that is also called baculoviral inhibitor of apoptosis repeat-containing 5 (BIRC5).

Survivin has dual roles in promoting cell proliferation and preventing apoptosis. Surivin is a component of a chromosome passage protein complex (CPC) which is essential for chromosome alignment and segregation during mitosis and cytokinesis. Survivin may act as an important regulator of the localization of this complex. Although it is less clear how Survivin inhibits apoptosis, interactions with other members of the inhibitors of apoptosis (IAP) protein family appear to be important (see e.g. Wheatley, S. P. and Altieri, D. C., 2019. Journal of Cell Science, 132 (7), p.jcs223826).

BIRC5 has four exons and five introns, and encodes ten splice variants, seven with known function. The predominant wild-type form is referred to as Survivin, after which 2β and ΔEx3 are the most common forms. Both deviate from Survivin at the exon 2-exon 3 junction: 2β has a 26-aa insert that makes it pro-apoptotic; exon 3 is deleted in ΔEx3, causing a frameshift resulting in a different C-terminus.

The human Survivin protein may have the amino acid sequence set out in UniProt entry O15392. An example Survivin protein has the amino acid sequence set out below in SEQ ID NO: 50.

(SEQ ID NO: 50)
MGAPTLPPAWQPFLKDHRISTFKNWPFLEGCACTPERMAEA
GFIHCPTENEPDLAQCFFCFKELEGWEPDDDPIEEHKKHSS
GCAFLSVKKQFEELTLGEFLKLDRERAKNKIAKETNNKKKE
FEETAKKVRRAIEQLAAMD
Example Survivin protein

### Survivin Peptides

In one aspect, the invention provides a Survivin peptide. Suitably, the Survivin peptide is an isolated peptide.

As used herein, the term “Survivin peptide” may refer to a peptide comprising an amino acid sequence derived from a Survivin protein. Suitably, a Survivin peptide may comprise at least 5, at least 6, at least 7, at least 8, at least 9, at least 10, at least 11, at least 12, at least 13, at least 14, at least 15, at least 16, at least 17, at least 18, at least 19, at least 20, or at least 25 contiguous amino acid residues of a Survivin protein amino acid sequence.

The Survivin peptide may be an immunogenic peptide. The Survivin peptide may bind to any suitable MHC. Suitably, the Survivin peptide binds to an MHC encoded by a HLA-A, HLA-B or a HLA-C allele. Suitably, the Survivin peptide binds to an MHC encoded by a HLA-A allele. Suitably, Suitably, the Survivin peptide binds to an MHC encoded by a HLA-A*02 allele. Suitably, the Survivin peptide binds to an MHC encoded by HLA-A*0201.

An example Survivin peptide (see e.g. Bernatchez, C., et al., 2011. Vaccine, 29(16), pp. 3021-3030) has the amino acid sequence set out below in SEQ ID NO: 51.

(SEQ ID NO: 51)
LMLGEFLKL
Example Survivin peptide

The Survivin peptide may comprise or consist of the amino acid sequence of LMLGEFLKL (SEQ ID NO: 51) or a variant thereof having up to three amino acid substitutions, additions or deletions. In some embodiments, the Survivin peptide comprises or consists of the amino acid sequence of LMLGEFLKL (SEQ ID NO: 51) or a variant thereof having up to two amino acid substitutions, additions or deletions. In some embodiments, the Survivin peptide comprises or consists of the amino acid sequence of LMLGEFLKL (SEQ ID NO: 51) or a variant thereof having up to one amino acid substitution, addition or deletion. In some embodiments, the Survivin peptide comprises or consists of the amino acid sequence of LMLGEFLKL (SEQ ID NO: 51) or a variant thereof having up to three amino acid substitutions. In some embodiments, the Survivin peptide comprises or consists of the amino acid sequence of LMLGEFLKL (SEQ ID NO: 51) or a variant thereof having up to two amino acid substitutions. In some embodiments, the Survivin peptide comprises or consists of the amino acid sequence of LMLGEFLKL (SEQ ID NO: 51) or a variant thereof having up to one amino acid substitution. In some embodiments, the Survivin peptide comprises or consists of the amino acid sequence of LMLGEFLKL (SEQ ID NO: 51).

Another example Survivin peptide has the amino acid sequence set out below in SEQ ID NO: 88.

(SEQ ID NO: 88)
LTLGEFLKL
Example Survivin peptide

Various uses of the Survivin peptides described herein are contemplated by the invention.

For example, the Survivin peptides described herein may be administered to a subject, e.g. a human subject. Administration of the Survivin peptides of the invention may elicit an immune response against cells expressing or overexpressing Survivin protein, i.e. the Survivin peptide may be an immunogenic Survivin peptide.

The Survivin peptides described herein may be used to screen for and/or identify new TCR sequences which bind to Survivin cells. For example, T2 cells may be pulsed with a Survivin peptide mentioned in the invention and incubated with a T-cell population isolated from a donor. In this approach, expression of cytokines, e.g. CD107a and IFNγ, may be indicative of T-cells which recognise Survivin peptides.

### Survivin-Specific TCR Sequences

The present inventors have determined the amino acid sequences of a Survivin-specific TCR, including the CDR regions, which are responsible for binding specificity for the Survivin peptide.

The Survivin-specific TCR may be restricted to any suitable MHC (e.g. one or more HLA allele). Suitably, the Survivin-specific TCR is restricted to a HLA-A, HLA-B or a HLA-C allele. Suitably, the Survivin-specific TCR is restricted to a HLA-A allele. Suitably, the Survivin-specific TCR is restricted to a HLA-A*02 allele. Suitably, the Survivin-specific TCR is restricted to HLA-A*0201.

Example Survivin-specific TCR amino acid sequences are provided in the table below.

| Chain | Region | Example amino acid sequence |
|---|---|---|
| alpha | CDR1α | NYSPAY (SEQ ID NO: 52) |
| | CDR2α | IRENEKE (SEQ ID NO: 53) |
| | CDR3α | CALDRMDSSYKLIF (SEQ ID NO: 54) |
| | α | MESFLGGVLLILWLQVDWVKSQKIEQNSEALNIQEGKTATLTCNYTNYSPA |
| | variable | YLQWYRQDPGRGPVFLLLIRENEKEKRKERLKVTFDTTLKQSLFHITASQP |
| | domain | ADSATYLCALDRMDSSYKLIFGSGTRLLVRPD (SEQ ID NO: 55) |
| | α chain | MESFLGGVLLILWLQVDWVKSQKIEQNSEALNIQEGKTATLTCNYTNYSPA |
| | with | YLQWYRQDPGRGPVELLLIRENEKEKRKERLKVTFDTTLKQSLFHITASQP |
| | TRAC | ADSATYLCALDRMDSSYKLIFGSGTRLLVRPDIQNPDPAVYQLRDSKSSDK |
| | constant | SVCLFTDFDSQTNVSQSKDSDVYITDKTVLDMRSMDFKSNSAVAWSNKSDE |
| | domain | ACANAENNSIIPEDTFFPSPESSCDVKLVEKSFETDTNLNFQNLSVIGFRI |
| | | LLLKVAGENLLMTLRLWSS (SEQ ID NO: 56) |
| beta | CDR1β | MNHEY (SEQ ID NO: 57) |
| | CDR2β | SVGAGI (SEQ ID NO: 58) |
| | CDR3β | CASSYDQDGEAFF (SEQ ID NO: 59) |
| | β | MSIGLLCCAALSLLWAGPVNAGVTQTPKFQVLKTGQSMTLOCAQDMNHEYM |
| | variable | SWYRQDPGMGLRLIHYSVGAGITDQGEVPNGYNVSRSTTEDEPLRLLSAAP |
| | domain | SQTSVYFCASSYDQDGEAFFGQGTRLTVVE (SEQ ID NO: 60) |
| | β chain | MSIGLLCCAALSLLWAGPVNAGVTQTPKFQVLKTGQSMTLQCAQDMNHEYM |
| | with | SWYRQDPGMGLRLIHYSVGAGITDQGEVPNGYNVSRSTTEDFPLRLLSAAP |
| | TRBC1 | SQTSVYFCASSYDQDGEAFFGQGTRLTVVEDLNKVEPPEVAVFEPSEAEIS |
| | constant | HTQKATLVCLATGFFPDHVELSWWVNGKEVHSGVSTDPQPLKEQPALNDSR |
| | domain | YCLSSRLRVSATFWQNPRNHFRCQVQFYGLSENDEWTQDRAKPVTQIVSAE |
| | | AWGRADCGFTSVSYQQGVLSATILYEILLGKATLYAVLVSALVLMAMVKRK |
| | | DF (SEQ ID NO: 61) |
| | β chain | MSIGLLCCAALSLLWAGPVNAGVTQTPKFQVLKTGQSMTLQCAQDMNHEYM |
| | with | SWYRQDPGMGLRLIHYSVGAGITDQGEVPNGYNVSRSTTEDEPLRLLSAAP |
| | TRBC2 | SQTSVYFCASSYDQDGEAFFGQGTRLTVVEDLKNVEPPEVAVFEPSEAEIS |
| | constant | HTQKATLVCLATGFYPDHVELSWWVNGKEVHSGVSTDPQPLKEQPALNDSR |
| | domain | YCLSSRLRVSATFWQNPRNHERCQVQFYGLSENDEWTQDRAKPVTQIVSAE |
| | | AWGRADCGFTSESYQQGVLSATILYEILLGKATLYAVLVSALVLMAMVKRK |
| | | DSRG (SEQ ID NO: 62) |

In one aspect, the present invention provides a TCR comprising a CDR3α comprising or consisting of the amino acid sequence of CALDRMDSSYKLIF (SEQ ID NO: 54) or a variant thereof having up to three amino acid substitutions, additions or deletions, and/or a CDR3β comprising or consisting of the amino acid sequence of CASSYDQDGEAFF (SEQ ID NO: 59) or a variant thereof having up to three amino acid substitutions, additions or deletions.

In some embodiments, the TCR comprises a CDR3α comprising or consisting of the amino acid sequence of CALDRMDSSYKLIF (SEQ ID NO: 54) or a variant thereof having up to two amino acid substitutions, additions or deletions, and/or a CDR3β comprising or consisting of the amino acid sequence of CASSYDQDGEAFF (SEQ ID NO: 59) or a variant thereof 10 having up to two amino acid substitutions, additions or deletions. In some embodiments, the TCR comprises a CDR3α comprising or consisting of the amino acid sequence of CALDRMDSSYKLIF (SEQ ID NO: 54) or a variant thereof having up to one amino acid substitution, addition or deletion, and/or a CDR3β comprising or consisting of the amino acid sequence of CASSYDQDGEAFF (SEQ ID NO: 59) or a variant thereof having up to one amino acid substitution, addition or deletion. In some embodiments, the TCR comprises a CDR3α comprising or consisting of the amino acid sequence of CALDRMDSSYKLIF (SEQ ID NO: 54) or a variant thereof having up to three amino acid substitutions, and/or a CDR3β comprising or consisting of the amino acid sequence of CASSYDQDGEAFF (SEQ ID NO: 59) or a variant thereof having up to three amino acid substitutions. In some embodiments, the TCR comprises a CDR3α comprising or consisting of the amino acid sequence of CALDRMDSSYKLIF (SEQ ID NO: 54) or a variant thereof having up to two amino acid substitutions, and/or a CDR3β comprising or consisting of the amino acid sequence of CASSYDQDGEAFF (SEQ ID NO: 59) or a variant thereof having up to two amino acid substitutions. In some embodiments, the TCR comprises a CDR3α comprising or consisting of the amino acid sequence of CALDRMDSSYKLIF (SEQ ID NO: 54) or a variant thereof having up to one amino acid substitution, and/or a CDR3β comprising or consisting of the amino acid sequence of CASSYDQDGEAFF (SEQ ID NO: 59) or a variant thereof having up to one amino acid substitution. In some embodiments, the TCR comprises a CDR3α comprising or consisting of the amino acid sequence of CALDRMDSSYKLIF (SEQ ID NO: 54), and/or a CDR3β comprising or consisting of the amino acid sequence of CASSYDQDGEAFF (SEQ ID NO: 59).

In some embodiments, the TCR further comprises (a) a CDR1α comprising or consisting of the amino acid sequence of NYSPAY (SEQ ID NO: 52), or a variant thereof having up to three amino acid substitutions, additions or deletions; and (b) a CDR2α comprising or consisting of the amino acid sequence of IRENEKE (SEQ ID NO: 53), or a variant thereof having up to three amino acid substitutions, additions or deletions; and/or (a) a CDR1β comprising or consisting of the amino acid sequence of MNHEY (SEQ ID NO: 57), or a variant thereof having up to three amino acid substitutions, additions or deletions; and (b) a CDR2β comprising or consisting of the amino acid sequence of SVGAGI (SEQ ID NO: 58), or a variant thereof having up to three amino acid substitutions, additions or deletions.

In some embodiments, the TCR comprises an α chain variable domain comprising or consisting of the amino acid sequence of SEQ ID NO: 55 or a variant thereof having at least 70%, at least 75%, at least 80%, at least 85%, at least 90%, at least 95%, at least 96%, at least 97%, at least 98%, or at least 99% sequence identity thereto; and/or a β chain variable domain comprising or consisting of the amino acid sequence of SEQ ID NO: 60 or a variant thereof having at least 70%, at least 75%, at least 80%, at least 85%, at least 90%, at least 95%, at least 96%, at least 97%, at least 98%, or at least 99% sequence identity thereto.

In some embodiments, the TCR comprises an α chain comprising or consisting of the amino acid sequence of SEQ ID NO: 56 or a variant thereof having at least 70%, at least 75%, at least 80%, at least 85%, at least 90%, at least 95%, at least 96%, at least 97%, at least 98%, or at least 99% sequence identity thereto; and/or a β chain comprising or consisting of the amino acid sequence of SEQ ID NO: 61 or 62, or variants thereof having at least 70%, at least 75%, at least 80%, at least 85%, at least 90%, at least 95%, at least 96%, at least 97%, at least 98%, or at least 99% sequence identity thereto.

In one aspect, the present invention provides a TCR comprising (a) a CDR1α comprising or consisting of the amino acid sequence of NYSPAY (SEQ ID NO: 52), or a variant thereof having up to three amino acid substitutions, additions or deletions; (b) a CDR2α comprising or consisting of the amino acid sequence of IRENEKE (SEQ ID NO: 53), or a variant thereof having up to three amino acid substitutions, additions or deletions; (c) a CDR3α comprising or consisting of the amino acid sequence of CALDRMDSSYKLIF (SEQ ID NO: 54) or a variant thereof having up to three amino acid substitutions, additions or deletions; and/or (a) a CDR1β comprising or consisting of the amino acid sequence of MNHEY (SEQ ID NO: 57), or a variant thereof having up to three amino acid substitutions, additions or deletions; (b) a CDR2β comprising or consisting of the amino acid sequence of SVGAGI (SEQ ID NO: 58), or a variant thereof having up to three amino acid substitutions, additions or deletions; (c) a CDR3β comprising or consisting of the amino acid sequence of CASSYDQDGEAFF (SEQ ID NO: 59), or a variant thereof having up to three amino acid substitutions, additions or deletions.

In some embodiments, the TCR comprises (a) a CDR1α comprising or consisting of the amino acid sequence of NYSPAY (SEQ ID NO: 52), or a variant thereof having up to two amino acid substitutions, additions or deletions; (b) a CDR2α comprising or consisting of the amino acid sequence of IRENEKE (SEQ ID NO: 53), or a variant thereof having up to two amino acid substitutions, additions or deletions; (c) a CDR3α comprising or consisting of the amino acid sequence of CALDRMDSSYKLIF (SEQ ID NO: 54) or a variant thereof having up to two amino acid substitutions, additions or deletions; and/or (a) a CDR1β comprising or consisting of the amino acid sequence of MNHEY (SEQ ID NO: 57), or a variant thereof having up to two amino acid substitutions, additions or deletions; (b) a CDR2β comprising or consisting of the amino acid sequence of SVGAGI (SEQ ID NO: 58), or a variant thereof having up to two amino acid substitutions, additions or deletions; (c) a CDR3β comprising or consisting of the amino acid sequence of CASSYDQDGEAFF (SEQ ID NO: 59), or a variant thereof having up to two amino acid substitutions, additions or deletions.

In some embodiments, the TCR comprises (a) a CDR1α comprising or consisting of the amino acid sequence of NYSPAY (SEQ ID NO: 52), or a variant thereof having up to one amino acid substitution, addition or deletion; (b) a CDR2α comprising or consisting of the amino acid sequence of IRENEKE (SEQ ID NO: 53), or a variant thereof having up to one amino acid substitution, addition or deletion; (c) a CDR3α comprising or consisting of the amino acid sequence of CALDRMDSSYKLIF (SEQ ID NO: 54) or a variant thereof having up to one amino acid substitution, addition or deletion; and/or (a) a CDR1β comprising or consisting of the amino acid sequence of MNHEY (SEQ ID NO: 57), or a variant thereof having up to one amino acid substitution, addition or deletion; (b) a CDR2β comprising or consisting of the amino acid sequence of SVGAGI (SEQ ID NO: 58), or a variant thereof having up to one amino acid substitution, addition or deletion; (c) a CDR3β comprising or consisting of the amino acid sequence of CASSYDQDGEAFF (SEQ ID NO: 59), or a variant thereof having up to up to one amino acid substitution, addition or deletion.

In some embodiments, the TCR comprises (a) a CDR1α comprising or consisting of the amino acid sequence of NYSPAY (SEQ ID NO: 52); (b) a CDR2α comprising or consisting of the amino acid sequence of IRENEKE (SEQ ID NO: 53); (c) a CDR3α comprising or consisting of the amino acid sequence of CALDRMDSSYKLIF (SEQ ID NO: 54); and/or (a) a CDR1β comprising or consisting of the amino acid sequence of MNHEY (SEQ ID NO: 57); (b) a CDR2β comprising or consisting of the amino acid sequence of SVGAGI (SEQ ID NO: 58); (c) a CDR3β comprising or consisting of the amino acid sequence of CASSYDQDGEAFF (SEQ ID NO: 59).

In one aspect, the present invention provides a TCR comprising an α chain variable domain comprising or consisting of the amino acid sequence of SEQ ID NO: 55 or a variant thereof having at least 70%, at least 75%, at least 80%, at least 85%, at least 90%, at least 95%, at least 96%, at least 97%, at least 98%, or at least 99% sequence identity thereto; and/or a β chain variable domain comprising or consisting of the amino acid sequence of SEQ ID NO: 60 or a variant thereof having at least 70%, at least 75%, at least 80%, at least 85%, at least 90%, at least 95%, at least 96%, at least 97%, at least 98%, or at least 99% sequence identity thereto.

In one aspect, the present invention provides a TCR comprising an α chain variable domain comprising or consisting of the amino acid sequence of SEQ ID NO: 55 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises a CDR3α comprising or consisting of the amino acid sequence of CALDRMDSSYKLIF (SEQ ID NO: 54) or a variant thereof having up to three amino acid substitutions, additions or deletions; and/or a β chain variable domain comprising or consisting of the amino acid sequence of SEQ ID NO: 60 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises a CDR3β comprising or consisting of the amino acid sequence of CASSYDQDGEAFF (SEQ ID NO: 59), or a variant thereof having up to three amino acid substitutions, additions or deletions.

In one aspect, the present invention provides a TCR comprising an α chain variable domain comprising or consisting of the amino acid sequence of SEQ ID NO: 55 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises a CDR3α comprising or consisting of the amino acid sequence of CALDRMDSSYKLIF (SEQ ID NO: 54); and/or a β chain variable domain comprising or consisting of the amino acid sequence of SEQ ID NO: 60 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises a CDR3β comprising or consisting of the amino acid sequence of CASSYDQDGEAFF (SEQ ID NO: 59).

In one aspect, the present invention provides a TCR comprising an α chain variable domain comprising or consisting of the amino acid sequence of SEQ ID NO: 55 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises (a) a CDR1α comprising or consisting of the amino acid sequence of NYSPAY (SEQ ID NO: 52), or a variant thereof having up to three amino acid substitutions, additions or deletions; (b) a CDR2α comprising or consisting of the amino acid sequence of IRENEKE (SEQ ID NO: 53), or a variant thereof having up to three amino acid substitutions, additions or deletions; (c) a CDR3α comprising or consisting of the amino acid sequence of CALDRMDSSYKLIF (SEQ ID NO: 54) or a variant thereof having up to three amino acid substitutions, additions or deletions; and/or a β chain variable domain comprising or consisting of the amino acid sequence of SEQ ID NO: 60 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises (a) a CDR1β comprising or consisting of the amino acid sequence of MNHEY (SEQ ID NO: 57), or a variant thereof having up to three amino acid substitutions, additions or deletions; (b) a CDR2β comprising or consisting of the amino acid sequence of SVGAGI (SEQ ID NO: 58), or a variant thereof having up to three amino acid substitutions, additions or deletions; (c) a CDR3β comprising or consisting of the amino acid sequence of CASSYDODGEAFF (SEQ ID NO: 59), or a variant thereof having up to three amino acid substitutions, additions or deletions.

In one aspect, the present invention provides a TCR comprising an α chain variable domain comprising or consisting of the amino acid sequence of SEQ ID NO: 55 or a variant thereof having at least 70%, at least 75%, at least 80%, at least 85%, at least 90%, at least 95%, at least 96%, at least 97%, at least 98%, or at least 99% sequence identity thereto, wherein the α chain variable domain comprises (a) a CDR1α comprising or consisting of the amino acid sequence of NYSPAY (SEQ ID NO: 52); (b) a CDR2α comprising or consisting of the amino acid sequence of IRENEKE (SEQ ID NO: 53); (c) a CDR3α comprising or consisting of the amino acid sequence of CALDRMDSSYKLIF (SEQ ID NO: 54); and/or a β chain variable domain comprising or consisting of the amino acid sequence of SEQ ID NO: 60 or a variant thereof having at least 70%, at least 75%, at least 80%, at least 85%, at least 90%, at least 95%, at least 96%, at least 97%, at least 98%, or at least 99% sequence identity thereto, wherein the β chain variable domain comprises (a) a CDR1β comprising or consisting of the amino acid sequence of MNHEY (SEQ ID NO: 57); (b) a CDR2β comprising or consisting of the amino acid sequence of SVGAGI (SEQ ID NO: 58); (c) a CDR3β comprising or consisting of the amino acid sequence of CASSYDQDGEAFF (SEQ ID NO: 59).

In one aspect, the present invention provides a TCR comprising an α chain comprising or consisting of the amino acid sequence of SEQ ID NO: 56 or a variant thereof having at least 70%, at least 75%, at least 80%, at least 85%, at least 90%, at least 95%, at least 96%, at least 97%, at least 98%, or at least 99% sequence identity thereto; and/or a β chain comprising or consisting of the amino acid sequence of SEQ ID NO: 61 or 62, or variants thereof having at least 70%, at least 75%, at least 80%, at least 85%, at least 90%, at least 95%, at least 96%, at least 97%, at least 98%, or at least 99% sequence identity thereto.

In one aspect, the present invention provides a TCR comprising an α chain comprising or consisting of the amino acid sequence of SEQ ID NO: 56 or a variant thereof having at least 70%, at least 75%, at least 80%, at least 85%, at least 90%, at least 95%, at least 96%, at least 97%, at least 98%, or at least 99% sequence identity thereto, wherein the α chain comprises a CDR3α comprising or consisting of the amino acid sequence of CALDRMDSSYKLIF (SEQ ID NO: 54) or a variant thereof having up to three amino acid substitutions, additions or deletions; and/or a β chain comprising or consisting of the amino acid sequence of SEQ ID NO: 61 or 62, or variants thereof having at least 70%, at least 75%, at least 80%, at least 85%, at least 90%, at least 95%, at least 96%, at least 97%, at least 98%, or at least 99% sequence identity thereto, wherein the β chain comprises a CDR3β comprising or consisting of the amino acid sequence of CASSYDQDGEAFF (SEQ ID NO: 59), or a variant thereof having up to three amino acid substitutions, additions or deletions.

In one aspect, the present invention provides a TCR comprising an α chain comprising or consisting of the amino acid sequence of SEQ ID NO: 56 or a variant thereof having at least 70%, at least 75%, at least 80%, at least 85%, at least 90%, at least 95%, at least 96%, at least 97%, at least 98%, or at least 99% sequence identity thereto, wherein the α chain comprises a CDR3α comprising or consisting of the amino acid sequence of CALDRMDSSYKLIF (SEQ ID NO: 54); and/or a β chain comprising or consisting of the amino acid sequence of SEQ ID NO: 61 or 62, or variants thereof having at least 70%, at least 75%, at least 80%, at least 85%, at least 90%, at least 95%, at least 96%, at least 97%, at least 98%, or at least 99% sequence identity thereto, wherein the β chain comprises a CDR3β comprising or consisting of the amino acid sequence of CASSYDQDGEAFF (SEQ ID NO: 59).

In one aspect, the present invention provides a TCR comprising an α chain comprising or consisting of the amino acid sequence of SEQ ID NO: 56 or a variant thereof having at least 70%, at least 75%, at least 80%, at least 85%, at least 90%, at least 95%, at least 96%, at least 97%, at least 98%, or at least 99% sequence identity thereto, wherein the α chain comprises (a) a CDR1α comprising or consisting of the amino acid sequence of NYSPAY (SEQ ID NO: 52), or a variant thereof having up to three amino acid substitutions, additions or deletions; (b) a CDR2α comprising or consisting of the amino acid sequence of IRENEKE (SEQ ID NO: 53), or a variant thereof having up to three amino acid substitutions, additions or deletions; (c) a CDR3α comprising or consisting of the amino acid sequence of CALDRMDSSYKLIF (SEQ ID NO: 54) or a variant thereof having up to three amino acid substitutions, additions or deletions; and/or a β chain comprising or consisting of the amino acid sequence of SEQ ID NO: 61 or 62, or variants thereof having at least 70%, at least 75%, at least 80%, at least 85%, at least 90%, at least 95%, at least 96%, at least 97%, at least 98%, or at least 99% sequence identity thereto, wherein the β chain comprises (a) a CDR1β comprising or consisting of the amino acid sequence of MNHEY (SEQ ID NO: 57), or a variant thereof having up to three amino acid substitutions, additions or deletions; (b) a CDR2β comprising or consisting of the amino acid sequence of SVGAGI (SEQ ID NO: 58), or a variant thereof having up to three amino acid substitutions, additions or deletions; (c) a CDR3β comprising or consisting of the amino acid sequence of CASSYDQDGEAFF (SEQ ID NO: 59), or a variant thereof having up to three amino acid substitutions, additions or deletions.

In one aspect, the present invention provides a TCR comprising an α chain comprising or consisting of the amino acid sequence of SEQ ID NO: 56 or a variant thereof having at least 70%, at least 75%, at least 80%, at least 85%, at least 90%, at least 95%, at least 96%, at least 97%, at least 98%, or at least 99% sequence identity thereto, wherein the α chain comprises (a) a CDR1α comprising or consisting of the amino acid sequence of NYSPAY (SEQ ID NO: 52); (b) a CDR2α comprising or consisting of the amino acid sequence of IRENEKE (SEQ ID NO: 53); (c) a CDR3α comprising or consisting of the amino acid sequence of CALDRMDSSYKLIF (SEQ ID NO: 54); and/or a β chain comprising or consisting of the amino acid sequence of SEQ ID NO: 61 or 62, or variants thereof having at least 70%, at least 75%, at least 80%, at least 85%, at least 90%, at least 95%, at least 96%, at least 97%, at least 98%, or at least 99% sequence identity thereto, wherein the β chain comprises (a) a CDR1β comprising or consisting of the amino acid sequence of MNHEY (SEQ ID NO: 57); (b) a CDR2β comprising or consisting of the amino acid sequence of SVGAGI (SEQ ID NO: 58); (c) a CDR3β comprising or consisting of the amino acid sequence of CASSYDQDGEAFF (SEQ ID NO: 59).

In one aspect, the present invention provides a TCR comprising an α chain comprising or consisting of the amino acid sequence of SEQ ID NO: 56 or a variant thereof having at least 90%, at least 95%, at least 96%, at least 97%, at least 98%, or at least 99% sequence identity thereto, wherein the α chain comprises (a) a CDR1α comprising or consisting of the amino acid sequence of NYSPAY (SEQ ID NO: 52); (b) a CDR2α comprising or consisting of the amino acid sequence of IRENEKE (SEQ ID NO: 53); (c) a CDR3α comprising or consisting of the amino acid sequence of CALDRMDSSYKLIF (SEQ ID NO: 54); and a β chain comprising or consisting of the amino acid sequence of SEQ ID NO: 61 or 62, or variants thereof having at least 90%, at least 95%, at least 96%, at least 97%, at least 98%, or at least 99% sequence identity thereto, wherein the β chain comprises (a) a CDR1β comprising or consisting of the amino acid sequence of MNHEY (SEQ ID NO: 57); (b) a CDR2β comprising or consisting of the amino acid sequence of SVGAGI (SEQ ID NO: 58); (c) a CDR3β comprising or consisting of the amino acid sequence of CASSYDQDGEAFF (SEQ ID NO: 59).

### Reduced Mispairing and Improved TCR Expression

The TCR of the invention may be expressed in a T-cell to alter the antigen specificity of the T-cell. TCR-transduced T-cells may express at least two TCR alpha and two TCR beta chains. While the endogenous TCR alpha/beta chains form a receptor that is self-tolerant, the introduced TCR alpha/beta chains form a receptor with defined specificity for the given target antigen.

However, TCR gene therapy requires sufficient expression of transferred TCRs. Transferred TCR might be diluted by the presence of the endogeneous TCR, resulting in suboptimal expression of the tumor specific TCR. Furthermore, mispairing between endogenous and introduced chains may occur to form novel receptors, which might display unexpected specificities for self-antigens and cause autoimmune damage when transferred into patients.

Hence, several strategies have been explored to reduce the risk of mispairing between endogenous and introduced TCR chains. Mutations of the TCR alpha/beta interface is one strategy currently employed to reduce unwanted mispairing. For example, the introduction of a cysteine in the constant domains of the alpha and beta chain can allow the formation of a disulfide bond and enhance the pairing of the introduced chains while reducing mispairing with wild type chains.

Accordingly, the TCRs of the invention may comprise one or more mutations at the α chain/β chain interface, such that when the α chain and the β chain are expressed in a T-cell, the frequency of mispairing between said chains and endogenous TCR α and β chains is reduced. In some embodiments, the one or more mutations introduce a cysteine residue into the constant region domain of each of the α chain and the β chain, wherein the cysteine residues are capable of forming a disulphide bond between the α chain and the β chain. Such modification of TCRs is described in e.g. Boulter, J. M et al. (2003) Protein Engineering 16:707-711 and Kuball, L. et al. (2007) Blood 109:2331-8.

In some embodiments, the one or more mutations are at amino acid positions selected from those disclosed in Table 1 of Boulter, J. M et al. (2003) Protein Engineering 16:707-711. As described above, mutations of TCR constant domains disclosed herein may be described based on a numbering convention in which the first amino acid of each of SEQ ID NOs: 1-3 is assigned to be position 2. In some embodiments, the one or more mutations are a substitution of one or more of the following amino acids with cysteine:

| TRAC residue | TRBC residue |
|---|---|
| Threonine 48 | Serine 57 |
| Threonine 45 | Serine 77 |
| Serine 61 | Serine 57 |
| Leucine 50 | Serine 57 |
| Tyrosine 10 | Serine 17 |
| Serine 15 | Valine 13 |
| Serine 15 | Glutamate 15 |
| Threonine 45 | Aspartate 59 |
| Leucine 12 | Serine 17 |
| Serine 61 | Arginine 79 |
| Leucine 13 | Phenylalanine 14 |
| Valine 22 | Phenylalanine 14 |
| Tyrosine 43 | Leucine 63 |

In some embodiments, the TCR comprises one or more of the following groups of mutations:
(a) a substitution of threonine at position 48 of the TCR alpha constant domain with cysteine; and/or a substitution of serine at position 57 of the TCR beta constant domain with cysteine;
(b) a substitution of threonine at position 45 of the TCR alpha constant domain with cysteine; and/or a substitution of serine at position 77 of the TCR beta constant domain with cysteine;
(c) a substitution of serine at position 61 of the TCR alpha constant domain with cysteine; and/or a substitution of serine at position 57 of the TCR beta constant domain with cysteine;
(d) a substitution of leucine at position 50 of the TCR alpha constant domain with cysteine; and/or a substitution of serine at position 57 of the TCR beta constant domain with cysteine;
(e) a substitution of tyrosine at position 10 of the TCR alpha constant domain with cysteine; and/or a substitution of serine at position 17 of the TCR beta constant domain with cysteine;
(f) a substitution of serine at position 15 of the TCR alpha constant domain with cysteine; and/or a substitution of valine at position 13 of the TCR beta constant domain with cysteine;
(g) a substitution of serine at position 15 of the TCR alpha constant domain with cysteine; and/or a substitution of glutamate at position 15 of the TCR beta constant domain with cysteine;
(h) a substitution of threonine at position 45 of the TCR alpha constant domain with cysteine; and/or a substitution of aspartate at position 59 of the TCR beta constant domain with cysteine;
(i) a substitution of leucine 12 at position 48 of the TCR alpha constant domain with cysteine; and/or a substitution of serine at position 17 of the TCR beta constant domain with cysteine;
(j) a substitution of serine at position 61 of the TCR alpha constant domain with cysteine; and/or a substitution of arginine at position 79 of the TCR beta constant domain with cysteine;
(k) a substitution of leucine at position 12 of the TCR alpha constant domain with cysteine; and/or a substitution of phenylalanine at position 14 of the TCR beta constant domain with cysteine;
(l) a substitution of valine at position 22 of the TCR alpha constant domain with cysteine; and/or a substitution of phenylalanine at position 14 of the TCR beta constant domain with cysteine; and/or
(m) a substitution of tyrosine at position 43 of the TCR alpha constant domain with cysteine; and/or a substitution of leucine at position 63 of the TCR beta constant domain with cysteine.

In some embodiments, the TCR comprises a substitution of threonine at position 48 of the TCR alpha constant domain with cysteine; and/or a substitution of serine at position 57 of the TCR beta constant domain with cysteine.

Another strategy to reduce mispairing relies on the introduction of polynucleotide sequences encoding siRNA, added to the genes encoding for the tumor specific TCR α and or β chains, and designed to limit the expression of the endogenous TCR genes (see e.g. Okamoto S. Cancer research 69, 9003-9011, 2009). Suitably, the vector or polynucleotide encoding the TCRs of the invention may comprise one or more siRNA or other agents aimed at limiting or abrogating the expression of the endogenous TCR genes.

It is also possible to combine artificial nucleases, such as zinc finger nucleases (ZFN), transcription activator-like effector nucleases (TALEN) or CRISPR/Cas systems, designed to target the constant regions of the endogenous genes, e.g. TCR genes (TRAC and, or TRBC), to obtain the permanent disruption of the endogenous TCR alpha and/or beta chain genes, thus allowing full expression of the tumor specific TCR and thus reducing or abrogating the risk of TCR mispairing. This process, known as TCR gene editing, proved superior to TCR gene transfer in vitro and in vivo (see e.g. Provasi E., Genovese P., Nature Medicine May; 18(5):807-15; 2012; Mastaglio S. et al. (2017) Blood 130:606-618; and Ruggiero, E., et al., 2022. Science Translational Medicine, 14(631)). Suitably, the TCRs of the invention may be used to edit T cell specificity by TCR disruption and genetic addition of the tumor specific TCR.

In addition, the genome editing technology allows targeted integration of an expression cassette, comprising a polynucleotide encoding a TCR of the invention, and optionally one or more promoter regions and/or other expression control sequences, into an endogenous gene disrupted by the artificial nucleases (see e.g. Lombardo A., Nature biotechnology 25, 1298-1306; 2007). Suitably, the TCRs of the invention may be used to edit T-cell specificity by targeted integration of a polynucleotide encoding a TCR of the invention at a genomic region. The integration may be targeted by an artificial nuclease.

A cell, such as a T cell, may therefore be genetically engineered to comprise a TCR of the invention. In addition, a cell, such as a T cell, may be genetically edited by gene disruption, for example TRAC and/or TRBC disruption obtained by, for example, CRISPR/Cas9, or by targeted integration, for example of an expression cassette into an endogenous gene (such as an endogenous gene involved in antigen specificity, persistence, expansion, activity, resistance to exhaustion/senescence/inhibitory signals, homing capacity or other T-cell functions).

Any suitable TCR gene-editing agents may be used to genetically engineer a cell, such as a T cell. For example, a RNA-guided gene editing system comprising a guide RNA and a RNA-guided nuclease may be used to specifically introduce a double strand break (DSB) into an endogenous gene (e.g. TRAC and/or TRBC or a gene encoding an inhibitory receptor such as CD39, PD-1, LAG-3, Tim-3, or 2B4). Subsequently, a nucleotide sequence insert encoding a TCR of the invention can be introduced at a DSB site by homology-directed repair (HDR). A CRISPR/Cas9 system is an example of a commonly used RNA-guided gene editing system, but other RNA-guided gene editing systems may also be used.

Any suitable guide RNA may be used. A “guide RNA” (gRNA) confers target sequence specificity to a RNA-guided nuclease. Guide RNAs are non-coding short RNA sequences which bind to the complementary target DNA sequences. For example, in the CRISPR/Cas9 system, gRNA first binds to the Cas9 enzyme and the gRNA sequence guides the resulting complex via base-pairing to a specific location on the DNA, where Cas9 performs its nuclease activity by cutting the target DNA strand.

The guide RNA may comprise a trans-activating CRISPR RNA (tracrRNA) that provides the stem loop structure and a target-specific CRISPR RNA (crRNA) designed to cleave the gene target site of interest. The tracrRNA and crRNA may be annealed, for example by heating them at 95° C. for 5 minutes and letting them slowly cool down to room temperature for 10 minutes. Alternatively, the guide RNA may be a single guide RNA (sgRNA) that consists of both the crRNA and tracrRNA as a single construct.

The guide RNA may comprise of a 3′-end, which forms a scaffold for nuclease binding, and a 5′-end which is programmable to target different DNA sites. For example, the targeting specificity of CRISPR-Cas9 may be determined by the 15-25 bp sequence at the 5′ end of the guide RNA. The desired target sequence typically precedes a protospacer adjacent motif (PAM) which is a short DNA sequence usually 2-6 bp in length that follows the DNA region targeted for cleavage by the CRISPR system, such as CRISPR-Cas9. The PAM is required for a Cas nuclease to cut and is typically found 3-4 bp downstream from the cut site. After base pairing of the guide RNA to the target, Cas9 mediates a double strand break about 3-nt upstream of PAM.

Numerous tools exist for designing guide RNAs (e.g. Cui, Y., et al., 2018. Interdisciplinary Sciences: Computational Life Sciences, 10(2), pp. 455-465). For example, COSMID is a web-based tool for identifying and validating guide RNAs (Cradick T J, et al. Mol Ther-Nucleic Acids. 2014; 3(12):e214).

A list of exemplary guide sequences for use in the present invention is provided in the table below. The term “guide sequence” may refer to a sequence within a gRNA that recognizes, e.g., is complementary to, a target sequence, e.g. in a gene, that directs the gRNA to the target sequence for cleavage by a nuclease. A “guide sequence” may also be indicated as a “targeting sequence,” or a “spacer sequence.

| Example gRNA guide sequence | Genomic coordinates (hg38) |
|---|---|
| GAGUCACAUUCUCUAUGGUC (SEQ ID NO: 89) | chr5: 157106717-157106736 |
| AAUGUGGCAACGUGGUGCUC (SEQ ID NO: 90) | chr5: 157106817-157106836 |
| CUAAAUGGGGAUUUCCGCAA (SEQ ID NO: 91) | chr5: 157106752-157106771 |
| AUCCCCAUUUAGCCAGUAUC (SEQ ID NO: 92) | chr5: 157106760-157106779 |
| CUCUCUGCCGAGUCGGUGCA (SEQ ID NO: 93) | chr5: 157104717-157104736 |
| GUGAAGUCUCUCUGCCGAGU (SEQ ID NO: 94) | chr5: 157104710-157104729 |
| UGCCCCAUGCAUAGUUACCU (SEQ ID NO: 95) | chr5: 157104648-157104667 |
| AGGUCACCCCUGCACCGACU (SEQ ID NO: 96) | chr5: 157104727-157104746 |
| UGGCCCAGGUAACUAUGCAU (SEQ ID NO: 97) | chr5: 157104654-157104673 |
| CCAAGGAUGCUUACCACCAG (SEQ ID NO: 98) | chr5: 157104680-157104699 |
| CACCGCGGCGCGGUACUCGC (SEQ ID NO: 99) | chr12: 6773924-6773943 |
| GCUCAGCACCGUGUAGCGGC (SEQ ID NO: 100) | chr12: 6773780-6773799 |
| ACCUUCGUCUGUAUGCUGUU (SEQ ID NO: 101) | chr1: 160841820-160841839 |
| ACCAAACAGCAUACAGACGA (SEQ ID NO: 102) | chr1: 160841824-160841843 |
| AGUUGAGAAACCCCGCCUAC (SEQ ID NO: 103) | chr1: 160841462-160841481 |
| GUUGAGAAACCCCGCCUACA (SEQ ID NO: 104) | chr1: 160841461-160841480 |
| UUGAGAAACCCCGCCUACAG (SEQ ID NO: 105) | chr1: 160841460-160841479 |
| UGUAGCACCGCCCAGACGAC (SEQ ID NO: 106) | chr2: 241858792-241858811 |
| CGUCUGGGCGGUGCUACAAC (SEQ ID NO: 107) | chr2: 241858790-241858809 |
| AGGUGCCGCUGUCAUUGCGC (SEQ ID NO: 108) | chr2: 241852696-241852715 |
| CCUGCUCGUGGUGACCGAAG (SEQ ID NO: 109) | chr2: 241852918-241852937 |
| AAGGAUGGCUAGGAUAUUCU (SEQ ID NO: 110) | chr10: 95823264-95823283 |
| AAGAAUAUCCUAGCCAUCCU (SEQ ID NO: 111) | chr10: 95823263-95823282 |
| GAGAAUCAAAAUCGGUGAAU (SEQ ID NO: 112) | chr14: 22547576-22547595 |
| UCUCCGAGAGCCCGUAGAAC (SEQ ID NO: 113) | chr7: 142791989-142792008 |
| | chr7: 142801336-142801355 |
| CAAACACAGCGACCUCGGGU (SEQ ID NO: 114) | chr7: 142791716-142791735 |
| UGACAGCGGAAGUGGUUGCG (SEQ ID NO: 115) | chr7: 142791963-142791982 |
| | chr7: 142801310-142801329 |
| AAGUGAAGAGUUGGCAGACA (SEQ ID NO: 116) | chr10: 95844482-95844501 |
| AACUACCCCUUUGACUUCCA (SEQ ID NO: 117) | chr10: 95844534-95844553 |
| UCUGCUGGGCAAAUUCAGUC (SEQ ID NO: 118) | chr10: 95844614-95844633 |
| CUCUCAGCUGGUACACGGCA (SEQ ID NO: 119) | chr14: 22547525-22547544 |
| ACAAAACUGUGCUAGACAUG (SEQ ID NO: 120) | chr14: 22547641-22547660 |

In some embodiments, the present invention provides a cell, such as a T cell, that has been genetically engineered to insert a polynucleotide encoding a TCR of the present invention, using one or more guide RNA comprising a guide sequence selected from any of SEQ ID NOs: 89-120, or variants thereof having at least 90% sequence identity or at least 95% sequence identity thereto. A cell of the present invention may comprise a polynucleotide encoding a TCR of the present invention at any of the genomic coordinates listed in the table above.

In some embodiments, the present invention provides a cell, such as a T cell, that has been genetically engineered to insert a polynucleotide encoding a TCR of the present invention, using: (i) one or more guide RNA comprising a guide sequence selected from any of SEQ ID NOs: 89-98, or variants thereof having at least 90% sequence identity or at least 95% sequence identity thereto; (ii) one or more guide RNA comprising a guide sequence selected from any of SEQ ID NOs: 99-111, or variants thereof having at least 90% sequence identity or at least 95% sequence identity thereto; and optionally (iii) one or more guide RNA comprising a guide sequence selected from any of SEQ ID NOs: 112-115, or variants thereof having at least 90% sequence identity or at least 95% sequence identity thereto.

In some embodiments, the present invention provides a cell, such as a T cell, that has been genetically engineered to insert a polynucleotide encoding a TCR of the present invention, using: (i) one or more guide RNA comprising a guide sequence selected from any of SEQ ID NOs: 89-98, or variants thereof having at least 90% sequence identity or at least 95% sequence identity thereto; (ii) one or more guide RNA comprising a guide sequence selected from any of SEQ ID NOs: 101-105, or variants thereof having at least 90% sequence identity or at least 95% sequence identity thereto.

In some embodiments, the present invention provides a cell, such as a T cell, that has been genetically engineered to insert a polynucleotide encoding a TCR of the present invention, using: (i) a guide RNA comprising the guide sequence of SEQ ID NO: 89, or a variant thereof having at least 90% sequence identity or at least 95% sequence identity thereto; (ii) a guide RNA comprising the guide sequence of SEQ ID NO: 103, or a variant thereof having at least 90% sequence identity or at least 95% sequence identity thereto; and optionally (iii) a guide RNA comprising the guide sequence of SEQ ID NO: 114, or a variant thereof having at least 90% sequence identity or at least 95% sequence identity thereto.

Any suitable RNA-guided nuclease may be used. An “RNA-guided nuclease” is a nuclease which can be directed to a specific site by a guide RNA. RNA-guided nucleases include, but are not limited to, Type II CRISPR nucleases such as Cas9, and Type V CRISPR nucleases such as Cas12a and Cas12b, as well as other nucleases derived therefrom. Suitably, the RNA-guided nuclease is a Type II CRISPR nuclease, for example a Cas9 nuclease. The RNA-guided nuclease may be in a complex with the guide RNA, i.e. the guide RNA and the RNA-guided nuclease may together form a ribonucleoprotein (RNP). Suitably, the RNP is a Cas9 RNP. A RNP may be formed by any method known in the art, for example by incubating a RNA-guided nuclease with a guide RNA for 5-30 minutes at room temperature.

Another strategy developed to increase expression of the transferred TCR and to reduce TCR mispairing is murinization, which replaces the human TCR α and TCR β constant regions (e.g. the TRAC, TRBC1 and TRBC2 regions) by their murine counterparts. Murizination of TCR constant regions is described in, for example, Sommermeyer and Uckert J Immunol; 2010 (184:6223-6231). Suitably, the TCRs of the invention may be murinized.

### Polynucleotides

In one aspect, the present invention provides a polynucleotide encoding a TCR of the invention or a part thereof, such as the α chain and/or the β chain, a variable domain or a portion thereof.

The polynucleotide may be double or single stranded, and may be RNA or DNA. The polynucleotide may be an isolated polynucleotide.

It will be understood by a skilled person that numerous different polynucleotides can encode the same polypeptide as a result of the degeneracy of the genetic code. In addition, it is to be understood that the skilled person may, using routine techniques, make nucleotide substitutions, additions or deletions that do not affect the polypeptide sequence encoded by the polynucleotides of the invention to reflect the codon usage of any particular host organism in which the polypeptides of the invention are to be expressed.

Polynucleotides such as DNA polynucleotides may be produced recombinantly, synthetically, or by any means available to those of skill in the art. They may also be cloned by standard techniques. Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

The polynucleotides described herein may be modified by any method available in the art. Such modifications may be carried out in order to enhance the in vivo activity or lifespan of the polynucleotides of the invention.

hTERT-Specific TCR Sequences

Examples of hTERT-specific TCR nucleotide sequences are provided in the tables below.

The present invention also encompasses the TCR amino acid sequences encoded by the TCR nucleotide sequences provided in the tables below, and variants thereof.

hTERT #1

| Region | Example nucleotide sequence |
|---|---|
| α chain | atgacatccattcgagctgtatttatattcctgtggctgcagctggacttggtgaatggaga |
| with | gaatgtggagcagcatccttcaaccctgagtgtccaggagggagacagcgctgttatcaagt |
| TRAC | gtacttattcagacagtgcctcaaactacttcccttggtataagcaagaacttggaaaaaga |
| constant | cctcagcttattatagacattcgttcaaatgtgggcgaaaagaaagaccaacgaattgctgt |
| domain | tacattgaacaagacagccaaacatttctccctgcacatcacagagacccaacctgaagact |
| | cggctgtctacttctgtgcagactgggttgacatgcgctttggagcagggaccagactgaca |
| | gtaaaaccaaatatccagaaccctgaccctgccgtgtaccagctgagagactctaaatccag |
| | tgacaagtctgtctgcctattcaccgattttgattctcaaacaaatgtgtcacaaagtaagg |
| | attctgatgtgtatatcacagacaaaactgtgctagacatgaggtctatggacttcaagagc |
| | aacagtgctgtggcctggagcaacaaatctgactttgcatgtgcaaacgccttcaacaacag |
| | cattattccagaagacaccttcttccccagcccagaaagttcctgtgatgtcaagctggtcg |
| | agaaaagctttgaaacagatacgaacctaaactttcaaaacctgtcagtgattgggttccga |
| | atcctcctcctgaaagtggccgggtttaatctgctcatgacgctgcggctgtggtccagctg |
| | a (SEQ ID NO: 63) |
| β chain | atgctgctgcttctgctgcttctggggccaggtataagcctccttctacctgggagcttggc |
| with | aggctccgggcttggtgctgtcgtctctcaacatccgagctgggttatctgtaagagtggaa |
| TRBC1 | cctctgtgaagatcgagtgccgttccctggactttcaggccacaactatgttttggtatcgt |
| constant | cagttcccgaaacagagtctcatgctgatggcaacttccaatgagggctccaaggccacata |
| domain | cgagcaaggcgtcgagaaggacaagtttctcatcaaccatgcaagcctgaccttgtccactc |
| | tgacagtgaccagtgcccatcctgaagacagcagcttctacatctgcagtgctcctctcgac |
| | cgggggagcaatcagccccagcattttggtgatgggactcgactctccatcctagaggacct |
| | gaacaaggtgttcccacccgaggtcgctgtgtttgagccatcagaagcagagatctcccaca |
| | cccaaaaggccacactggtgtgcctggccacaggcttcttccccgaccacgtggagctgagc |
| | tggtgggtgaatgggaaggaggtgcacagtggggtcagcacggacccgcagcccctcaagga |
| | gcagcccgccctcaatgactccagatactgcctgagcagccgcctgagggtctcggccacct |
| | tctggcagaacccccgcaaccacttccgctgtcaagtccagttctacgggctctcggagaat |
| | gacgagtggacccaggatagggccaaacccgtcacccagatcgtcagcgccgaggcctgggg |
| | tagagcagactgtggctttacctcggtgtcctaccagcaaggggtcctgtctgccaccatcc |
| | tctatgagatcctgctagggaaggccaccctgtatgctgtgctggtcagcgcccttgtgttg |
| | atggccatggtcaagagaaaggatttctga (SEQ ID NO: 64) |
| β chain | atgctgctgcttctgctgcttctggggccaggtataagcctccttctacctgggagcttggc |
| with | aggctccgggcttggtgctgtcgtctctcaacatccgagctgggttatctgtaagagtggaa |
| TRBC2 | cctctgtgaagatcgagtgccgttccctggactttcaggccacaactatgttttggtatcgt |
| constant | cagttcccgaaacagagtctcatgctgatggcaacttccaatgagggctccaaggccacata |
| domain | cgagcaaggcgtcgagaaggacaagtttctcatcaaccatgcaagcctgaccttgtccactc |
| | tgacagtgaccagtgcccatcctgaagacagcagcttctacatctgcagtgctcctctcgac |
| | cgggggagcaatcagccccagcattttggtgatgggactcgactctccatcctagaggacct |
| | gaaaaacgtgttcccacccgaggtcgctgtgtttgagccatcagaagcagagatctcccaca |
| | cccaaaaggccacactggtgtgcctggccacaggcttctaccccgaccacgtggagctgagc |
| | tggtgggtgaatgggaaggaggtgcacagtggggtcagcacagacccgcagcccctcaagga |
| | gcagcccgccctcaatgactccagatactgcctgagcagccgcctgagggtctcggccacct |
| | tctggcagaacccccgcaaccacttccgctgtcaagtccagttctacgggctctcggagaat |
| | gacgagtggacccaggatagggccaaacctgtcacccagatcgtcagcgccgaggcctgggg |
| | tagagcagactgtggcttcacctccgagtcttaccagcaaggggtcctgtctgccaccatcc |
| | tctatgagatcttgctagggaaggccaccttgtatgccgtgctggtcagtgccctcgtgctg |
| | atggccatggtcaagagaaaggattccagaggctaa (SEQ ID NO: 65) |
| α chain | atgaccagcatcagagccgtgttcatcttcctgtggctgcagctggacctggtcaacggcga |
| with | gaatgtggaacagcaccccagcacactgagcgtgcaagagggcgattctgccgtgatcaagt |
| TRAC | gcacctacagcgacagcgccagcaactacttcccctggtacaagcaagagctgggcaaaaga |
| constant | ccccagctgatcatcgacatccggtccaacgtgggcgagaagaaggaccagagaatcgccgt |
| domain | gacactgaacaagaccgccaagcacttcagcctgcacatcaccgagacacagcctgaggata |
| | gcgccgtgtacttctgcgccgattgggtcgacatgagattcggagccggcaccagactgacc |
| | gtgaagcccaacattcagaaccccgatcctgccgtgtaccagctgagagacagcaagagcag |
| | cgacaagagcgtgtgcctgttcaccgacttcgacagccagaccaacgtgtcccagagcaagg |
| | acagcgacgtgtacatcacagataagtgcgtgctggacatgcggagcatggacttcaagagc |
| | aacagcgccgtggcctggtccaacaagtccgatttcgcctgcgccaacgccttcaacaacag |
| | cattatccccgaggacacattcttcccaagtcctgagagcagctgcgacgtgaagctggtgg |
| | aaaagagcttcgagacagacaccaacctgaacttccagaacctgagcgtgatcggcttccgg |
| | atcctgctgctgaaagtggccggcttcaacctgctgatgaccctgagactgtggtcctcctg |
| | a (SEQ ID NO: 66) |
| β chain | atgttgcttttgctgcttctgctcggccctggcatctctttgctgctgcctggttctctggc |
| with | cggctctggacttggagctgtggtgtctcagcacccctcttgggtcatctgcaagagcggca |
| TRBC2 | ccagcgtgaagatcgagtgcagaagcctggacttccaggccaccaccatgttctggtacaga |
| constant | cagttccccaagcagagcctgatgctgatggccacctctaacgagggcagcaaggccacata |
| domain | tgagcagggcgtcgagaaggacaagttcctgatcaaccacgccagcctgacactgagcaccc |
| | tgacagtgacaagcgcccatcctgaggacagcagcttctacatctgtagcgcccctctggac |
| | cggggctctaatcagcctcagcactttggcgacggcaccagactgagcatcctggaagatct |
| | gaagaacgtgttcccacctgaggtggccgtgttcgagccttctgaggccgagatcagccaca |
| | cacagaaagccacactcgtgtgtctggccaccggcttctatcccgatcacgtcgaactgtct |
| | tggtgggtcaacggcaaagaggtgcacagcggcgtctgtaccgatcctcagcctctgaaaga |
| | gcagcccgctctgaacgacagcagatactgcctgagcagcagactgagagtgtccgccacct |
| | tctggcagaaccccagaaaccacttcaggtgccaggtgcagttctacggcctgagcgagaac |
| | gatgagtggacccaggatagagccaagcctgtgacacagatcgtgtctgccgaagcctgggg |
| | cagagccgattgtggctttaccagcgagagctaccagcaaggcgtgctgtctgccaccatcc |
| | tgtacgagatcctgctgggcaaagccactctgtacgccgtgctggtgtctgccctggtcctg |
| | atggctatggtcaagagaaaggacagccggggctga (SEQ ID NO: 67) |

hTERT #2

| Region | Example nucleotide sequence |
|---|---|
| α chain | atgtggggagttttccttctttatgtttccatgaagatgggaggcactacaggacaaaacat |
| with | tgaccagcccactgagatgacagctacggaaggtgccattgtccagatcaactgcacgtacc |
| TRAC | agacatctgggttcaacgggctgttctggtaccagcaacatgctggcgaagcacccacattt |
| constant | ctgtcttacaatgttctggatggtttggaggagaaaggtcgtttttcttcattccttagtcg |
| domain | gtctaaagggtacagttacctccttttgaaggagctccagatgaaagactctgcctcttacc |
| | tctgtgctgtgagtaggccgaattcaggatacagcaccctcacctttgggaaggggactatg |
| | cttctagtctctccagatatccagaaccctgaccctgccgtgtaccagctgagagactctaa |
| | atccagtgacaagtctgtctgcctattcaccgattttgattctcaaacaaatgtgtcacaaa |
| | gtaaggattctgatgtgtatatcacagacaaaactgtgctagacatgaggtctatggacttc |
| | aagagcaacagtgctgtggcctggagcaacaaatctgactttgcatgtgcaaacgccttcaa |
| | caacagcattattccagaagacaccttcttccccagcccagaaagttcctgtgatgtcaagc |
| | tggtcgagaaaagctttgaaacagatacgaacctaaactttcaaaacctgtcagtgattggg |
| | ttccgaatcctcctcctgaaagtggccgggtttaatctgctcatgacgctgcggctgtggtc |
| | cagctga (SEQ ID NO: 68) |
| β chain | atgcttagtcctgacctgcctgactctgcctggaacaccaggctcctctgccatgtcatgct |
| with | ttgtctcctgggagcagtttcagtggctgctggagtcatccagtccccaagacatctgatca |
| TRBC1 | aagaaaagagggaaacagccactctgaaatgctatcctatccctagacacgacactgtctac |
| constant | tggtaccagcagggtccaggtcaggacccccagttcctcatttcgttttatgaaaagatgca |
| domain | gagcgataaaggaagcatccctgatcgattctcagctcaacagttcagtgactatcattctg |
| | aactgaacatgagctccttggagctgggggactcagccctgtacttctgtgccagcagtgtc |
| | aggactccgtcggggcaagagacccagtacttcgggccaggcacgcggctcctggtgctcga |
| | ggacctgaacaaggtgttcccacccgaggtcgctgtgtttgagccatcagaagcagagatct |
| | cccacacccaaaaggccacactggtgtgcctggccacaggcttcttccccgaccacgtggag |
| | ctgagctggtgggtgaatgggaaggaggtgcacagtggggtcagcacggacccgcagcccct |
| | caaggagcagcccgccctcaatgactccagatactgcctgagcagccgcctgagggtctcgg |
| | ccaccttctggcagaacccccgcaaccacttccgctgtcaagtccagttctacgggctctcg |
| | gagaatgacgagtggacccaggatagggccaaacccgtcacccagatcgtcagcgccgaggc |
| | ctggggtagagcagactgtggctttacctcggtgtcctaccagcaaggggtcctgtctgcca |
| | ccatcctctatgagatcctgctagggaaggccaccctgtatgctgtgctggtcagcgccctt |
| | gtgttgatggccatggtcaagagaaaggatttctga (SEQ ID NO: 69) |
| β chain | atgcttagtcctgacctgcctgactctgcctggaacaccaggctcctctgccatgtcatgct |
| with | ttgtctcctgggagcagtttcagtggctgctggagtcatccagtccccaagacatctgatca |
| TRBC2 | aagaaaagagggaaacagccactctgaaatgctatcctatccctagacacgacactgtctac |
| constant | tggtaccagcagggtccaggtcaggacccccagttcctcatttcgttttatgaaaagatgca |
| domain | gagcgataaaggaagcatccctgatcgattctcagctcaacagttcagtgactatcattctg |
| | aactgaacatgagctccttggagctgggggactcagccctgtacttctgtgccagcagtgtc |
| | aggactccgtcggggcaagagacccagtacttcgggccaggcacgcggctcctggtgctcga |
| | ggacctgaaaaacgtgttcccacccgaggtcgctgtgtttgagccatcagaagcagagatct |
| | cccacacccaaaaggccacactggtgtgcctggccacaggcttctaccccgaccacgtggag |
| | ctgagctggtgggtgaatgggaaggaggtgcacagtggggtcagcacagacccgcagcccct |
| | caaggagcagcccgccctcaatgactccagatactgcctgagcagccgcctgagggtctcgg |
| | ccaccttctggcagaacccccgcaaccacttccgctgtcaagtccagttctacgggctctcg |
| | gagaatgacgagtggacccaggatagggccaaacctgtcacccagatcgtcagcgccgaggc |
| | ctggggtagagcagactgtggcttcacctccgagtcttaccagcaaggggtcctgtctgcca |
| | ccatcctctatgagatcttgctagggaaggccaccttgtatgccgtgctggtcagtgccctc |
| | gtgctgatggccatggtcaagagaaaggattccagaggctaa (SEQ ID NO: 70) |
| α chain | atgtggggagtgtttctgctgtacgtgtccatgaagatgggcggcaccaccggccagaacat |
| with | cgatcagcctacagagatgaccgccaccgagggcgccatcgtgcagatcaattgcacctacc |
| TRAC | agaccagcggcttcaacggcctgttctggtatcagcagcatgccggcgaggcccctaccttc |
| constant | ctgagctacaatgtgctggacggcctggaagagaagggcagattcagcagcttcctgtccag |
| domain | aagcaagggctacagctacctgctgctgaaagaactgcagatgaaggacagcgcctcctacc |
| | tgtgcgccgtgtccagacctaatagcggctacagcaccctgaccttcggcaagggaacaatg |
| | ctgctggtgtcccctgacattcagaaccccgatcctgccgtgtaccagctgagagacagcaa |
| | gagcagcgacaagagcgtgtgcctgttcaccgacttcgacagccagaccaacgtgtcccaga |
| | gcaaggactccgacgtgtacatcaccgataagtgcgtgctggacatgcggagcatggacttc |
| | aagagcaacagcgccgtggcctggtccaacaagagcgatttcgcctgcgccaacgccttcaa |
| | caacagcattatccccgaggacacattcttcccaagtcctgagagcagctgcgacgtgaagc |
| | tggtggaaaagagcttcgagacagacaccaacctgaacttccagaacctgagcgtgatcggc |
| | ttccggatcctgctgcttaaggtggccggcttcaatctgctgatgaccctgagactgtggtc |
| | cagctga (SEQ ID NO: 71) |
| β chain | atgctttctcctgatcttcctgacagcgcctggaacaccagactgctgtgtcacgtgatgct |
| with | gtgtctgctgggagccgtgtctgttgccgctggcgttatccagtctcctcggcacctgatca |
| TRBC2 | aagagaagagagagacagccacactgaagtgctaccccattccacggcacgacaccgtgtac |
| constant | tggtatcagcaaggcccaggccaggatcctcagttcctgatcagcttctacgagaagatgca |
| domain | gagcgacaagggcagcatccccgacagattttctgcccagcagttcagcgactaccacagcg |
| | agctgaacatgagcagcctggaactgggcgatagcgccctgtacttttgtgccagctctgtg |
| | cggacacccagcggccaagaaacccagtattttggccccggaacacggctgctggtgctgga |
| | agatctgaagaacgtgttcccacctgaggtggccgtgttcgagccttctgaggccgagatct |
| | ctcacacccagaaagccacactcgtgtgtctggccaccggcttctatcccgatcacgtggaa |
| | ctgtcttggtgggtcaacggcaaagaggtgcacagcggcgtctgtaccgatcctcagcctct |
| | gaaagaacagcccgctctgaacgacagccggtactgtctgagcagcagactgagagtgtccg |
| | ccaccttctggcagaaccccagaaaccacttcagatgccaggtgcagttctacggcctgagc |
| | gagaacgatgagtggacccaggatagagccaagcctgtgacacagatcgtgtctgccgaagc |
| | ctggggcagagccgattgtggctttaccagcgagagctaccagcagggcgtgctgtctgcca |
| | caatcctgtacgagatcctgctgggaaaagccactctgtacgccgtgctggtgtctgccctg |
| | gtgctgatggccatggtcaagcggaaggatagcaggggctaa (SEQ ID NO: 72) |

hTERT #3

| Region | Example nucleotide sequence |
|---|---|
| α chain | atggctttgcagagcactctgggggcggtgtggctagggcttctcctcaactctctctggaa |
| with | ggttgcagaaagcaaggaccaagtgtttcagccttccacagtggcatcttcagagggagctg |
| TRAC | tggtggaaatcttctgtaatcactctgtgtccaatgcttacaacttcttctggtaccttcac |
| constant | ttcccgggatgtgcaccaagactccttgttaaaggctcaaagccttctcagcagggacgata |
| domain | caacatgacctatgaacggttctcttcatcgctgctcatcctccaggtgcgggaggcagatg |
| | ctgctgtttactactgtgctgtggagactgggggtggtgctacaaacaagctcatctttgga |
| | actggcactctgcttgctgtccagccaaatatccagaaccctgaccctgccgtgtaccagct |
| | gagagactctaaatccagtgacaagtctgtctgcctattcaccgattttgattctcaaacaa |
| | atgtgtcacaaagtaaggattctgatgtgtatatcacagacaaaactgtgctagacatgagg |
| | tctatggacttcaagagcaacagtgctgtggcctggagcaacaaatctgactttgcatgtgc |
| | aaacgccttcaacaacagcattattccagaagacaccttcttccccagcccagaaagttcct |
| | gtgatgtcaagctggtcgagaaaagctttgaaacagatacgaacctaaactttcaaaacctg |
| | tcagtgattgggttccgaatcctcctcctgaaagtggccgggtttaatctgctcatgacgct |
| | gcggctgtggtccagctga (SEQ ID NO: 73) |
| β chain | atgagcatcgggctcctgtgctgtgtggccttttctctcctgtgggcaagtccagtgaatgc |
| with | tggtgtcactcagaccccaaaattccaggtcctgaagacaggacagagcatgacactgcagt |
| TRBC1 | gtgcccaggatatgaaccataactccatgtactggtatcgacaagacccaggcatgggactg |
| constant | aggctgatttattactcagcttctgagggtaccactgacaaaggagaagtccccaatggcta |
| domain | caatgtctccagattaaacaaacgggagttctcgctcaggctggagtcggctgctccctccc |
| | agacatctgtgtacttctgtgccagcagtgaattttggcttacccaagagacccagtacttc |
| | gggccaggcacgcggctcctggtgctcgaggacctgaacaaggtgttcccacccgaggtcgc |
| | tgtgtttgagccatcagaagcagagatctcccacacccaaaaggccacactggtgtgcctgg |
| | ccacaggcttcttccccgaccacgtggagctgagctggtgggtgaatgggaaggaggtgcac |
| | agtggggtcagcacggacccgcagcccctcaaggagcagcccgccctcaatgactccagata |
| | ctgcctgagcagccgcctgagggtctcggccaccttctggcagaacccccgcaaccacttcc |
| | gctgtcaagtccagttctacgggctctcggagaatgacgagtggacccaggatagggccaaa |
| | cccgtcacccagatcgtcagcgccgaggcctggggtagagcagactgtggctttacctcggt |
| | gtcctaccagcaaggggtcctgtctgccaccatcctctatgagatcctgctagggaaggcca |
| | ccctgtatgctgtgctggtcagcgcccttgtgttgatggccatggtcaagagaaaggatttc |
| | tga (SEQ ID NO: 74) |
| β chain | atgagcatcgggctcctgtgctgtgtggccttttctctcctgtgggcaagtccagtgaatgc |
| with | tggtgtcactcagaccccaaaattccaggtcctgaagacaggacagagcatgacactgcagt |
| TRBC2 | gtgcccaggatatgaaccataactccatgtactggtatcgacaagacccaggcatgggactg |
| constant | aggctgatttattactcagcttctgagggtaccactgacaaaggagaagtccccaatggcta |
| domain | caatgtctccagattaaacaaacgggagttctcgctcaggctggagtcggctgctccctccc |
| | agacatctgtgtacttctgtgccagcagtgaattttggcttacccaagagacccagtacttc |
| | gggccaggcacgcggctcctggtgctcgaggacctgaaaaacgtgttcccacccgaggtcgc |
| | tgtgtttgagccatcagaagcagagatctcccacacccaaaaggccacactggtgtgcctgg |
| | ccacaggcttctaccccgaccacgtggagctgagctggtgggtgaatgggaaggaggtgcac |
| | agtggggtcagcacagacccgcagcccctcaaggagcagcccgccctcaatgactccagata |
| | ctgcctgagcagccgcctgagggtctcggccaccttctggcagaacccccgcaaccacttcc |
| | gctgtcaagtccagttctacgggctctcggagaatgacgagtggacccaggatagggccaaa |
| | cctgtcacccagatcgtcagcgccgaggcctggggtagagcagactgtggcttcacctccga |
| | gtcttaccagcaaggggtcctgtctgccaccatcctctatgagatcttgctagggaaggcca |
| | ccttgtatgccgtgctggtcagtgccctcgtgctgatggccatggtcaagagaaaggattcc |
| | agaggctaa (SEQ ID NO: 75) |
| α chain | atggctctgcagtctacactgggcgctgtttggctgggcctgctgctgaattctctgtggaa |
| with | ggtggccgagagcaaggaccaggtgttccagccttctacagtggcctcttctgagggcgccg |
| TRAC | tggtggaaatcttctgcaaccacagcgtgtccaacgcctacaacttcttctggtatctgcac |
| constant | ttccccggctgcgcccctagactgcttgtgaagggaagcaagcctagccagcagggcagata |
| domain | caacatgacctacgagcggttcagcagctccctgctgattctgcaagtgcgggaagccgatg |
| | ccgccgtgtattactgtgctgtggaaacaggcggcggagccaccaacaagctgatctttgga |
| | accggcacactgctggccgtgcagcccaatattcagaaccctgatcctgccgtgtaccagct |
| | gagagacagcaagagcagcgacaagagcgtgtgcctgttcaccgacttcgacagccagacca |
| | acgtgtcccagtccaaggacagcgacgtgtacatcaccgataagtgcgtgctggacatgcgg |
| | agcatggacttcaagagcaacagcgccgtggcctggtccaacaagagcgatttcgcctgcgc |
| | caatgccttcaacaacagcattatccccgaggacacattcttcccaagtcctgagagcagct |
| | gcgacgtgaagctggtggaaaagagcttcgagacagacaccaacctgaacttccagaacctg |
| | agcgtgatcggcttcaggatcctcctgctgaaagtggccggcttcaacctgctgatgaccct |
| | gagactgtggtccagctga (SEQ ID NO: 76) |
| β chain | atgtctattggactgctgtgctgcgtggccttcagcctgctttgggcctctcctgtgaatgc |
| with | cggcgtgacccagacacctaagttccaggtgctgaaaaccggccagagcatgaccctgcagt |
| TRBC2 | gcgcccaggacatgaaccacaacagcatgtactggtacagacaggaccccggcatgggcctg |
| constant | agactgatctactactctgccagcgagggcaccaccgacaaaggcgaagtgcccaatggcta |
| domain | caacgtgtcccggctgaacaagagagagttcagcctgaggctggaaagcgccgctccatctc |
| | agaccagcgtgtacttttgtgccagcagcgagttctggctgacccaagagacacagtacttc |
| | ggccctggcaccagactgctggtcctggaagatctgaagaacgtgttcccacctgaggtggc |
| | cgtgttcgagccttctgaggccgagatcagccacacacagaaagccacactcgtgtgtctgg |
| | ccaccggcttctatcccgatcacgtggaactgtcttggtgggtcaacggcaaagaggtgcac |
| | agcggcgtctgtaccgatcctcagcctctgaaagagcagcccgctctgaacgacagcagata |
| | ctgcctgagcagcagactgagagtgtccgccaccttctggcagaaccccagaaaccacttca |
| | gatgccaggtgcagttctacggcctgagcgagaacgatgagtggacccaggatagagccaag |
| | cctgtgacacagatcgtgtctgccgaagcctggggcagagccgattgtggctttaccagcga |
| | gagctaccagcagggcgtgctgtctgccacaatcctgtacgagatcctgctgggcaaagcca |
| | ctctgtacgccgtgctggtgtctgccctggtgctgatggccatggtcaagcggaaggatagc |
| | aggggctaa (SEQ ID NO: 77) |

hTERT #4

| Region | Example nucleotide sequence |
|---|---|
| α chain | atgaagaggatattgggagctctgctggggctcttgagtgcccaggtttgctgtgtgagagg |
| with | aatacaagtggagcagagtcctccagacctgattctccaggagggagccaattccacgctgc |
| TRAC | ggtgcaatttttctgactctgtgaacaatttgcagtggtttcatcaaaacccttggggacag |
| constant | ctcatcaacctgttttacattccctcagggacaaaacagaatggaagattaagcgccacgac |
| domain | tgtcgctacggaacgctacagcttattgtacatttcctcttcccagaccacagactcaggcg |
| | tttatttctgtgctgtgtcccacggaaggggtggtgctacaaacaagctcatctttggaact |
| | ggcactctgcttgctgtccagccaaatatccagaaccctgaccctgccgtgtaccagctgag |
| | agactctaaatccagtgacaagtctgtctgcctattcaccgattttgattctcaaacaaatg |
| | tgtcacaaagtaaggattctgatgtgtatatcacagacaaaactgtgctagacatgaggtct |
| | atggacttcaagagcaacagtgctgtggcctggagcaacaaatctgactttgcatgtgcaaa |
| | cgccttcaacaacagcattattccagaagacaccttcttccccagcccagaaagttcctgtg |
| | atgtcaagctggtcgagaaaagctttgaaacagatacgaacctaaactttcaaaacctgtca |
| | gtgattgggttccgaatcctcctcctgaaagtggccgggtttaatctgctcatgacgctgcg |
| | gctgtggtccagctga (SEQ ID NO: 78) |
| β chain | atgagcatcgggctcctgtgctgtgtggccttttctctcctgtgggcaagtccagtgaatgc |
| with | tggtgtcactcagaccccaaaattccaggtcctgaagacaggacagagcatgacactgcagt |
| TRBC1 | gtgcccaggatatgaaccataactccatgtactggtatcgacaagacccaggcatgggactg |
| constant | aggctgatttattactcagcttctgagggtaccactgacaaaggagaagtccccaatggcta |
| domain | caatgtctccagattaaacaaacgggagttctcgctcaggctggagtcggctgctccctccc |
| | agacatctgtgtacttctgtgccagcgacagggtattagggtacgagcagtacttcgggccg |
| | ggcaccaggctcacggtcacagaggacctgaacaaggtgttcccacccgaggtcgctgtgtt |
| | tgagccatcagaagcagagatctcccacacccaaaaggccacactggtgtgcctggccacag |
| | gcttcttccccgaccacgtggagctgagctggtgggtgaatgggaaggaggtgcacagtggg |
| | gtcagcacggacccgcagcccctcaaggagcagcccgccctcaatgactccagatactgcct |
| | gagcagccgcctgagggtctcggccaccttctggcagaacccccgcaaccacttccgctgtc |
| | aagtccagttctacgggctctcggagaatgacgagtggacccaggatagggccaaacccgtc |
| | acccagatcgtcagcgccgaggcctggggtagagcagactgtggctttacctcggtgtccta |
| | ccagcaaggggtcctgtctgccaccatcctctatgagatcctgctagggaaggccaccctgt |
| | atgctgtgctggtcagcgcccttgtgttgatggccatggtcaagagaaaggatttctga |
| | (SEQ ID NO: 79) |
| β chain | atgagcatcgggctcctgtgctgtgtggccttttctctcctgtgggcaagtccagtgaatgc |
| with | tggtgtcactcagaccccaaaattccaggtcctgaagacaggacagagcatgacactgcagt |
| TRBC2 | gtgcccaggatatgaaccataactccatgtactggtatcgacaagacccaggcatgggactg |
| constant | aggctgatttattactcagcttctgagggtaccactgacaaaggagaagtccccaatggcta |
| domain | caatgtctccagattaaacaaacgggagttctcgctcaggctggagtcggctgctccctccc |
| | agacatctgtgtacttctgtgccagcgacagggtattagggtacgagcagtacttcgggccg |
| | ggcaccaggctcacggtcacagaggacctgaaaaacgtgttcccacccgaggtcgctgtgtt |
| | tgagccatcagaagcagagatctcccacacccaaaaggccacactggtgtgcctggccacag |
| | gcttctaccccgaccacgtggagctgagctggtgggtgaatgggaaggaggtgcacagtggg |
| | gtcagcacagacccgcagcccctcaaggagcagcccgccctcaatgactccagatactgcct |
| | gagcagccgcctgagggtctcggccaccttctggcagaacccccgcaaccacttccgctgtc |
| | aagtccagttctacgggctctcggagaatgacgagtggacccaggatagggccaaacctgtc |
| | acccagatcgtcagcgccgaggcctggggtagagcagactgtggcttcacctccgagtctta |
| | ccagcaaggggtcctgtctgccaccatcctctatgagatcttgctagggaaggccaccttgt |
| | atgccgtgctggtcagtgccctcgtgctgatggccatggtcaagagaaaggattccagaggc |
| | taa (SEQ ID NO: 80) |
| α chain | atgaagagaatcctgggcgccctgctgggactgctgtctgcccaagtctgttgtgtgcgggg |
| with | catccaggtggaacagagccctcctgatctgatcctgcaagagggcgccaacagcaccctga |
| TRAC | gatgcaacttcagcgacagcgtgaacaacctgcagtggttccaccagaatccttggggccag |
| constant | ctgatcaacctgttctacatccccagcggcaccaagcagaacggcagactgtctgctaccac |
| domain | cgtggccaccgagagatacagcctgctgtacatcagcagcagccagaccacagacagcggcg |
| | tgtacttctgtgccgtgtctcatggaagaggcggagccaccaacaagctgatctttggcaca |
| | ggcacactgctggccgtgcagcccaatattcagaaccctgatccagccgtgtaccagctgag |
| | agacagcaagagcagcgacaagagcgtgtgtctgttcaccgacttcgactcccagaccaacg |
| | tgtcccagagcaaggactccgacgtgtacatcaccgataagtgcgtgctggacatgcggagc |
| | atggacttcaagagcaacagcgccgtggcctggtccaacaagagcgattttgcctgcgccaa |
| | cgccttcaacaacagcattatccccgaggacacattcttcccaagtcctgagagcagctgcg |
| | acgtgaagctggtggaaaagagcttcgagacagacaccaacctgaacttccagaacctgagc |
| | gtgatcggcttccggatcctgctgcttaaggtggccggcttcaacctgctgatgaccctgag |
| | actgtggtccagctga (SEQ ID NO: 81) |
| β chain | atgtctattggactgctgtgctgcgtggccttcagcctgctttgggcctctcctgtgaatgc |
| with | cggcgtgacccagacacctaagttccaggtgctgaaaaccggccagagcatgaccctgcagt |
| TRBC2 | gcgcccaggacatgaaccacaacagcatgtactggtacagacaggaccccggcatgggcctg |
| constant | agactgatctactactctgccagcgagggcaccaccgacaaaggcgaagtgcccaatggcta |
| domain | caacgtgtcccggctgaacaagagagagttcagcctgaggctggaaagcgccgctccatctc |
| | agaccagcgtgtacttctgcgccagcgatagagtgctgggctacgagcagtatttcggccct |
| | ggcaccagactgaccgtgaccgaggatctgaagaacgtgttcccacctgaggtggccgtgtt |
| | cgagccttctgaggccgagatcagccacacacagaaagccacactcgtgtgtctggccaccg |
| | gcttctatcccgatcacgtggaactgtcttggtgggtcaacggcaaagaggtgcacagcggc |
| | gtctgtaccgatcctcagcctctgaaagagcagcccgctctgaacgacagcagatactgcct |
| | gagcagcagactgagagtgtccgccaccttctggcagaaccccagaaaccacttcagatgcc |
| | aggtgcagttctacggcctgagcgagaacgatgagtggacccaggatagagccaagcctgtg |
| | acacagatcgtgtctgccgaagcctggggcagagccgattgtggctttaccagcgagagcta |
| | ccagcagggcgtgctgtctgccacaatcctgtacgagatcctgctgggcaaagccactctgt |
| | acgccgtgctggtgtctgccctggtgctgatggccatggtcaagcggaaggatagcaggggc |
| | taa (SEQ ID NO: 82) |

In one aspect, the present invention provides a polynucleotide comprising or consisting of one or more nucleotide sequences selected from the group consisting of SEQ ID NOs: 63 to 82, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding an α chain, comprising of consisting of SEQ ID NO: 63 or 66, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding a β chain, comprising of consisting of SEQ ID NO: 64, 65, or 67, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding an α chain, comprising of consisting of SEQ ID NO: 68 or 71, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding a β chain, comprising of consisting of SEQ ID NO: 69, 70, or 72, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding an α chain, comprising of consisting of SEQ ID NO: 73 or 76, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding a β chain, comprising of consisting of SEQ ID NO: 74, 75, or 77, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding an α chain, comprising of consisting of SEQ ID NO: 78 or 81, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding a β chain, comprising of consisting of SEQ ID NO: 79, 80, or 82, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising an α chain encoded by the nucleotide sequence of SEQ ID NO: 63 or 66, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising a β chain encoded by the nucleotide sequence of SEQ ID NO: 64, 65, or 67, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising an α chain encoded by the nucleotide sequence of SEQ ID NO: 68 or 71, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising a β chain encoded by the nucleotide sequence of SEQ ID NO: 69, 70, or 72, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising an α chain encoded by the nucleotide sequence of SEQ ID NO: 73 or 76, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising a β chain encoded by the nucleotide sequence of SEQ ID NO: 74, 75, or 77, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising an α chain encoded by the nucleotide sequence of SEQ ID NO: 78 or 81, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising a β chain encoded by the nucleotide sequence of SEQ ID NO: 79, 80, or 82, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

Further provided by the invention are polynucleotide sequences derived from the sequences present in the table above. For example, the present invention provides a polynucleotide encoding a variable region of a TCR according to the invention, wherein the polynucleotide comprises a stretch of nucleotides of any one of SEQ ID NOs: 63 to 82.

The variant sequences may have additions, deletions or substitutions, of one or more bases. If the variation involves addition(s) or deletion(s) they may either occur in threes or be balanced (i.e. an addition for each deletion) so that the variation does not cause a frameshift for translation of the remainder of the sequence. Some or all of the variations may be “silent” in the sense that they do not affect the sequence of the encoded protein due to the degeneracy of the genetic code.

Some or all of the variations may produce conservative amino acid substitutions, additions or deletions as explained above. The variation may be concentrated in one or more regions, such as the regions encoding the constant regions, the linker, or the framework regions of the α or β chains, or they may be spread throughout the molecule. The variant sequence should retain the capacity to encode all or part of a TCR amino acid sequence which binds to a hTERT peptide.

### Survivin-Specific TCR Sequences

Examples of Survivin-specific TCR nucleotide sequences are provided in the tables below.

The present invention also encompasses the TCR amino acid sequences encoded by the TCR nucleotide sequences provided in the tables below, and variants thereof.

| Region | Example nucleotide sequence |
|---|---|
| α chain | atggagtcattcctgggaggtgttttgctgattttgtggcttcaagtggactgggtgaagag |
| with | ccaaaagatagaacagaattccgaggccctgaacattcaggagggtaaaacggccaccctga |
| TRAC | cctgcaactatacaaactattccccagcatacttacagtggtaccgacaagatccaggaaga |
| constant | ggccctgttttcttgctactcatacgtgaaaatgagaaagaaaaaaggaaagaaagactgaa |
| domain | ggtcacctttgataccacccttaaacagagtttgtttcatatcacagcctcccagcctgcag |
| | actcagctacctacctctgtgctctagaccggatggatagcagctataaattgatcttcggg |
| | agtgggaccagactgctggtcaggcctgatatccagaaccctgaccctgccgtgtaccagct |
| | gagagactctaaatccagtgacaagtctgtctgcctattcaccgattttgattctcaaacaa |
| | atgtgtcacaaagtaaggattctgatgtgtatatcacagacaaaactgtgctagacatgagg |
| | tctatggacttcaagagcaacagtgctgtggcctggagcaacaaatctgactttgcatgtgc |
| | aaacgccttcaacaacagcattattccagaagacaccttcttccccagcccagaaagttcct |
| | gtgatgtcaagctggtcgagaaaagctttgaaacagatacgaacctaaactttcaaaacctg |
| | tcagtgattgggttccgaatcctcctcctgaaagtggccgggtttaatctgctcatgacgct |
| | gcggctgtggtccagctga (SEQ ID NO: 83) |
| β chain | atgagcatcggcctcctgtgctgtgcagccttgtctctcctgtgggcaggtccagtgaatgc |
| with | tggtgtcactcagaccccaaaattccaggtcctgaagacaggacagagcatgacactgcagt |
| TRBC1 | gtgcccaggatatgaaccatgaatacatgtcctggtatcgacaagacccaggcatggggctg |
| constant | aggctgattcattactcagttggtgctggtatcactgaccaaggagaagtccccaatggcta |
| domain | caatgtctccagatcaaccacagaggatttcccgctcaggctgctgtcggctgctccctccc |
| | agacatctgtgtacttctgtgccagcagttacgatcaggacggtgaagctttctttggacaa |
| | ggcaccagactcacagttgtagaggacctgaacaaggtgttcccacccgaggtcgctgtgtt |
| | tgagccatcagaagcagagatctcccacacccaaaaggccacactggtgtgcctggccacag |
| | gcttcttccccgaccacgtggagctgagctggtgggtgaatgggaaggaggtgcacagtggg |
| | gtcagcacggacccgcagcccctcaaggagcagcccgccctcaatgactccagatactgcct |
| | gagcagccgcctgagggtctcggccaccttctggcagaacccccgcaaccacttccgctgtc |
| | aagtccagttctacgggctctcggagaatgacgagtggacccaggatagggccaaacccgtc |
| | acccagatcgtcagcgccgaggcctggggtagagcagactgtggctttacctcggtgtccta |
| | ccagcaaggggtcctgtctgccaccatcctctatgagatcctgctagggaaggccaccctgt |
| | atgctgtgctggtcagcgcccttgtgttgatggccatggtcaagagaaaggatttctga |
| | (SEQ ID NO: 84) |
| β chain | atgagcatcggcctcctgtgctgtgcagccttgtctctcctgtgggcaggtccagtgaatgc |
| with | tggtgtcactcagaccccaaaattccaggtcctgaagacaggacagagcatgacactgcagt |
| TRBC2 | gtgcccaggatatgaaccatgaatacatgtcctggtatcgacaagacccaggcatggggctg |
| constant | aggctgattcattactcagttggtgctggtatcactgaccaaggagaagtccccaatggcta |
| domain | caatgtctccagatcaaccacagaggatttcccgctcaggctgctgtcggctgctccctccc |
| | agacatctgtgtacttctgtgccagcagttacgatcaggacggtgaagctttctttggacaa |
| | ggcaccagactcacagttgtagaggacctgaaaaacgtgttcccacccgaggtcgctgtgtt |
| | tgagccatcagaagcagagatctcccacacccaaaaggccacactggtgtgcctggccacag |
| | gcttctaccccgaccacgtggagctgagctggtgggtgaatgggaaggaggtgcacagtggg |
| | gtcagcacagacccgcagcccctcaaggagcagcccgccctcaatgactccagatactgcct |
| | gagcagccgcctgagggtctcggccaccttctggcagaacccccgcaaccacttccgctgtc |
| | aagtccagttctacgggctctcggagaatgacgagtggacccaggatagggccaaacctgtc |
| | acccagatcgtcagcgccgaggcctggggtagagcagactgtggcttcacctccgagtctta |
| | ccagcaaggggtcctgtctgccaccatcctctatgagatcttgctagggaaggccaccttgt |
| | atgccgtgctggtcagtgccctcgtgctgatggccatggtcaagagaaaggattccagaggc |
| | taa (SEQ ID NO: 85) |

| Region | Example nucleotide sequence |
|---|---|
| α chain | atggaatcctttcttggcggcgtgctgctgatcctgtggctgcaagtggattgggtcaagag |
| with | ccagaagatcgagcagaacagcgaggccctgaacatccaagagggcaagaccgccacactga |
| TRAC | cctgcaactacaccaactacagccccgcctacctgcagtggtacagacaggatccaggcaga |
| constant | ggccctgtgttcctgctgctcatccgcgagaacgagaaagagaagcgcaaagaacggctgaa |
| domain | agtgaccttcgacaccacactgaagcagagcctgttccacatcaccgcctctcagcctgccg |
| | atagcgccacatatctgtgcgccctggacagaatggacagcagctacaagctgatcttcggc |
| | agcggcaccagactgctcgtgcggcccgatattcagaaccctgatcctgccgtgtaccagct |
| | gagagacagcaagagcagcgacaagagcgtgtgcctgttcaccgacttcgacagccagacca |
| | acgtgtcccagagcaaggacagcgacgtgtacatcaccgataagtgcgtgctggacatgcgg |
| | agcatggacttcaagagcaacagcgccgtggcctggtccaacaagagcgatttcgcctgcgc |
| | caacgccttcaacaacagcattatccccgaggacacattcttcccaagtcctgagagcagct |
| | gcgacgtgaagctggtggaaaagagcttcgagacagacaccaacctgaacttccagaacctg |
| | agcgtgatcggcttccggattctgctgctgaaggtggccggcttcaacctgctgatgaccct |
| | gagactgtggtccagctga (SEQ ID NO: 86) |
| β chain | atgtctattggactgctttgttgcgccgctctgagcctgctttgggccggacctgttaatgc |
| with | tggcgtgacccagacacctaagttccaggtgctgaaaaccggccagagcatgaccctgcagt |
| TRBC2 | gcgcccaggatatgaaccacgagtacatgagctggtacagacaggaccctggcatgggcctg |
| constant | agactgatccactattctgtcggagccggcatcaccgaccagggcgaagttcctaatggcta |
| domain | caacgtgtccagaagcaccaccgaggacttcccactgagactgctgtctgccgctcctagcc |
| | agaccagcgtgtacttttgtgccagcagctacgaccaggacggcgaggccttttttggccaa |
| | ggcaccagactgaccgtggtggaagatctgaagaacgtgttcccacctgaggtggccgtgtt |
| | cgagccttctgaggccgagatcagccacacacagaaagccacactcgtgtgtctggccaccg |
| | gcttctatcccgatcacgtggaactgtcttggtgggtcaacggcaaagaggtgcacagcggc |
| | gtctgtaccgatcctcagcctctgaaagagcagcccgctctgaacgacagcagatactgcct |
| | gagcagcagactgagagtgtccgccaccttctggcagaaccccagaaaccacttcagatgcc |
| | aggtgcagttctacggcctgagcgagaacgatgagtggacccaggatagagccaagcctgtg |
| | acacagatcgtgtctgccgaagcctggggcagagccgattgtggctttaccagcgagagcta |
| | ccagcagggcgttctgtctgccaccatcctgtacgagatcctgctgggcaaagccactctgt |
| | acgccgtgctggtgtctgccctggtgctgatggccatggtcaagcggaaggatagcaggggc |
| | taa (SEQ ID NO: 87) |

In one aspect, the present invention provides a polynucleotide comprising or consisting of one or more nucleotide sequences selected from the group consisting of SEQ ID NOs: 83 to 87, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding an α chain, comprising of consisting of SEQ ID NO: 83, or a variant thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding a β chain, comprising of consisting of SEQ ID NO: 84, or a variant thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding a β chain, comprising of consisting of SEQ ID NO: 85, or a variant thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding an α chain, comprising of consisting of SEQ ID NO: 86, or a variant thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a polynucleotide encoding a β chain, comprising of consisting of SEQ ID NO: 87, or a variant thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising an α chain encoded by the nucleotide sequence of SEQ ID NO: 83, or a variant thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising a β chain encoded by the nucleotide sequence of SEQ ID NO: 84, or a variant thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising a β chain encoded by the nucleotide sequence of SEQ ID NO: 85, or a variant thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising an α chain encoded by the nucleotide sequence of SEQ ID NO: 86, or a variant thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one aspect, the present invention provides a TCR comprising a β chain encoded by the nucleotide sequence of SEQ ID NO: 87, or a variant thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

Further provided by the invention are polynucleotide sequences derived from the sequences present in the table above. For example, the present invention provides a polynucleotide encoding a variable region of a TCR according to the invention, wherein the polynucleotide comprises a stretch of nucleotides of any one of SEQ ID NOs: 83 to 87.

The variant sequences may have additions, deletions or substitutions, of one or more bases. If the variation involves addition(s) or deletion(s) they may either occur in threes or be balanced (i.e. an addition for each deletion) so that the variation does not cause a frameshift for translation of the remainder of the sequence. Some or all of the variations may be “silent” in the sense that they do not affect the sequence of the encoded protein due to the degeneracy of the genetic code.

Some or all of the variations may produce conservative amino acid substitutions, additions or deletions as explained above. The variation may be concentrated in one or more regions, such as the regions encoding the constant regions, the linker, or the framework regions of the α or β chains, or they may be spread throughout the molecule. The variant sequence should retain the capacity to encode all or part of a TCR amino acid sequence which binds to a Survivin peptide.

### Codon Optimisation

The polynucleotides of the present invention may be codon-optimised.

Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation may also involve the removal of mRNA instability motifs and cryptic splice sites.

### Variants, Derivatives, Analogues, and Fragments

In addition to the specific polypeptides and polynucleotides mentioned herein, the invention also encompasses variants, derivatives, analogues, and fragments thereof.

In the context of the present invention, a “variant” of any given sequence is a sequence in which the specific sequence of residues (whether amino acid or nucleic acid residues) has been modified in such a manner that the polypeptide or polynucleotide in question substantially retains at least one of its functions. Suitably, for example, any amino acid changes in the TCR sequences should maintain the capacity of the TCR to bind the peptide presented by MHC molecules.

A variant amino acid or nucleic acid sequence can be obtained by addition, deletion, substitution, modification, replacement and/or variation of at least one residue present in the naturally-occurring polypeptide or polynucleotide. A variant amino acid sequence of the invention referred to as having up to three amino acid substitutions, additions or deletions may have, for example, one, two or three amino acid substitutions, additions or deletions. Suitably, for example, the variation may be concentrated in one or more regions, such as the constant regions, the linker, or the framework regions of the α or β chains, or they may be spread throughout the TCR.

The term “derivative” as used herein, in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its functions.

The term “analogue” as used herein, in relation to polypeptides or polynucleotides includes any mimetic, that is, a chemical compound that possesses at least one of the functions of the polypeptides or polynucleotides which it mimics.

Polypeptides of the present invention may have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues. A substitution may involve replacement of an amino acid for a similar amino acid (a conservative substitution). A similar amino acid is one which has a side chain moiety with related properties as grouped together, for example as shown below:
(i) basic side chains: lysine (K), arginine (R), histidine (H);
(ii) acidic side chains: aspartic acid (D) and glutamic acid (E);
(iii) uncharged polar side chains: asparagine (N), glutamine (Q), serine(S), threonine (T) and tyrosine (Y); or
(iv) non-polar side chains: glycine (G), alanine (A), valine (V), leucine (L), isoleucine (I), proline (P), phenylalanine (F), methionine (M), tryptophan (W) and cysteine (C).

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

The effect of additions, deletions, substitutions, modifications, replacements and/or variations may be predicted using any suitable prediction tool e.g. SIFT (Vaser, R., et al., 2016. Nature protocols, 11(1), pp. 1-9), PolyPhen-2 (Adzhubei, I., et al., 2013. Current protocols in human genetics, 76(1), pp. 7-20), CADD (Rentzsch, P., et al., 2021. Genome medicine, 13(1), pp. 1-12), REVEL (loannidis, N. M., et al., 2016. The American Journal of Human Genetics, 99(4), pp. 877-885), MetaLR (Dong, C., et al., 2015. Human molecular genetics, 24(8), pp. 2125-2137), and/or MutationAssessor (Reva, B., et al., 2011. Nucleic acids research, 39(17), pp. e118-e118).

Typically, a variant may have a certain identity with the subject amino acid sequence or the subject nucleotide sequence. Suitably, reference to a sequence which has a percent identity to any one of the SEQ ID NOs detailed herein refers to a sequence which has the stated percent identity over the entire length of the SEQ ID NO referred to.

In the present context, a variant amino acid sequence is taken to include an amino acid sequence which may be at least 50%, at least 55%, at least 60%, at least 65%, at least 70%, at least 75%, at least 80%, at least 85% or at least 90% identical, suitably at least 95%, at least 96%, at least 97%, at least 98% or at least 99% identical to the subject sequence. Although a variant can also be considered in terms of similarity (i.e. amino acid residues having similar chemical properties/functions), in the context of the present invention it is preferred to express in terms of sequence identity.

In the present context, a variant nucleotide sequence is taken to include a nucleotide sequence which may be at least 50%, at least 55%, at least 60%, at least 65%, at least 70%, at least 75%, at least 80%, at least 85% or at least 90% identical, suitably at least 95%, at least 96%, at least 97%, at least 98% or at least 99% identical to the subject sequence. Although a variant can also be considered in terms of similarity, in the context of the present invention it is preferred to express it in terms of sequence identity.

Sequence identity comparisons can be conducted by eye, or more usually, with the aid of readily available sequence comparison programs. These commercially available computer programs can calculate percent identity between two or more sequences.

Percent identity may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent identity therefore firstly requires the production of an optimal alignment, taking into consideration gap penalties. A suitable computer program for carrying out such an alignment is the GCG Wisconsin Bestfit package (see e.g. Devereux, J., et al., 1984. Nucleic acids research, 12(1), pp. 387-395). Examples of other software that can perform sequence comparisons include, but are not limited to, the BLAST package (see e.g. Altschul, S. F., et al., 1990. Journal of molecular biology, 215(3), pp. 403-410), BLAST 2 (see e.g. Tatusova, T. A. and Madden, T. L., 1999. FEMS microbiology letters, 174(2), pp. 247-250), FASTA (see e.g. Pearson, W. R. and Lipman, D. J., 1988. PNAS, 85(8), pp. 2444-2448.), EMBOSS Needle (Madeira, F., et al., 2019. Nucleic acids research, 47(W1), pp. W636-W641) and the GENEWORKS suite of comparison tools. For some applications, it is preferred to use EMBOSS Needle.

Although the final percent identity can be measured, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix.

Once the software has produced an optimal alignment, it is possible to calculate percent sequence identity. The software typically does this as part of the sequence comparison and generates a numerical result. The percent sequence identity may be calculated as the number of identical residues as a percentage of the total residues in the SEQ ID NO referred to.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants, derivatives, analogues and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded polypeptide. These methods are only illustrative of the numerous standard techniques known in the art for manipulation of DNA sequences and other known techniques may also be used.

### Vectors

In one aspect, the present invention provides a vector comprising a polynucleotide of the present invention.

A “vector” is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid, or facilitating the expression of the protein encoded by a segment of nucleic acid.

Examples of vectors used in recombinant nucleic acid techniques include, but are not limited to, plasmids, chromosomes, artificial chromosomes and viruses. The vector may be single stranded or double stranded. The vector may be linear or circular. The vector may also be, for example, a naked nucleic acid (e.g. DNA). In its simplest form, the vector may itself be a nucleotide of interest.

The term “vector” includes an expression vector i.e. a construct capable of in vivo or in vitro/ex vivo expression. Expression may be controlled by a vector sequence, or, for example in the case of insertion at a target site, expression may be controlled by a target sequence. A vector may be integrated or tethered to the cell's DNA.

The vectors used in the invention may be, for example, plasmid or viral vectors and may include a promoter for the expression of a polynucleotide and optionally a regulator of the promoter.

Viral vectors include but are not limited to adenovirus vector, an adeno-associated viral (AAV) vector, a herpes viral vector, a retroviral vector, a lentiviral vector, and a baculoviral vector.

In some embodiments, the vector is a retroviral vector. Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

There are many retroviruses, for example murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses. A detailed list of retroviruses may be found in Coffin et al (“Retroviruses” 1997 Cold Spring Harbour Laboratory Press Eds: J M Coffin, SM Hughes, HE Varmus pp 758-763). Lentiviruses also belong to the retrovirus family, but they can infect both dividing and non-dividing cells (Lewis et al (1992) EMBO J. 3053-3058).

The vector may be capable of transferring a nucleotide sequence encoding a TCR described herein to a cell, such as a T-cell, such that the cell expresses the TCR. Suitably, the vector will be capable of sustained high-level expression in T-cells, so that the introduced TCR may compete successfully with the endogenous TCR for a limited pool of CD3 molecules.

Increasing the supply of CD3 molecules may increase TCR expression, for example, in a cell that has been modified to express the TCRs of the invention. Accordingly, the vector of the invention may further comprise one or more genes encoding CD3-gamma, CD3-delta, CD3-epsilon and/or CD3-zeta. In some embodiments, the vector of the invention comprises a gene encoding CD3-zeta. The vector may comprise a gene encoding CD8. The vector may encode a selectable marker or a suicide gene, to increase the safety profile of the genetically engineered cell, e.g. a cell of the invention, or a cell that has been modified to express the TCRs of the invention (Bonini, C., et al., 1997. Science, 276(5319), pp. 1719-1724; Ciceri, F., et al., 2009. The lancet oncology, 10(5), pp. 489-500; Oliveira, G., et al., 2015. Science translational medicine, 7(317), p. 317ra198). The genes comprised in the vector of the invention may be linked by self-cleaving sequences, such as the 2A self-cleaving sequence.

Alternatively one or more separate vectors encoding a CD3 gene may be provided for co-transfer to a cell simultaneously, sequentially or separately with one or more vectors of the invention, e.g. one or more vectors encoding TCRs of the invention.

### Cells

In one aspect, the present invention provides a cell or a population of cells comprising a TCR, a polynucleotide or a vector according to the present invention.

The cell is not particularly limited and any suitable cell may be used. For example, the cell may be a T-cell, a lymphocyte, or a stem cell. The T-cell, the lymphocyte, or the stem cell may be selected from the group consisting of CD4+ cells, CD8+ cells, naive T-cells, memory stem T-cells, central memory T-cells, double negative T-cells, effector memory T-cells, effector T-cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, cytokine-induced killer (CIK) cells, hematopoietic stem cells and pluripotent stem cells.

The type of cell may be selected in order to provide desirable and advantageous in vivo persistence and to provide desirable and advantageous functions and characteristics to the cells of invention.

The cell may have been isolated from a subject.

The cell of the invention may be provided for use in adoptive cell transfer. As used herein, the term “adoptive cell transfer” may refer to the administration of a cell population to a patient. Typically, the cells are T-cells isolated from a subject and then genetically modified and cultured in vitro in order to express a TCR of the invention before being administered to the patient. Adoptive cell transfer may be allogenic or autologous.

By “autologous cell transfer” it is to be understood that the starting population of cells (which are then transduced according to a method of the invention, or are transduced with a vector according to the invention) is obtained from the same subject as that to which the transduced cell population is administered. Autologous transfer is advantageous as it avoids problems associated with immunological incompatibility and is available to subjects irrespective of the availability of a genetically matched donor.

By “allogeneic cell transfer” it is to be understood that the starting population of cells (which are then transduced according to a method of the invention, or are transduced with a vector according to the invention) is obtained from a different subject as that to which the transduced cell population is administered. Suitably, the donor will be genetically matched to the subject to which the cells are administered to minimise the risk of immunological incompatibility. Alternatively, the donor may be mismatched and unrelated to the patient.

Suitable doses of transduced cell populations are such as to be therapeutically and/or prophylactically effective. The dose to be administered may, for example, depend on the subject and condition to be treated, and may be readily determined by a skilled person.

In some embodiments, the cell is a T-cell. The cell may be derived from a T-cell isolated from a subject. The T-cell may be part of a mixed cell population isolated from the subject, such as a population of peripheral blood lymphocytes (PBL). T-cells within the PBL population may be activated by methods known in the art, such as using anti-CD3 and/or anti-CD28 antibodies or cell sized beads conjugated with anti-CD3 and/or anti-CD28 antibodies.

The T-cell may be a CD4⁺ helper T cell or a CD8⁺ cytotoxic T cell. The cell may be in a mixed population of CD4⁺ helper T cell/CD8⁺ cytotoxic T-cells. Polyclonal activation, for example using anti-CD3 antibodies optionally in combination with anti-CD28 antibodies will trigger the proliferation of CD4⁺ and CD8⁺ T-cells.

The cell may be isolated from the subject to which the genetically modified cell is to be adoptively transferred. In this respect, the cell may be made by isolating a T-cell from a subject, optionally activating the T-cell, transferring the TCR gene to the cell ex vivo. Subsequent immunotherapy of the subject may then be carried out by adoptive transfer of the TCR-transduced cells. As used herein this process refers to autologous T-cell transfer i.e. the TCR-transduced cells are administered to the same subject from which the T-cells were originally derived.

Alternatively the T-cell may be isolated from a different subject, such that it is allogeneic. The T-cell may be isolated from a donor subject. For example, if the subject is undergoing allogeneic hematopoietic stem cell transplantation (Allo-HSCT) or solid organ transplantation or cell transplantation or stem cell therapy, the cell may be derived from the donor, from which the organs, tissues or cells are derived. The donor and the subject undergoing treatment may be siblings.

Alternatively the cell may be, or may be derived from, a stem cell, such as a hematopoietic stem cell (HSC). Gene transfer into HSCs does not lead to TCR expression at the cell surface as stem cells do not express CD3 molecules. However, when stem cells differentiate into lymphoid precursors that migrate to the thymus, the initiation of CD3 expression leads to the surface expression of the introduced TCR in thymocytes.

An advantage of this approach is that the mature T-cells, once produced, express only the introduced TCR and little or no endogenous TCR chains, because the expression of the introduced TCR chains suppresses rearrangement of endogenous TCR gene segments to form functional TCR alpha and beta genes. A further benefit is that the gene-modified stem cells are a continuous source of mature T-cells with the desired antigen specificity. The cell may therefore be a gene-modified stem cell, preferably a gene-modified hematopoietic stem cell, which, upon differentiation, produces a T-cell expressing a TCR of the invention.

Other approaches known in the art may be used to reduce, limit, prevent, silence, or abrogate expression of endogenous genes in the cells of the invention or cells prepared by the methods of the invention.

As used herein the term “disrupting” refers to reducing, limiting, preventing, silencing, or abrogating expression of a gene. The person skilled in the art is able to use any method known in the art to disrupt an endogenous gene, e.g., any suitable method for genome editing, gene silencing, gene knock-down or gene knock-out.

For example, an endogenous gene may be disrupted with an artificial nuclease. An artificial nuclease is, e.g., an artificial restriction enzyme engineered to selectively target a specific polynucleotide sequence (e.g. encoding a gene of interest) and induce a double strand break in said polynucleotide sequence. Typically, the double strand break (DSB) will be repaired by error-prone non-homologous end joining (NHEJ) thereby resulting in the formation of a non-functional polynucleotide sequence, which may be unable to express an endogenous gene. In some embodiments, the artificial nuclease is selected from the group consisting of zinc finger nucleases (ZFN), transcription activator-like effector nucleases (TALEN) and CRISPR/Cas (e.g. CRISPR/Cas9).

The methods of preparing a cell (e.g. a T-cell) of the invention may comprise the step of targeted integration of an expression cassette into an endogenous gene (e.g. an endogenous TCR α chain gene and/or an endogenous TCR β chain gene). As used herein the term expression cassette refers to a polynucleotide sequence (e.g. a DNA polynucleotide sequence) comprising one or more polynucleotide sequences encoding one or more genes of interest such that said genes of interest are capable of expression. Endogenous sequences may facilitate expression from the expression cassette, and/or transcription control sequences within the expression cassette may facilitate expression. For example, the expression cassette may comprise a polynucleotide sequence of the invention, or a polynucleotide sequence encoding a TCR of the invention, operably linked to an expression control sequence, e.g. a promoter or an enhancer sequence. The one or more genes of interest may be located between one or more sets of restriction sites. Suitably, the restriction sites may facilitate the integration of the expression cassette into, e.g., a vector, a plasmid, or genomic DNA (e.g. host cell genomic DNA).

For example, an expression cassette of the invention may be transferred from a first polynucleotide sequence, e.g. on a vector, to another by ‘cutting’, e.g. excising, the expression cassette using one or more suitable restriction enzymes and ‘pasting’, e.g. integrating, the expression cassette into a second polynucleotide sequence.

The expression cassette may comprise a polynucleotide of the invention. The expression cassette may comprise a polynucleotide encoding one or more TCRs of the invention. The expression cassette may further comprise an antibiotic resistance gene or other selectable marker gene that allows cells that have successfully integrated the expression cassette into their DNA to be identified. The polynucleotide sequences comprised in the expression cassette may be operably linked to expression control sequences, e.g. a suitable promoter or enhancer sequence. The person skilled in the art will be able to select suitable expression control sequences.

The invention also contemplates a cell expressing a TCR of the invention, which has been engineered to disrupt one or more endogenous MHC genes. Disruption of an endogenous MHC gene can reduce or prevent expression of MHC on the engineered cell surface. Accordingly, such an engineered cell with reduced or no MHC expression will have limited or no capacity to present antigens on its cell surface. Such a cell is particularly advantageous for adoptive cell transfer since the cell will be non-alloreactive, e.g., the cell will not present antigens which could be recognized by the immune system of a subject receiving the adoptively transferred cell. As a result, the transferred cell will not be recognized as ‘non-self’ and an adverse immune reaction to the cell can be avoided. Such a cell is termed a ‘universal cell’ since it is suitable for adoptive transfer to a variety of different hosts regardless of HLA type. The invention provides a method of preparing a non-alloreactive universal T-cell, which expresses a TCR of the invention. Further provided by the invention is a non-alloreactive universal T-cell, which expresses a TCR of the invention.

The invention further contemplates cells which have been engineered to disrupt one or more endogenous genes to modify the cell to enhance advantageous properties, characteristics or functions of the cell and/or reduce undesirable properties, characteristics or functions. For example, by disrupting an endogenous cell the persistence, expansion, activity, resistance to exhaustion/senescence/inhibitory signals, homing capacity, or other cell functions may be modified. As used in this context, the term “modify” may refer to a change in one or more characteristics relative to an equivalent unmodified cell, e.g. a cell in which an endogenous gene has not been disrupted. For example, the change may be an increase, an enhancement or an introduction of a characteristic or function of the cell relative to an equivalent unmodified cell. Alternatively, the change may be a decrease, suppression or abrogation of a characteristic or function of the cell relative to an equivalent unmodified cell.

The polynucleotides and vectors of the invention may be transferred into specific T-cell subsets, including CD4 and/or CD8, naive, memory stem T cells, central memory, effector memory or effector cells, or in other cellular subsets such as to promote different in vivo length of persistence and function in the cells of the invention.

The polynucleotides and vectors of the invention may also be transferred into T-cell subsets with different polarizations, such as Th0/Tc0, Th1/Tc1, Th2/Tc2, Th17, Th22 or others, depending on the cytokine background most appropriate to target a particular tumor type.

Furthermore, the polynucleotides and vectors of the invention encoding the antigen-specific regions of the TCRs of the present invention may be transferred into other cellular subsets, including gamma/delta T-cells, NK cells, NKT cells, cytokine-induced killer (CIK) cells, hematopoietic stem cells or other cells, in order to obtain the therapeutic effect.

### Methods of Preparing a Cell

In one aspect, the present invention provides a method of preparing a cell or a population of cells comprising a TCR, a polynucleotide or a vector according to the present invention.

The polynucleotide or vector of the present invention may be introduced into the cell or population of cells in vitro, ex vivo or in vivo. Suitably, the polynucleotide or vector of the present invention is introduced in vitro or ex vivo.

The polynucleotide or vector of the present invention may be introduced into cells using a variety of techniques known in the art, such as transformation, transfection and transduction.

Several techniques are known in the art, for example transduction with recombinant viral vectors, such as retroviral, lentiviral, adenoviral, adeno-associated viral, baculoviral and herpes simplex viral vectors, Sleeping Beauty vectors; direct injection of nucleic acids and biolistic transformation.

Non-viral delivery systems include but are not limited to transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs), and combinations thereof.

In addition, the invention may employ gene targeting protocols, for example the delivery of DNA-modifying agents.

In one aspect, the present invention provides a method of preparing a cell, which comprises the step of transducing a cell in vitro or ex vivo with a vector of the invention.

In one aspect, the present invention provides a method of preparing a T-cell expressing a TCR of the invention by inducing the differentiation of a stem cell which comprises a polynucleotide or a vector of the invention.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (e.g. CD3, CD4, CD8, CD25, CD127, CD152, CXCR3, or CCR4) may be purified from a starting population of cells. Alternatively, or in addition, a population of cells that does not express another marker may be purified.

By “enriching” a population of cells for a certain type of cells it is to be understood that the concentration of that type of cells is increased within the population. The concentration of other types of cells may be concomitantly reduced. Purification or enrichment may result in the population of cells being substantially pure of other types of cell.

Purifying or enriching for a population of cells expressing a specific marker (e.g. CD3, CD4, CD8, CD25, CD127, CD152, CXCR3, or CCR4) may be achieved by using an agent that binds to that marker, preferably substantially specifically to that marker. An agent that binds to a cellular marker may be an antibody, for example antibody which binds to CD3, CD4, CD8, CD25, CD127, CD152, CXCR3, or CCR4.

The term “antibody” may refer to complete antibodies or antibody fragments capable of binding to a selected target, including Fv, ScFv, F(ab′) and F(ab′)₂, monoclonal and polyclonal antibodies, engineered antibodies including chimeric, CDR-grafted and humanised antibodies, and artificially selected antibodies produced using phage display or alternative techniques. In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification, or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such as biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry. Clinical grade separation may be performed, for example, using the CliniMACS® system (Miltenyi). This is an example of a closed-circuit magnetic bead-based separation technology.

It is also envisaged that dye exclusion properties (e.g. side population or rhodamine labelling) or enzymatic activity (e.g. ALDH activity) may be used to enrich for HSCs.

### Chimeric Molecules

In one aspect, the present invention provides a chimeric molecule comprising a TCR of the invention, a TCR encoded by a polynucleotide of the invention, or a portion thereof, conjugated to a non-cellular substrate.

The conjugation may be covalent or non-covalent. The non-cellular substrate may be a nanoparticle, an exosome, or any non-cellular substrate known in the art. The chimeric molecule of the invention may be soluble.

In one aspect, the present invention provides a chimeric molecule comprising a TCR of the invention, a TCR encoded by a polynucleotide of the invention, or a portion thereof, conjugated to a toxin or an antibody.

The toxin or antibody may be cytotoxic. The toxin may be a cytotoxic molecule or compound, e.g. a radioactive molecule or compound. The TCR portion of the chimeric molecule may confer the ability to recognize cells expressing hTERT or Survivin protein or peptides. Thus, the chimeric molecule may specifically recognize and/or bind to hTERT- or Survivin-expressing tumor cells. Accordingly, the chimeric molecules of the invention may provide hTERT- or Survivin-targeted delivery of cytotoxic toxins, antibodies and/or compounds.

### Pharmaceutical Compositions

In one aspect, the present invention provides a pharmaceutical composition comprising a TCR according to the present invention, a polynucleotide according to the present invention, a vector according to the present invention, a cell or a population of cells according to the present invention, a cell or a population of cells prepared by a method according to the present invention, or a chimeric molecule according to the present invention.

A “pharmaceutical composition” is a composition that comprises or consists of a therapeutically effective amount of a pharmaceutically active agent. A pharmaceutical composition preferably includes a pharmaceutically acceptable carrier, diluent or excipient (including combinations thereof).

By “pharmaceutically acceptable” is included that the formulation is sterile and pyrogen free. The carrier, diluent, and/or excipient must be “acceptable” in the sense of being compatible with the pharmaceutically active agent and not deleterious to the recipients thereof.

Typically, the carriers, diluents, and excipients will be saline or infusion media which will be sterile and pyrogen free, however, other acceptable carriers, diluents, and excipients may be used. Acceptable carriers, diluents, and excipients for therapeutic use are well known in the pharmaceutical art. The choice of pharmaceutical carrier, excipient or diluent can be selected with regard to the intended route of administration and standard pharmaceutical practice. The pharmaceutical compositions may comprise as (or in addition to) the carrier, excipient or diluent any suitable binder(s), lubricant(s), suspending agent(s), coating agent(s) or solubilising agent(s).

The TCR, polynucleotide, vector, cell, population of cells, chimeric molecule, or pharmaceutical composition of the present invention may be administered in a manner appropriate for treating and/or preventing the diseases described herein. Suitable administration routes will be known to the skilled person. The quantity and frequency of administration may be determined by the skilled person, for example depending by such factors as the condition of the subject, and the type and severity of the subject's disease. The pharmaceutical composition may be formulated accordingly.

In one aspect, the present invention provides a pharmaceutical composition comprising the cell or population of cells according to the present invention. The cells of the invention may be formulated for administration to subjects with a pharmaceutically acceptable carrier, diluent or excipient. Suitable carriers and diluents include isotonic saline solutions, for example phosphate-buffered saline, and potentially contain human serum albumin. Handling of the cell therapy products is preferably performed in compliance with FACT-JACIE International Standards for cellular therapy.

The pharmaceutical composition may further comprise one or more other therapeutic agents

The invention further includes kits comprising the TCR, polynucleotide, vector, cell, population of cells, chimeric molecule, or pharmaceutical composition of the present invention. Preferably said kits are for use in the methods and used as described herein, e.g., the therapeutic methods as described herein. Preferably said kits comprise instructions for use of the kit components.

### Methods of Treatment

In one aspect, the present invention provides the TCR, polynucleotide, vector, cell, population of cells, chimeric molecule, or pharmaceutical composition according to the present invention for use as a medicament.

In one aspect, the present invention provides use of the TCR, polynucleotide, vector, cell, population of cells, chimeric molecule, or pharmaceutical composition according to the present invention in the manufacture of a medicament.

In one aspect, the present invention provides a method of administering a therapeutically effective amount of the TCR, polynucleotide, vector, cell, population of cells, chimeric molecule, or pharmaceutical composition according to the present invention to a subject in need thereof.

The TCR, polynucleotide, vector, cell, population of cells, chimeric molecule, or pharmaceutical composition may be administered to any subject in need thereof. The subject may be a mammal. The subject may be a human subject. The human subject may be a child. For example, the child may be less than 10 years in age, less than 9 years in age, less than 8 years in age, less than 7 years in age, less than 6 years in age, less than 5 years in age, less than 4 years in age, less than 3 years in age, or less than 2 years in age. The human subject may be an infant. The subject may be a non-human animal subject.

The subject may have a proliferative disorder. The subject may be at risk of developing a proliferative disorder. The subject may have been previously determined to be at risk of developing a proliferative disorder. The subject may have an increased risk of developing a proliferative disorder. The increased risk may have been determined by genetic screening and/or by reviewing the subject's family history. The subject may express genetic markers indicative of increased risk of developing a proliferative disorder. Suitably, a person skilled in the art will be aware of genetic risk factors (e.g. genetic markers) associated with increased risk of developing a proliferative disorder. The skilled person may be able to use any suitable method or technique known in the art to determine whether the subject has an increased risk of developing a proliferative disorder.

The subject may have previously received treatment for a proliferative disorder. The subject may be in remission. The subject may be resistant to chemotherapy.

The subject may have been previously determined to be in need of a TCR, polynucleotide, vector, cell, population of cells, chimeric molecule, or pharmaceutical composition according to the present invention on the basis of expression of hTERT or Survivin. For example, the subject may have a cell population that exhibits increased expression of hTERT or Survivin relative to a healthy control cell population. A variety of techniques known in the art may be used to determine hTERT or Survivin expression, for example quantitative RT-PCR can be used to determine the amount of RNA transcript, which is indicative of protein expression. The person skilled in the art will also appreciate that protein expression may be determined by performing western blots using commercially available antibodies. The subject may also have been previously identified as having an alteration (e.g. mutation or deletion) in a hTERT or Survivin gene. Such an alteration may be hereditary.

### Proliferative Disorders

The TCR, polynucleotide, vector, cell, population of cells, chimeric molecule, or pharmaceutical composition according to the present invention may be used to prevent and/or treat a proliferative disorder.

In one aspect, the present invention provides the TCR, polynucleotide, vector, cell, population of cells, chimeric molecule, or pharmaceutical composition according to the present invention for use in preventing and/or treating a proliferative disorder.

In one aspect, the present invention provides use of the TCR, polynucleotide, vector, cell, population of cells, chimeric molecule, or pharmaceutical composition according to the present invention in the manufacture of a medicament for preventing and/or treating a proliferative disorder.

In one aspect, the present invention provides a method of preventing and/or treating a proliferative disorder, the method comprising administering a therapeutically effective amount of the TCR, polynucleotide, vector, cell, population of cells, chimeric molecule, or pharmaceutical composition according to the present invention to a subject in need thereof.

As used herein, the term “preventing” may refer to averting, delaying, impeding or hindering the contraction of the disease. The treatment may, for example, prevent or reduce the likelihood of developing or contracting a proliferative disorder.

As used herein, the term “treating” may refer to caring for a diseased subject, in order to ameliorate, cure or reduce the symptoms of the disease, or in order to reduce, halt or delay the progression of the disease.

The proliferative disorder may be any proliferative disorder associated with hTERT or Survivin expression. Any suitable method may be used to determine such proliferative disorders (see e.g. Tang, Z., et al., 2017. Nucleic acids research, 45(W1), pp. W98-W102). Transcriptional regulation of hTERT is believed to play a major role in telomerase activation in human cancers (see e.g. Leão, R., et al., 2018. Journal of biomedical science, 25(1), pp. 1-12) and Survivin is highly expressed in most cancers (see e.g. Jaiswal, P. K., et al., 2015. The Indian journal of medical research, 141(4), pp. 389-397).

The proliferative disorder may be a hematological malignancy or a solid tumor.

As used herein, a “hematological malignancy” may refer to a cancer that affects the blood, bone marrow, and lymph nodes. The hematological malignancy may be selected from the group consisting of acute myeloid leukemia (AML), chronic myeloid leukemia (CML), lymphoblastic leukemia, acute lymphocytic leukemia (ALL), myelodisplastic syndromes, lymphoma, multiple myeloma, non-Hodgkin lymphoma, and Hodgkin lymphoma.

As used herein, a “solid tumour” may refer to an abnormal mass of tissue that usually does not contain cysts or liquid areas. The solid tumor may be selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, oral cancer, hepatocellular carcinoma, bladder cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.

The proliferative disorder may be selected from a group consisting of acute myeloid leukemia (AML), chronic myeloid leukemia (CML), lymphoblastic leukemia, acute lymphocytic leukemia (ALL), myelodisplastic syndromes, lymphoma, multiple myeloma, non-Hodgkin lymphoma, and Hodgkin lymphoma, lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, oral cancer, hepatocellular carcinoma, bladder cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.

In some embodiments, the proliferative disorder is acute myeloid leukemia (AML) or chronic myeloid leukemia (CML). In some embodiments, the proliferative disorder is acute myeloid leukemia (AML).

In some embodiments, the method for treating and/or preventing a proliferative disorder comprises the step of administering a chemotherapy to the subject. The chemotherapy may be administered to the subject simultaneously, sequentially or separately with the TCR, polynucleotide, vector, cell, population of cells, chimeric molecule, or pharmaceutical composition of the invention.

The practice of the invention will employ, unless otherwise indicated, conventional techniques of cell biology, molecular biology, histology, immunology, oncology, which are within the capabilities of a person of ordinary skill in the art. Such techniques are explained in the literature.

See, for example: Skoog, D. A., et al. (2013) Fundamentals of Analytical Chemistry, 9th edition, Cengage learning; Walker J. M. (2009) The Protein Protocols Handbook, 3rd edition, Springer Nature; Green, M. R. and Sambrook, J. (2012) Molecular Cloning: A Laboratory Manual, 4th Edition, Cold Spring Harbor Laboratory Press; Ausubel, F. M., et al. (2003) Current Protocols in Molecular Biology, John Wiley & Sons; Hill, A. J. (2013) DNA Sequencing Protocols, Humana Press; Nielsen, B. S. and Jones, J. (2021) In Situ Hybridization Protocols, Springer U S; Herdewijn, P. (2010) Oligonucleotide Synthesis: Methods and Applications, Humana Press; and Luo, Y. (2019) CRISPR Gene Editing: Methods and Protocols, Springer New York. Each of these general texts is herein incorporated by reference.

### EXAMPLES

The invention will now be further described by way of Examples, which are meant to serve to assist one of ordinary skill in the art in carrying out the invention and are not intended in any way to limit the scope of the invention.

### Example 1—Isolation of Tumour-Specific TCRs

We sorted and activated hTERT and Survivin—specific T cells from two patients affected by blood malignancies who underwent Allogeneic hematopoientic Stem Cell transplantation (Allo-HSCT) and who showed circulating tumor-associated antigen (TAA)-specific T cells.

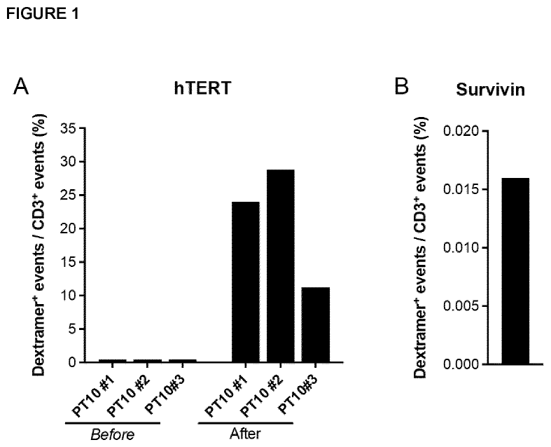

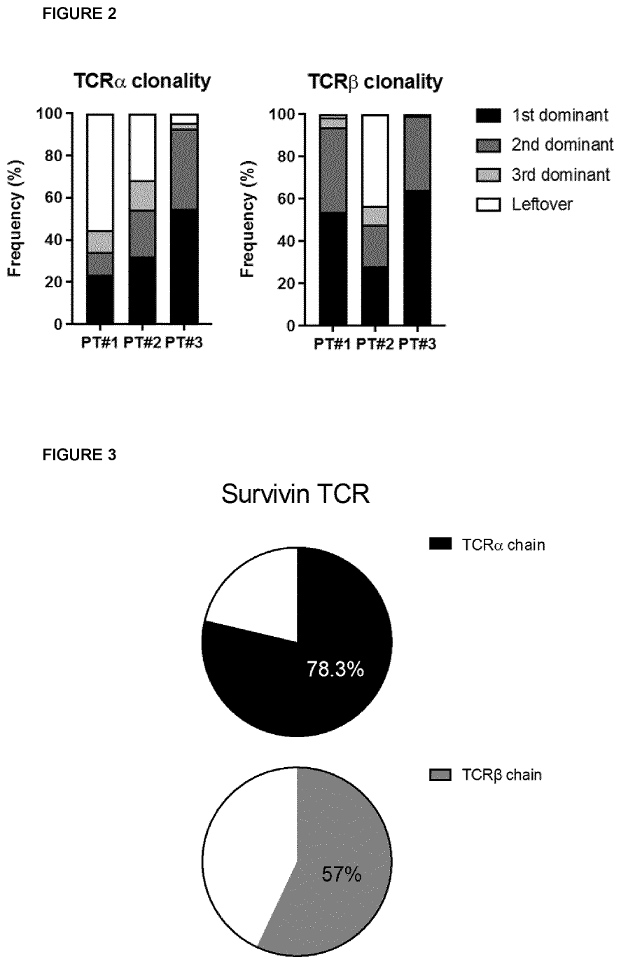

Sorting was performed by using Dextramers restricted to hTERT₆₆₃₋₆₇₂ HLA-A*0301 and Survivin₉₆₋₁₀₄ (modified form) HLA-A*0201. Three hTERT-specific and one Survivin-specific cell culture were established (FIG. 1). TCR sequencing showed how a limited amount of dominant clones described the cell cultures, with reduced clonal diversity compared to ex vivo, allowing dominant TCR reconstruction (FIGS. 2-3).

One dominant TCR was reconstructed from each of two of the hTERT-specific cell cultures (PT #1 and PT #2) and are referred to herein as hTERT #1 and hTERT #2, two dominant TCRs were reconstructed from one of the hTERT-specific cell cultures (PT #3) and referred to herein as hTERT #3 and hTERT #4, and one dominant TCR was constructed from the Survivin-specific cell culture.

The sequences for each of the dominant TCRs are shown above as follows:
hTERT #1 TCR sequences: SEQ ID NOs: 6-16 and 63-67
hTERT #2 TCR sequences: SEQ ID NOs: 17-27 and 68-72
hTERT #3 TCR sequences: SEQ ID NOs: 28-38 and 73-77
hTERT #4 TCR sequences: SEQ ID NOs: 39-49 and 78-82
Survivin TCR sequences: SEQ ID NOs: 52-62 and 83-87

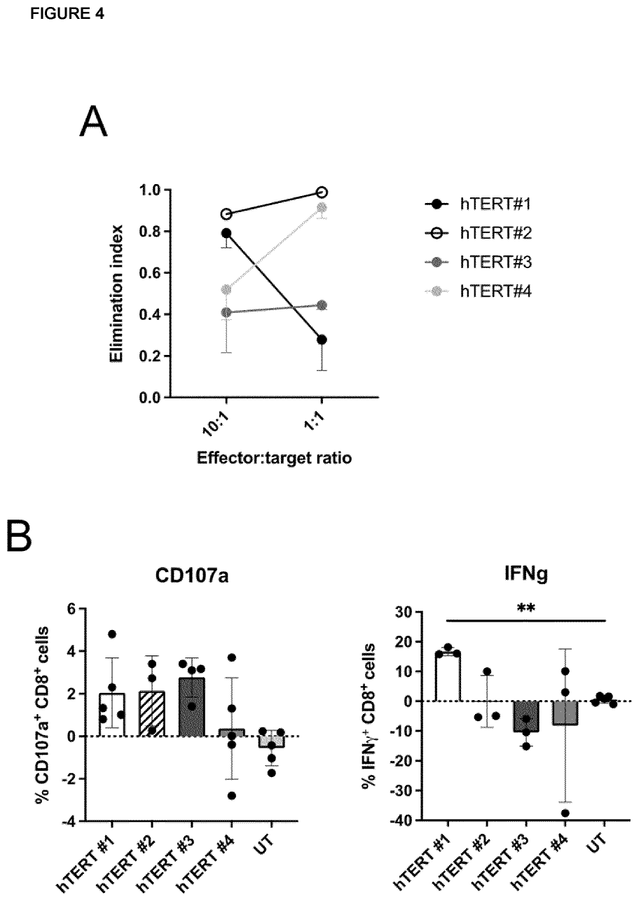

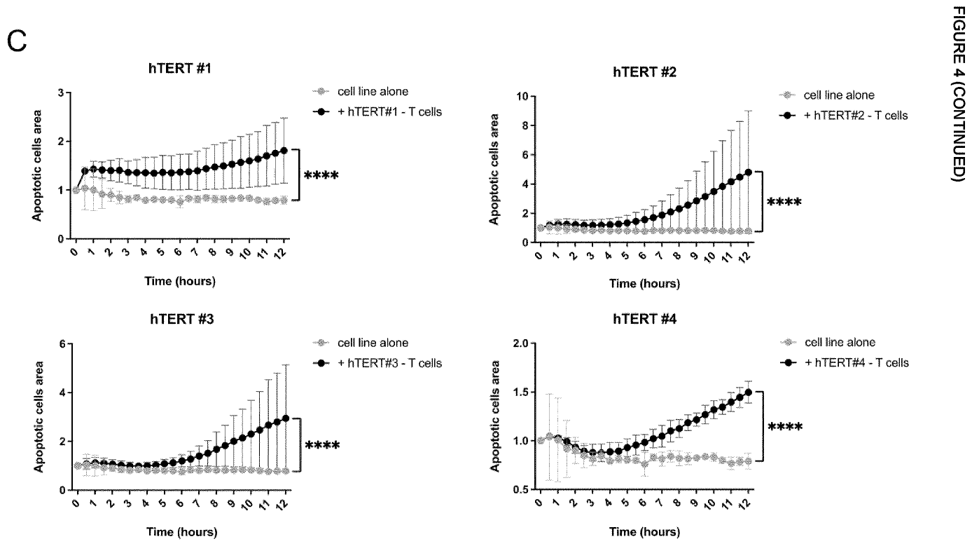

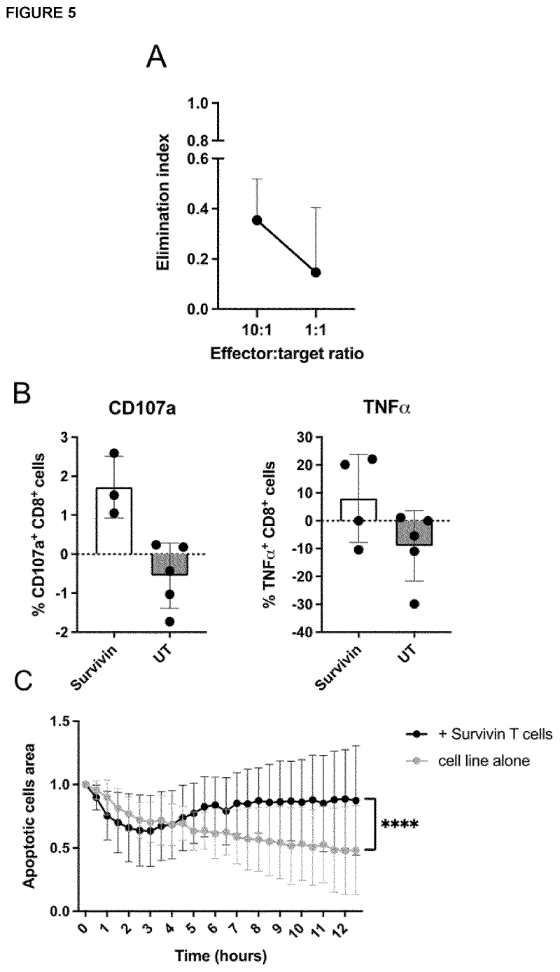

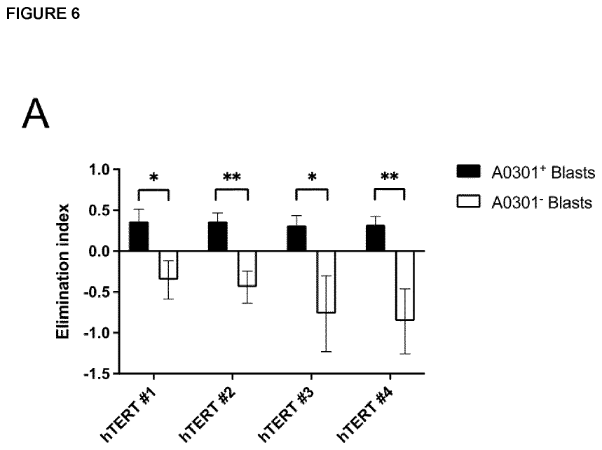

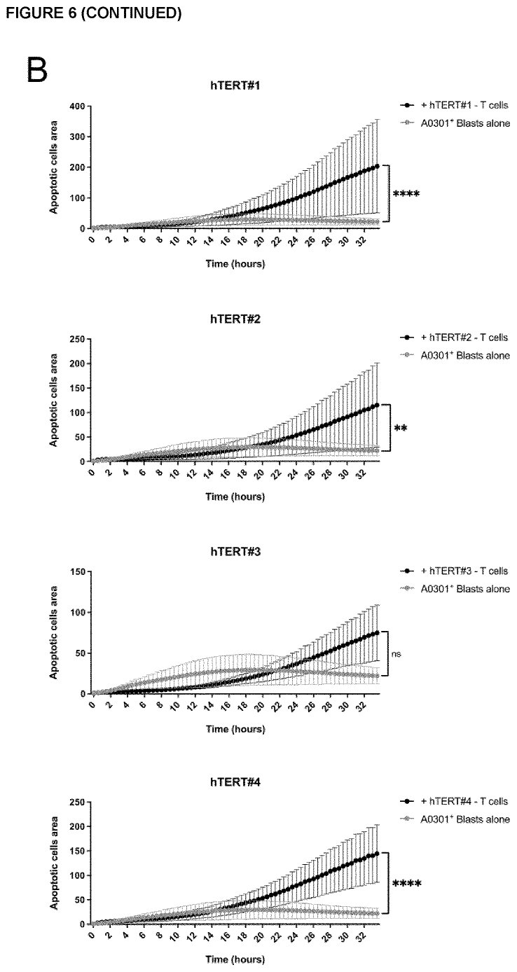

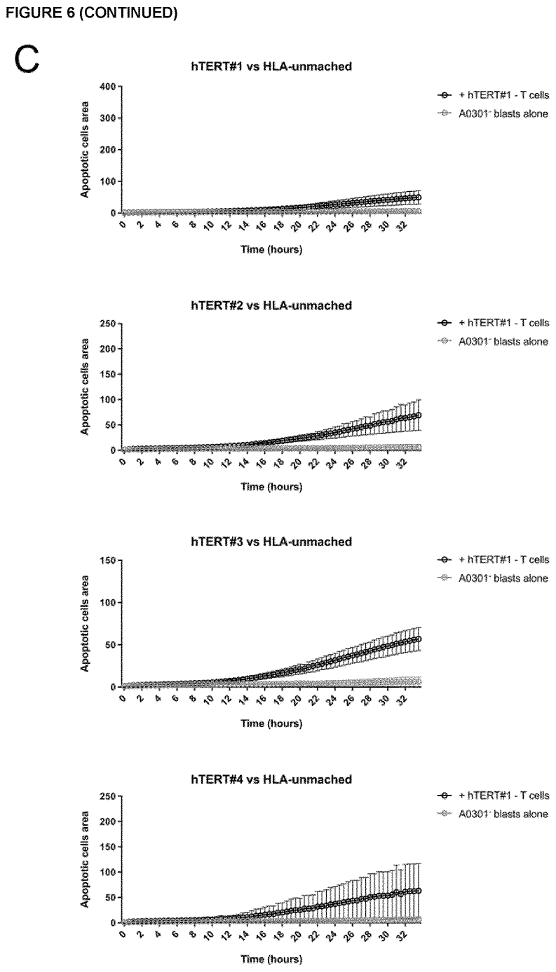

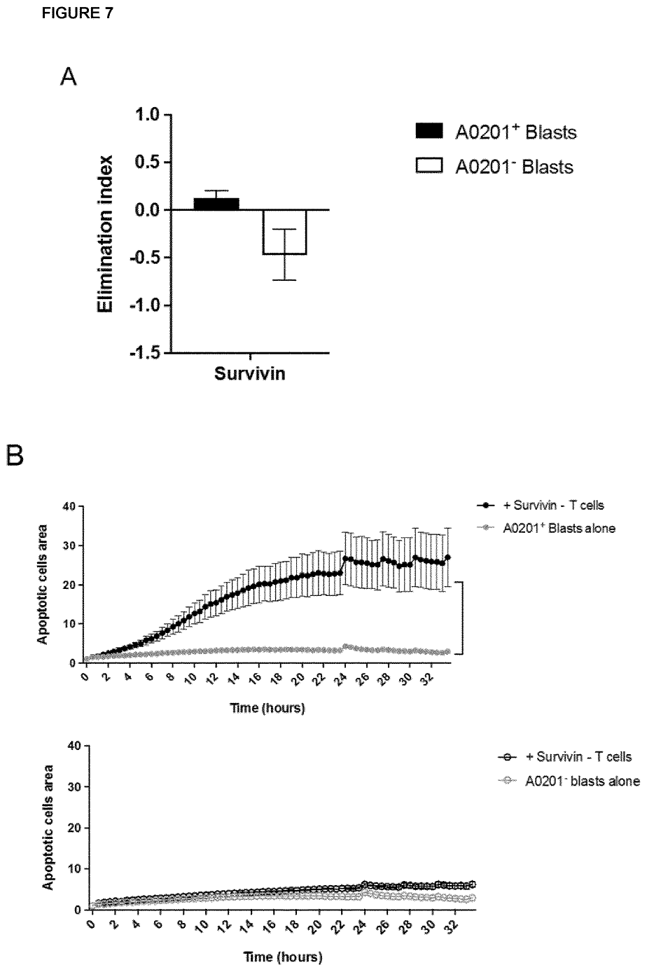

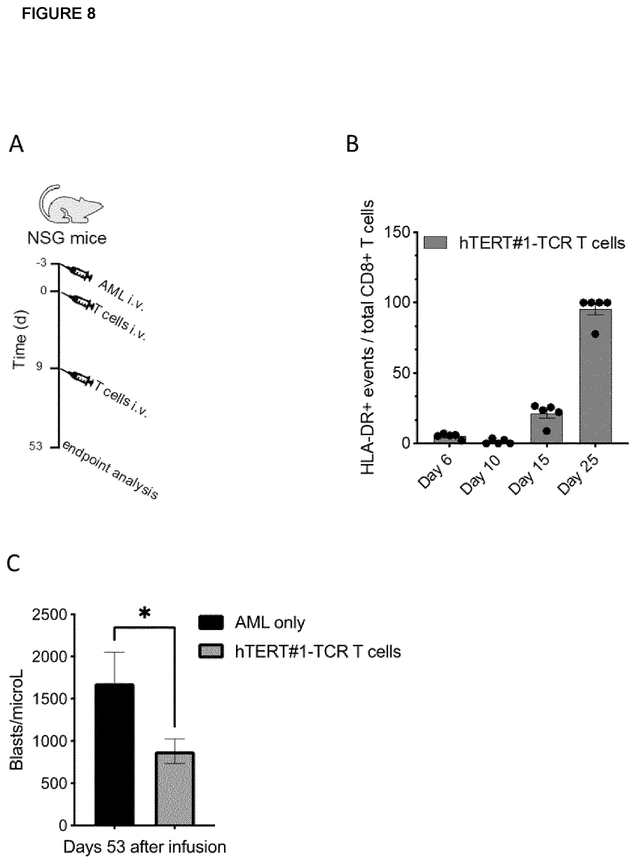

We next reconstructed the dominant TCRs reactive against Survivin and hTERT epitopes and engineered them into TCR-KO donor T cells derived from healthy donors to generate TCR-edited cells. TCR-edited T cells were then challenged with tumor cell lines pulsed with the appropriate peptides. TCR-edited T cells degranulated and mediated peptide-specific target killing (FIGS. 4-5).

We then challenged edited T cells against primary AML blasts. Edited T cells were co-cultured with Survivin- or hTERT-overexpressing primary AML blasts, either exhibiting (target blasts) or not (control blasts) the relevant HLA restriction (HLA-A*0201⁺ targets for Survivin₉₆₋₁₀₄; HLA-A*0301⁺ targets for hTERT₆₆₃₋₆₇₂). Apoptosis was induced and target blasts were eliminated by the engineered cellular products (FIGS. 6-7).

We developed a pipeline for the isolation of TAA-specific TCRs directed against immunodominant hTERT and Survivin peptides. When the isolated TAA-specific TCRs were expressed on healthy T cells, specific in vitro killing was observed for hTERT and Survivin TCR-edited T cells, underscoring the functionality of these TCRs. With this approach, we have widened the array of TCRs and target epitopes in blood malignancies.

### Example 2—Materials and Methods

### Isolation of Peripheral Blood Mononuclear Cells (PBMCs)

Peripheral blood was obtained from patients affected by blood malignancies who underwent Allogeneic hematopoientic Stem Cell transplantation (Allo-HSCT). All samples were collected under written informed consent in agreement with the Declaration of Helsinki, previous approval by the Institutional Ethical Committee.

Isolation and Expansion of hTERT-Specific T Cells and Survivin-Specific T Cells

Peripheral blood mononuclear cells (PBMCs) were isolated via fycoll hypaque gradient centrifugation (1700 rmp, 30 mins). Successively, PBMCs were incubated with Ammonium-Chloride-Potassium solution and then washed multiple times with PBS supplemented with 2% FBS to eliminate erythrocytes and platelets. Freshly isolated PBMCs from patients who underwent allo-HSCT were resuspended in PBS supplemented with 5% FBS and 5 μM Dasatinib (Axon Medchem, VA) and incubated 15 minutes in incubator (37° C. and 5% CO2) to enhance the expression of TCRs in T cells.

Cells were labeled with hTERT—Dextramer (Immudex) conjugated with PE fluorochrome and specific for hTERT₆₆₃₋₆₇₂ HLA-A*0301 or Survivin₉₆₋₁₀₄ (modified form) HLA-A*0201 restricted peptide. Cells were sorted using anti-PE magnetic beads (Biolegend). The positive fraction was plated in 10-20 wells of a 96 multiwell plate, which were previously coated with anti-CD3 and anti-CD28 mAb, and maintained in X-VIVO with 5% FBS, penicillin-streptomycin (Euroclone), 2 mM glutamine, 5 ng/ml IL-7, 5 ng/ml IL-15. T cell specific expansion was assessed by dextramer binding at flow cytometry.

### Assessment of T Cell Clonality

We used the IO Test Beta Mark TCR V beta repertoire kit (Beckman Coulter) to determine the TCR VB repertoire of expanded TAA-specific T cells. The kit allows the evaluation of the surface expression of 24 different VB chains, which represent the 75% of the complete human repertoire.

### TCR Repertoire Sequencing

RNA samples were collected at each round of stimulation to perform TCR sequencing. RNA was extracted by using Arcturus Pico Pure RNA extraction kit. Complementarity determining region (CDR) 3 sequences of all different antigen-specific T cells were amplified by using a modified RACE approach (Ruggiero, E. et al. Nat. Comm. 6,8081 (2015)). Samples were sequenced by using an IlluminaMiSeq sequencer and CDR3 clonotypes identified using MiXCR (Bolotin, D., et al. 2015. Nat Methods 12, 380-381).

### TCR Reconstruction and Lentiviral Packaging

Newly identified α and β TCR chain sequences were codon-optimized, synthesized (Twist Bioscience) and then inserted into plasmid vectors under a bidirectional human phosphoglycerate kinase (PGK) promoter and human minimal CMV promoter (mhCMV). Plasmids were packaged into lentiviral vectors as integrase-competent third-generation constructs pseudo-typed by the vesicular stomatitis virus (VSV) envelope. The TCR was transferred into T cells using the prepared lentiviral vectors into TCR-KO T cells.

### T Cell Receptor Editing

PBMC harvested from healthy donors were activated using anti-CD3/anti-CD28-coated magnetic beads (ClinExVivo CD3/CD28; Invitrogen) and maintained at a concentration of 10⁶ cells/mL in complete X-vivo supplemented with IL-7+IL-15 (5 ng/ml each). Two days after stimulation, T cells were electroporated with RNP complexes (consisting of purified Spy Cas9 nuclease duplexed with synthetic gRNAs) targeting the TRAC and the TRBC1/2 loci simultaneously using the Lonza Nucleofector 4D Electroporation System. The day after, T cells were transduced with a LV encoding for the tumor-specific TCR of interest. At day 6 post stimulation, beads were removed from culture and between day 14 to 21 tested in co-culture assay or frozen for later in vivo studies.

### In Vitro Co-Culture Assays

A total of 100.000 effector T cells per condition were resuspended in X-vivo supplemented with 5% FBS, 1% P/S and 1% glutamine. Target cells were added, at target:effector ratios between 1:1 and 1:100, according to the experimental layout. In the experiments employing peptide-pulsed tumor cell lines, target cells were incubated overnight with the appropriate peptide (1 μg/ml) in RPMI (Euroclone) supplemented with 5% FBS, 1% P/S and 1% glutamine, before plating them with effectors. For evaluating cytokine release on target cells, plates were kept in culture for 6 hours at 37° C. and 5% CO2. After incubation, cells were first stained with surface molecules (15 mins, RT), then fixed (FoxP3 Fix/Perm buffer (Biolegend), 20 mins) and permeabilized (FoxP3 Perm buffer (Biolegend, 20 mins), according to the manufacturer's instructions. Samples were then stained for cytokines at RT for 15 mins.

For killing assay cells were plated and kept at 37° C. and 5% CO2 after the addition of anti-CD28 monoclonal antibodies (1 μg/ml; BD biosciences) for 72 hours in case of co-culture with cell lines, and for 24 hours for co-cultures with primary AML blasts. After incubations cells were stained with the appropriate fluorochromes (15 minutes at room temperature) and then washed. Viable target cells were counted using counting beads (Flow-count fluorospheres, Beckman Coulter), according to manufacturer's instructions. Elimination index was calculated according to the formula: [1-(number of alive target cells in culture with effector T cells/number of alive target cells in control culture)]. Samples were acquired at the institutional BD CANTO™ or CytoFLEX™ (thermoFisher) flow cytometers. Data were analyzed by FlowJo software version 10 (BD).

### Live-Cell Imaging Assays

Target cells were plated for co-culture assays as described above. Target cells were labeled with Incucyte® NucLight Rapid Red Reagent (Sartorius) according to manufacturer's instructions and then resuspended in X-vivo supplemented with 5% FBS, 1% P/S and 1% glutamine, anti-CD28 monoclonal antibodies (1 μg/ml) and Incucyte® Caspase-3/7 Green Apoptosis Assay Reagent (Sartorius). Effector T cells were then added (100.000 cells/well). The plate was analyzed at the institutional Incucyte® incubator cell imager and built-in analytical software.

### Example 3—In Vivo Studies Using hTERT-TCR T Cells

A hTERT-specific TCR (hTERT #1) was selected for further in vivo testing against primary AML blasts (FIG. 8A). hTERT #1 TCR-engineered T cells expanded and expressed the activation marker HLA-DR (FIG. 8B) upon infusion into NOD-SCID interleukin (IL)-2Rgamma(null) (NSG) mice while delaying tumor expansion (FIG. 8C).

### Materials and Methods

For the in vivo assessment of anti-tumor efficacy in the acute myeloid leukemia context we employed 6- to 9-week-old female non-irradiated immunodeficient NSG mice from Jackson Labs. The experimental protocol was approved by the San Raffaele Institutional Animal Care and Use Committee and by the Italian Ministry of Health.

Primary AML blasts were harvested after informed consent from one HLA-A*03:01 hTERT+AML patient and infused intravenously (2.5×10⁶ cells). Three and 12 days later, mice were treated with engineered T lymphocytes expressing the hTERT #1-specific TCR (10×10⁶ hTERT #1 TCR-engineered cells). Mice infused only with tumor cells (AML only) where used as a control.

Animals were monitored 2-3 times a week for Graft-versus-Host Disease (GvHD) or leukemia occurrence. Human chimerism and acute myeloid leukemia occurrence were assessed by flow cytometry analysis. Briefly, 50 μL of mouse peripheral blood, previously treated with heparin, were incubated with 3 mL ACK lysis buffer for 10 min at room temperature to eliminate erythrocytes. Cells were pelleted by centrifugation, washed with FACS buffer and stained with a viability dye and with the mixture of antibodies for 15 min at room temperature. Afterwards, cells were washed and re-suspended in FACS buffer. Viable T lymphocytes and target cells were counted by using counting beads (Flow-count fluorospheres, Beckman Coulter), according to the manufacturer's instructions. Flow cytometry data were acquired at the BD FACS Canto II (BD Biosciences). For data collection the BD FACS DIVA Software (BD Biosciences, Franklin Lakes, NJ, USA) was used and data analysis was performed using FlowJo software (Tree star Inc).

### Embodiments

Various preferred features and embodiments of the present invention will now be described with reference to the following numbered paragraphs (paras).
1. A T-cell receptor (TCR) which binds to an immunogenic peptide when presented by a major histocompatibility complex (MHC), wherein:
   (i) the immunogenic peptide is a Human Telomerase Reverse Transcriptase (hTERT) peptide and the TCR comprises a CDR3α comprising the amino acid sequence of CADWVDMRF (SEQ ID NO: 8) or a variant thereof having up to three amino acid substitutions, additions or deletions, and a CDR3β comprising the amino acid sequence of CSAPLDRGSNQPQHF (SEQ ID NO: 13) or a variant thereof having up to three amino acid substitutions, additions or deletions;
   (ii) the immunogenic peptide is a Human Telomerase Reverse Transcriptase (hTERT) peptide and the TCR comprises a CDR3α comprising the amino acid sequence of CAVSRPNSGYSTLTF (SEQ ID NO: 19) or a variant thereof having up to three amino acid substitutions, additions or deletions, and a CDR3β comprising the amino acid sequence of CASSVRTPSGQETQYF (SEQ ID NO: 24) or a variant thereof having up to three amino acid substitutions, additions or deletions;
   (iii) the immunogenic peptide is a Human Telomerase Reverse Transcriptase (hTERT) peptide and the TCR comprises a CDR3α comprising the amino acid sequence of CAVETGGGATNKLIF (SEQ ID NO: 30) or a variant thereof having up to three amino acid substitutions, additions or deletions, and a CDR3β comprising the amino acid sequence of CASSEFWLTQETQYF (SEQ ID NO: 35) or a variant thereof having up to three amino acid substitutions, additions or deletions;
   (iv) the immunogenic peptide is a Human Telomerase Reverse Transcriptase (hTERT) peptide and the TCR comprises a CDR3α comprising the amino acid sequence of CAVSHGRGGATNKLIF (SEQ ID NO: 41) or a variant thereof having up to three amino acid substitutions, additions or deletions, and a CDR3β comprising the amino acid sequence of CASDRVLGYEQYF (SEQ ID NO: 46) or a variant thereof having up to three amino acid substitutions, additions or deletions; or
   (v) the immunogenic peptide is a Survivin peptide and the TCR comprises a CDR3α comprising the amino acid sequence of CALDRMDSSYKLIF (SEQ ID NO: 54) or a variant thereof having up to three amino acid substitutions, additions or deletions, and a CDR3β comprising the amino acid sequence of CASSYDQDGEAFF (SEQ ID NO: 59) or a variant thereof having up to three amino acid substitutions, additions or deletions.
2. The TCR according to para 1, which binds to a Human Telomerase Reverse Transcriptase (hTERT) peptide when presented by an MHC, wherein the hTERT peptide comprises or consists of the amino acid sequence of SVLNYERARR (SEQ ID NO: 5) or a variant thereof having up to three amino acid substitutions, additions or deletions.
3. A T-cell receptor (TCR) which binds to a Human Telomerase Reverse Transcriptase (hTERT) peptide when presented by an MHC, wherein the hTERT peptide comprises or consists of the amino acid sequence of SVLNYERARR (SEQ ID NO: 5) or a variant thereof having up to three amino acid substitutions, additions or deletions.
4. The TCR according to para 3, wherein the TCR comprises:
   (i) a CDR3α comprising the amino acid sequence of CADWVDMRF (SEQ ID NO: 8) or a variant thereof having up to three amino acid substitutions, additions or deletions, and a CDR3β comprising the amino acid sequence of CSAPLDRGSNQPQHF (SEQ ID NO: 13) or a variant thereof having up to three amino acid substitutions, additions or deletions;
   (ii) a CDR3α comprising the amino acid sequence of CAVSRPNSGYSTLTF (SEQ ID NO: 19) or a variant thereof having up to three amino acid substitutions, additions or deletions, and a CDR3β comprising the amino acid sequence of CASSVRTPSGQETQYF (SEQ ID NO: 24) or a variant thereof having up to three amino acid substitutions, additions or deletions;
   (iii) a CDR3α comprising the amino acid sequence of CAVETGGGATNKLIF (SEQ ID NO: 30) or a variant thereof having up to three amino acid substitutions, additions or deletions, and a CDR3β comprising the amino acid sequence of CASSEFWLTQETQYF (SEQ ID NO: 35) or a variant thereof having up to three amino acid substitutions, additions or deletions; or
   (iv) a CDR3α comprising the amino acid sequence of CAVSHGRGGATNKLIF (SEQ ID NO: 41) or a variant thereof having up to three amino acid substitutions, additions or deletions, and a CDR3β comprising the amino acid sequence of CASDRVLGYEQYF (SEQ ID NO: 46) or a variant thereof having up to three amino acid substitutions, additions or deletions.
5. The TCR according to any of paras 1-4, wherein the TCR comprises the following CDR sequences:
   (i) CDR1α—DSASNY (SEQ ID NO: 6), CDR2α—IRSNVGE (SEQ ID NO: 7), CDR3α-CADWVDMRF (SEQ ID NO: 8), CDR1β—DFQATT (SEQ ID NO: 11), CDR2β—SNEGSKA (SEQ ID NO: 12), and CDR3β—CSAPLDRGSNQPQHF (SEQ ID NO: 13), or variants thereof each having up to three amino acid substitutions, additions or deletions;
   (ii) CDR1α—TSGFNG (SEQ ID NO: 17), CDR2α—NVLDGL (SEQ ID NO: 18), CDR3α—CAVSRPNSGYSTLTF (SEQ ID NO: 19), CDR1β—PRHDT (SEQ ID NO: 22), CDR2β—FYEKMQ (SEQ ID NO: 23), and CDR3β—CASSVRTPSGQETQYF (SEQ ID NO: 24), or variants thereof each having up to three amino acid substitutions, additions or deletions;
   (iii) CDR1α—VSNAYN (SEQ ID NO: 28), CDR2α—GSKP (SEQ ID NO: 29), CDR3α—CAVETGGGATNKLIF (SEQ ID NO: 30), CDR1β—MNHNS (SEQ ID NO: 33), CDR2β—SASEGT (SEQ ID NO: 34), and CDR3β—CASSEFWLTQETQYF (SEQ ID NO: 35), or variants thereof each having up to three amino acid substitutions, additions or deletions; or
   (iv) CDR1α—DSVNN (SEQ ID NO: 39), CDR2α—IPSGT (SEQ ID NO: 40), CDR3α—CAVSHGRGGATNKLIF (SEQ ID NO: 41), CDR1β—MNHNS (SEQ ID NO: 44), CDR2β—SASEGT (SEQ ID NO: 45), and CDR3β—CASDRVLGYEQYF (SEQ ID NO: 46), or variants thereof each having up to three amino acid substitutions, additions or deletions.
6. The TCR according to any of paras 1 to 5, wherein the TCR comprises:
   (i) an α chain variable domain comprising the amino acid sequence of SEQ ID NO: 9 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 14 or a variant thereof having at least 70%, at least 75%, at least 80%, at least 85%, at least 90%, at least 95%, at least 98%, or at least 99% sequence identity thereto;
   (ii) an α chain variable domain comprising the amino acid sequence of SEQ ID NO: 20 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 25 or a variant thereof having at least 70%, at least 75%, at least 80%, at least 85%, at least 90%, at least 95%, at least 98%, or at least 99% sequence identity thereto;
   (iii) an α chain variable domain comprising the amino acid sequence of SEQ ID NO: 31 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 36 or a variant thereof having at least 70%, at least 75%, at least 80%, at least 85%, at least 90%, at least 95%, at least 98%, or at least 99% sequence identity thereto; or
   (iv) an α chain variable domain comprising the amino acid sequence of SEQ ID NO: 42 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 47 or a variant thereof having at least 70%, at least 75%, at least 80%, at least 85%, at least 90%, at least 95%, at least 98%, or at least 99% sequence identity thereto.
7. The TCR according to any of paras 1 to 6, wherein the TCR comprises:
   (i) an α chain comprising the amino acid sequence of SEQ ID NO: 10 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 15 or 16, or variants thereof having at least 70%, at least 75%, at least 80%, at least 85%, at least 90%, at least 95%, at least 98%, or at least 99% sequence identity thereto;
   (ii) an α chain comprising the amino acid sequence of SEQ ID NO: 21 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 26 or 27, or variants thereof having at least 70%, at least 75%, at least 80%, at least 85%, at least 90%, at least 95%, at least 98%, or at least 99% sequence identity thereto;
   (iii) an α chain comprising the amino acid sequence of SEQ ID NO: 32 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 37 or 38, or variants thereof having at least 70%, at least 75%, at least 80%, at least 85%, at least 90%, at least 95%, at least 98%, or at least 99% sequence identity thereto; or
   (iv) an α chain comprising the amino acid sequence of SEQ ID NO: 43 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 48 or 49, or variants thereof having at least 70%, at least 75%, at least 80%, at least 85%, at least 90%, at least 95%, at least 98%, or at least 99% sequence identity thereto.
8. The TCR according to any of paras 1 to 7, wherein the TCR is restricted to HLA-A*0301.
9. A T-cell receptor (TCR) which binds to Survivin peptide when presented by a major histocompatibility complex (MHC), wherein the TCR comprises a CDR3α comprising the amino acid sequence of CALDRMDSSYKLIF (SEQ ID NO: 54) or a variant thereof having up to three amino acid substitutions, additions or deletions, and a CDR3β comprising the amino acid sequence of CASSYDODGEAFF (SEQ ID NO: 59) or a variant thereof having up to three amino acid substitutions, additions or deletions.
10. The TCR according to para 1 or 9, wherein the TCR comprises the following CDR sequences: CDR1α—NYSPAY (SEQ ID NO: 52), CDR2α—IRENEKE (SEQ ID NO: 53), CDR3α—CALDRMDSSYKLIF (SEQ ID NO: 54), CDR1β—MNHEY (SEQ ID NO: 57), CDR2β—SVGAGI (SEQ ID NO: 58), and CDR3β—CASSYDQDGEAFF (SEQ ID NO: 59), or variants thereof each having up to three amino acid substitutions, additions or deletions.
11. The TCR according to para 1 or 9-10, wherein the TCR comprises the following CDR sequences: CDR1α—NYSPAY (SEQ ID NO: 52), CDR2α—IRENEKE (SEQ ID NO: 53), CDR3α—CALDRMDSSYKLIF (SEQ ID NO: 54), CDR1β—MNHEY (SEQ ID NO: 57), CDR2β—SVGAGI (SEQ ID NO: 58), and CDR3β—CASSYDQDGEAFF (SEQ ID NO: 59).
12. The TCR according to any of paras 1 or 9-11, wherein the TCR comprises an α chain variable domain comprising the amino acid sequence of SEQ ID NO: 55 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 60 or a variant thereof having at least 70%, at least 75%, at least 80%, at least 85%, at least 90%, at least 95%, at least 98%, or at least 99% sequence identity thereto.
13. The TCR according to any of paras 1 or 9-12, wherein the TCR comprises an α chain comprising the amino acid sequence of SEQ ID NO: 56 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 61 or 62, or variants thereof having at least 70%, at least 75%, at least 80%, at least 85%, at least 90%, at least 95%, at least 98%, or at least 99% sequence identity thereto.
14. The TCR according to any of paras 1 or 9-13, wherein the TCR is restricted to HLA-A*0201.
15. The TCR according to any of paras 1 or 9-14, wherein the Survivin peptide comprises or consists of the amino acid sequence of LMLGEFLKL (SEQ ID NO: 51) or a variant thereof having up to three amino acid substitutions, additions or deletions.
16. The TCR according to any preceding para, wherein the TCR comprises one or more mutations at the α chain/β chain interface, such that when the α chain and the β chain are expressed in a T-cell, the frequency of mispairing between said chains and endogenous TCR α and β chains is reduced.
17. The TCR according to para 16, wherein the one or more mutations introduce a cysteine residue into the constant region domain of each of the α chain and the β chain, wherein the cysteine residues are capable of forming a disulphide bond between the α chain and the β chain.
18. The TCR according to any preceding para, wherein the TCR comprises a murinised constant region.
19. The TCR according to any preceding para, wherein the TCR is a soluble TCR.
20. A polynucleotide encoding the α chain and/or the β chain of a TCR according to any preceding para.
21. The polynucleotide according to para 20, wherein the polynucleotide encodes the α chain linked to the β chain.
22. The polynucleotide according to para 20 or 21, wherein the polynucleotide further encodes one or more short interfering RNA (siRNA) or other agents capable of reducing or preventing expression of one or more endogenous TCR genes.
23. A vector comprising a polynucleotide according to any of paras 20-22.
24. The vector according to para 23, wherein the vector is a plasmid or a viral vector.
25. The vector according to para 23 or 24, wherein the vector comprises a polynucleotide which encodes one or more CD3 chains, CD8, a suicide gene and/or a selectable marker.
26. A cell comprising a TCR according to any of paras 1-19, a polynucleotide according to any of paras 20-22 or a vector according to any of paras 23-25.
27. The cell according to para 26, wherein the cell further comprises a vector which encodes one or more CD3 chains, CD8, a suicide gene and/or a selectable marker.
28. The cell according to para 26 or 27, wherein the cell is a T-cell, a lymphocyte, or a stem cell.
29. The cell according to para 28, wherein the T-cell, the lymphocyte, or the stem cell is selected from the group consisting of CD4+ cells, CD8+ cells, naive T-cells, memory stem T-cells, central memory T-cells, double negative T-cells, effector memory T-cells, effector T-cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, cytokine-induced killer (CIK) cells, hematopoietic stem cells and pluripotent stem cells.
30. The cell according to any of paras 26 to 29, wherein the cell is a T-cell, optionally wherein the cell is a T-cell which has been isolated from a subject.
31. The cell according to any of paras 26 to 30, wherein an endogenous gene encoding a TCR α chain and/or an endogenous gene encoding a TCR β chain is disrupted, preferably such that the endogenous gene encoding a TCR α chain and/or the endogenous gene encoding a TCR β chain is not expressed, optionally wherein the endogenous gene encoding a TCR α chain and/or the endogenous gene encoding a TCR β chain is disrupted by insertion of an expression cassette comprising a polynucleotide sequence encoding the TCR of any of paras 1-19.
32. The cell according to any of paras 26 to 31, wherein one or more endogenous genes encoding an MHC is disrupted.
33. The cell according to any of paras 26 to 32, wherein an endogenous gene involved in persistence, expansion, activity, resistance to exhaustion/senescence/inhibitory signals, homing capacity, or other T-cell functions is disrupted, optionally wherein the endogenous gene involved in persistence, expansion, activity, resistance to exhaustion/senescence/inhibitory signals, homing capacity, or other T-cell functions is selected from the group consisting of PD1, TIM3, LAG3, 2B4, KLRG1, TGFbR, CD160, TIGIT, CTLA4 and CD39.
34. A method of preparing a cell, which comprises the step of introducing a polynucleotide according to any of paras 20-22 or a vector according any of paras 23-25 into a cell in vitro, ex vivo or in vivo, for example by transfection or transduction.
35. The method according to para 34, wherein the method comprises the step of T-cell editing, which comprises disrupting an endogenous gene encoding a TCR α chain and/or an endogenous gene encoding a TCR β chain with an artificial nuclease, preferably wherein the artificial nuclease is selected from the group consisting of zinc finger nucleases (ZFNs), transcription activator-like effector nucleases (TALENs) and CRISPR/Cas systems.
36. The method according to para 35, wherein the method comprises the step of targeted integration of an expression cassette into the endogenous gene encoding the TCR α chain and/or the endogenous gene encoding the TCR β chain disrupted by the artificial nuclease, wherein the expression cassette comprises a polynucleotide sequence encoding the TCR of any of paras 1-19.
37. The method according to any of paras 34 to 36, wherein the method comprises the step of disrupting one or more endogenous genes encoding an MHC, optionally wherein the cell prepared by the method is a non-alloreactive universal T-cell.
38. The method according to any of paras 34 to 37, wherein the method comprises the step of disrupting one or more endogenous genes to modify the persistence, expansion, activity, resistance to exhaustion/senescence/inhibitory signals, homing capacity, or other T-cell functions, optionally wherein the method comprises the step of targeted integration of an expression cassette into an endogenous gene involved in persistence, expansion, activity, resistance to exhaustion/senescence/inhibitory signals, homing capacity, or other T-cell functions disrupted by an artificial nuclease, wherein the expression cassette comprises a polynucleotide sequence encoding the TCR of any of paras 1-19, optionally wherein the endogenous gene is selected from the group consisting of PD1, TIM3, LAG3, 2B4, KLRG1, TGFbR, CD160, TIGIT, CTLA4 and CD39.
39. A chimeric molecule comprising the TCR of any of paras 1-19, or a portion thereof, conjugated to a non-cellular substrate, a toxin and/or an antibody, optionally wherein the non-cellular substrate is selected from the group consisting of nanoparticles, exosomes and other non-cellular substrates.
40. A pharmaceutical composition comprising a TCR according to any of paras 1-19, a polynucleotide according to any of paras 20-22, a vector according to any of paras 23-25, a cell according to any of paras 26-33, a cell prepared by a method according to any of paras 34-38, or a chimeric molecule according to para 39.
41. A cell according to any of paras 26-33 or a cell prepared by the method of any of paras 34-38 for use in adoptive cell transfer, preferably adoptive T-cell transfer, optionally wherein the adoptive T-cell transfer is allogenic adoptive T-cell transfer, autologous adoptive T-cell transfer, or universal non-alloreactive adoptive T-cell transfer.
42. A TCR according to any of paras 1-19, a polynucleotide according to any of paras 20-22, a vector according to any of paras 23-25, a cell according to any of paras 26-33, a cell prepared by a method according to any of paras 34-38, a chimeric molecule according to para 39, or a pharmaceutical composition according to para 40 for use in therapy.
43. A TCR according to any of paras 1-19, a polynucleotide according to any of paras 20-22, a vector according to any of paras 23-25, a cell according to any of paras 26-33, a cell prepared by a method according to any of paras 34-38, a chimeric molecule according to para 39, or a pharmaceutical composition according to para 40 for use in treating and/or preventing a proliferative disorder.
44. The TCR, polynucleotide, vector, cell, chimeric molecule, or pharmaceutical composition for use according to para 43, wherein the proliferative disorder is a hematological malignancy or a solid tumor, optionally wherein the hematological malignancy is selected from the group consisting of acute myeloid leukemia (AML), chronic myeloid leukemia (CML), lymphoblastic leukemia, acute lymphocytic leukemia (ALL), myelodisplastic syndromes, lymphoma, multiple myeloma, non-Hodgkin lymphoma, and Hodgkin lymphoma, optionally wherein the solid tumor is selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, oral cancer, hepatocellular carcinoma, bladder cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.
45. The TCR, polynucleotide, vector, cell, chimeric molecule, or pharmaceutical composition for use according to para 43, wherein the proliferative disorder is acute myeloid leukemia (AML).
46. A method for treating and/or preventing a proliferative disorder, which comprises the step of administering a therapeutically effective amount of the TCR of any of paras 1-19, the polynucleotide of any of paras 20-22, the vector of any of paras 23-25, the cell of any of paras 26-33, a cell prepared by the method of any of paras 34-38, the chimeric molecule of para 39, or the pharmaceutical composition of para 40 to a subject in need thereof.
47. The method according to para 46, wherein the proliferative disorder is a hematological malignancy or a solid tumor, optionally wherein the hematological malignancy is selected from the group consisting of acute myeloid leukemia (AML), chronic myeloid leukemia (CML), lymphoblastic leukemia, acute lymphocytic leukemia (ALL), myelodisplastic syndromes, lymphoma, multiple myeloma, non-Hodgkin lymphoma, and Hodgkin lymphoma, optionally wherein the solid tumor is selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, oral cancer, hepatocellular carcinoma, bladder cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.
48. The method according to para 46, wherein the proliferative disorder is acute myeloid leukemia (AML).
49. Use of the TCR of any of paras 1-19, the polynucleotide of any of paras 20-22, the vector of any of paras 23-25, the cell of any of paras 26-33, a cell prepared by the method of any of paras 34-38, the chimeric molecule of para 39, or the pharmaceutical composition of para 40 in the manufacture of a medicament for the treatment and/or prevention of a proliferative disorder.
50. The use according to para 49, wherein the proliferative disorder is a hematological malignancy or a solid tumor, optionally wherein the hematological malignancy is selected from the group consisting of acute myeloid leukemia (AML), chronic myeloid leukemia (CML), lymphoblastic leukemia, acute lymphocytic leukemia (ALL), myelodisplastic syndromes, lymphoma, multiple myeloma, non-Hodgkin lymphoma, and Hodgkin lymphoma, optionally wherein the solid tumor is selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, oral cancer, hepatocellular carcinoma, bladder cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.
51. The use according to para 49, wherein the proliferative disorder is acute myeloid leukemia (AML).

1. A T-cell receptor (TCR) which binds to a Human Telomerase Reverse Transcriptase (hTERT) peptide when presented by a major histocompatibility complex (MHC), wherein the hTERT peptide comprises or consists of the amino acid sequence of SVLNYERARR (SEQ ID NO: 5).

2. The TCR according to claim 1, wherein the TCR comprises:
(i) a CDR3α comprising the amino acid sequence of CADWVDMRF (SEQ ID NO: 8) or a variant thereof having up to three amino acid substitutions, additions or deletions, and a CDR3β comprising the amino acid sequence of CSAPLDRGSNQPQHF (SEQ ID NO: 13) or a variant thereof having up to three amino acid substitutions, additions or deletions;
(ii) a CDR3α comprising the amino acid sequence of CAVSRPNSGYSTLTF (SEQ ID NO: 19) or a variant thereof having up to three amino acid substitutions, additions or deletions, and a CDR3β comprising the amino acid sequence of CASSVRTPSGQETQYF (SEQ ID NO: 24) or a variant thereof having up to three amino acid substitutions, additions or deletions;
(iii) a CDR3α comprising the amino acid sequence of CAVETGGGATNKLIF (SEQ ID NO: 30) or a variant thereof having up to three amino acid substitutions, additions or deletions, and a CDR3β comprising the amino acid sequence of CASSEFWLTQETQYF (SEQ ID NO: 35) or a variant thereof having up to three amino acid substitutions, additions or deletions; or
(iv) a CDR3α comprising the amino acid sequence of CAVSHGRGGATNKLIF (SEQ ID NO: 41) or a variant thereof having up to three amino acid substitutions, additions or deletions, and a CDR3β comprising the amino acid sequence of CASDRVLGYEQYF (SEQ ID NO: 46) or a variant thereof having up to three amino acid substitutions, additions or deletions.

3. The TCR according to claim 1 or 2, wherein the TCR comprises the following CDR sequences:
(i) CDR1α—DSASNY (SEQ ID NO: 6), CDR2α—IRSNVGE (SEQ ID NO: 7), CDR3α—CADWVDMRF (SEQ ID NO: 8), CDR1β—DFQATT (SEQ ID NO: 11), CDR2β—SNEGSKA (SEQ ID NO: 12), and CDR3β—CSAPLDRGSNQPQHF (SEQ ID NO: 13), or variants thereof each having up to three amino acid substitutions, additions or deletions;
(ii) CDR1α—TSGFNG (SEQ ID NO: 17), CDR2α—NVLDGL (SEQ ID NO: 18), CDR3α—CAVSRPNSGYSTLTF (SEQ ID NO: 19), CDR1β—PRHDT (SEQ ID NO: 22), CDR2β—FYEKMQ (SEQ ID NO: 23), and CDR3β—CASSVRTPSGQETQYF (SEQ ID NO: 24), or variants thereof each having up to three amino acid substitutions, additions or deletions;
(iii) CDR1α—VSNAYN (SEQ ID NO: 28), CDR2α—GSKP (SEQ ID NO: 29), CDR3α—CAVETGGGATNKLIF (SEQ ID NO: 30), CDR1β—MNHNS (SEQ ID NO: 33), CDR2β—SASEGT (SEQ ID NO: 34), and CDR3β—CASSEFWLTQETQYF (SEQ ID NO: 35), or variants thereof each having up to three amino acid substitutions, additions or deletions; or
(iv) CDR1α—DSVNN (SEQ ID NO: 39), CDR2α—IPSGT (SEQ ID NO: 40), CDR3α—CAVSHGRGGATNKLIF (SEQ ID NO: 41), CDR1β—MNHNS (SEQ ID NO: 44), CDR2β—SASEGT (SEQ ID NO: 45), and CDR3β—CASDRVLGYEQYF (SEQ ID NO: 46), or variants thereof each having up to three amino acid substitutions, additions or deletions.

4. The TCR according to any of claims 1 to 3, wherein the TCR comprises:
(i) an α chain variable domain comprising the amino acid sequence of SEQ ID NO: 9 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 14 or a variant thereof having at least 70%, at least 75%, at least 80%, at least 85%, at least 90%, at least 95%, at least 98%, or at least 99% sequence identity thereto;
(ii) an α chain variable domain comprising the amino acid sequence of SEQ ID NO: 20 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 25 or a variant thereof having at least 70%, at least 75%, at least 80%, at least 85%, at least 90%, at least 95%, at least 98%, or at least 99% sequence identity thereto;
(iii) an α chain variable domain comprising the amino acid sequence of SEQ ID NO: 31 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 36 or a variant thereof having at least 70%, at least 75%, at least 80%, at least 85%, at least 90%, at least 95%, at least 98%, or at least 99% sequence identity thereto; or
(iv) an α chain variable domain comprising the amino acid sequence of SEQ ID NO: 42 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 47 or a variant thereof having at least 70%, at least 75%, at least 80%, at least 85%, at least 90%, at least 95%, at least 98%, or at least 99% sequence identity thereto.

5. The TCR according to any of claims 1 to 4, wherein the TCR comprises:
(i) an α chain comprising the amino acid sequence of SEQ ID NO: 10 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 15 or 16, or variants thereof having at least 70%, at least 75%, at least 80%, at least 85%, at least 90%, at least 95%, at least 98%, or at least 99% sequence identity thereto;
(ii) an α chain comprising the amino acid sequence of SEQ ID NO: 21 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 26 or 27, or variants thereof having at least 70%, at least 75%, at least 80%, at least 85%, at least 90%, at least 95%, at least 98%, or at least 99% sequence identity thereto;
(iii) an α chain comprising the amino acid sequence of SEQ ID NO: 32 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 37 or 38, or variants thereof having at least 70%, at least 75%, at least 80%, at least 85%, at least 90%, at least 95%, at least 98%, or at least 99% sequence identity thereto; or
(iv) an α chain comprising the amino acid sequence of SEQ ID NO: 43 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 48 or 49, or variants thereof having at least 70%, at least 75%, at least 80%, at least 85%, at least 90%, at least 95%, at least 98%, or at least 99% sequence identity thereto.

6. The TCR according to any of claims 1 to 5, wherein the TCR comprises:
(i) an α chain comprising or consisting of the amino acid sequence of SEQ ID NO: 10; and a β chain comprising or consisting of the amino acid sequence of SEQ ID NO: 15 or 16;
(ii) an α chain comprising or consisting of the amino acid sequence of SEQ ID NO: 21; and a β chain comprising or consisting of the amino acid sequence of SEQ ID NO: 26 or 27;
(iii) an α chain comprising or consisting of the amino acid sequence of SEQ ID NO: 32; and a β chain comprising or consisting of the amino acid sequence of SEQ ID NO: 37 or 38; or
(iv) an α chain comprising or consisting of the amino acid sequence of SEQ ID NO: 43; and a β chain comprising or consisting of the amino acid sequence of SEQ ID NO: 48 or 49.

7. The TCR according to any of claims 1 to 6, wherein the TCR is restricted to HLA-A*0301.

8. A T-cell receptor (TCR) which binds to Survivin peptide when presented by a major histocompatibility complex (MHC), wherein the TCR comprises a CDR3α comprising the amino acid sequence of CALDRMDSSYKLIF (SEQ ID NO: 54) or a variant thereof having up to three amino acid substitutions, additions or deletions, and a CDR3β comprising the amino acid sequence of CASSYDODGEAFF (SEQ ID NO: 59) or a variant thereof having up to three amino acid substitutions, additions or deletions.

9. The TCR according to claim 8, wherein the TCR comprises the following CDR sequences: CDR1α—NYSPAY (SEQ ID NO: 52), CDR2α—IRENEKE (SEQ ID NO: 53), CDR3α—CALDRMDSSYKLIF (SEQ ID NO: 54), CDR1β—MNHEY (SEQ ID NO: 57), CDR2β—SVGAGI (SEQ ID NO: 58), and CDR3β—CASSYDQDGEAFF (SEQ ID NO: 59), or variants thereof each having up to three amino acid substitutions, additions or deletions.

10. The TCR according to claim 8 or 9, wherein the TCR comprises the following CDR sequences: CDR1α—NYSPAY (SEQ ID NO: 52), CDR2α—IRENEKE (SEQ ID NO: 53), CDR3α—CALDRMDSSYKLIF (SEQ ID NO: 54), CDR1β—MNHEY (SEQ ID NO: 57), CDR2β—SVGAGI (SEQ ID NO: 58), and CDR3β—CASSYDQDGEAFF (SEQ ID NO: 59).

11. The TCR according to any of claims 8-10, wherein the TCR comprises an α chain variable domain comprising the amino acid sequence of SEQ ID NO: 55 or a variant thereof having at least 70%, at least 75%, at least 80%, at least 85%, at least 90%, at least 95%, at least 98%, or at least 99% sequence identity thereto; and a β chain variable domain comprising the amino acid sequence of SEQ ID NO: 60 or a variant thereof having at least 70%, at least 75%, at least 80%, at least 85%, at least 90%, at least 95%, at least 98%, or at least 99% sequence identity thereto.

12. The TCR according to any of claims 8-11, wherein the TCR comprises an α chain comprising the amino acid sequence of SEQ ID NO: 56 or a variant thereof having at least 70%, at least 75%, at least 80%, at least 85%, at least 90%, at least 95%, at least 98%, or at least 99% sequence identity thereto; and a β chain comprising the amino acid sequence of SEQ ID NO: 61 or 62, or variants thereof having at least 70%, at least 75%, at least 80%, at least 85%, at least 90%, at least 95%, at least 98%, or at least 99% sequence identity thereto.

13. The TCR according to any of claims 8-12, wherein the TCR comprises an α chain comprising or consisting of the amino acid sequence of SEQ ID NO: 56; and a β chain comprising or consisting of the amino acid sequence of SEQ ID NO: 61 or 62.

14. The TCR according to any of claims 8-13, wherein the TCR is restricted to HLA-A*0201.

15. The TCR according to any of claims 8-14, wherein the Survivin peptide comprises or consists of the amino acid sequence of LMLGEFLKL (SEQ ID NO: 51) or a variant thereof having up to three amino acid substitutions, additions or deletions.

16. The TCR according to any preceding claim, wherein the TCR comprises one or more mutations at the α chain/β chain interface, such that when the α chain and the β chain are expressed in a T-cell, the frequency of mispairing between said chains and endogenous TCR α and β chains is reduced.

17. The TCR according to claim 16, wherein the one or more mutations introduce a cysteine residue into the constant region domain of each of the α chain and the β chain, wherein the cysteine residues are capable of forming a disulphide bond between the α chain and the β chain.

18. The TCR according to any preceding claim, wherein the TCR comprises a murinised constant region.

19. The TCR according to any preceding claim, wherein the TCR is a soluble TCR.

20. A polynucleotide encoding the α chain and/or the β chain of a TCR according to any preceding claim.

21. The polynucleotide according to claim 20, wherein the polynucleotide encodes the α chain linked to the β chain.

22. The polynucleotide according to claim 20 or 21, wherein the polynucleotide further encodes one or more short interfering RNA (siRNA) or other agents capable of reducing or preventing expression of one or more endogenous TCR genes.

23. A vector comprising a polynucleotide according to any of claims 20-22.

24. The vector according to claim 23, wherein the vector is a plasmid or a viral vector.

25. The vector according to claim 23 or 24, wherein the vector comprises a polynucleotide which encodes one or more CD3 chains, CD8, a suicide gene and/or a selectable marker.

26. A cell comprising a TCR according to any of claims 1-19, a polynucleotide according to any of claims 20-22 or a vector according to any of claims 23-25.

27. The cell according to claim 26, wherein the cell further comprises a vector which encodes one or more CD3 chains, CD8, a suicide gene and/or a selectable marker.

28. The cell according to claim 26 or 27, wherein the cell is a T-cell, a lymphocyte, or a stem cell.

29. The cell according to claim 28, wherein the T-cell, the lymphocyte, or the stem cell is selected from the group consisting of CD4+ cells, CD8+ cells, naive T-cells, memory stem T-cells, central memory T-cells, double negative T-cells, effector memory T-cells, effector T-cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, cytokine-induced killer (CIK) cells, hematopoietic stem cells and pluripotent stem cells.

30. The cell according to any of claims 26 to 29, wherein the cell is a T-cell, optionally wherein the cell is a T-cell which has been isolated from a subject.

31. The cell according to any of claims 26 to 30, wherein an endogenous gene encoding a TCR α chain and/or an endogenous gene encoding a TCR β chain is disrupted, preferably such that the endogenous gene encoding a TCR α chain and/or the endogenous gene encoding a TCR β chain is not expressed, optionally wherein the endogenous gene encoding a TCR α chain and/or the endogenous gene encoding a TCR β chain is disrupted by insertion of an expression cassette comprising a polynucleotide sequence encoding the TCR of any of claims 1-19.

32. The cell according to any of claims 26 to 31, wherein one or more endogenous genes encoding an MHC is disrupted.

33. The cell according to any of claims 26 to 32, wherein an endogenous gene involved in persistence, expansion, activity, resistance to exhaustion/senescence/inhibitory signals, homing capacity, or other T-cell functions is disrupted, optionally wherein the endogenous expansion, gene involved in persistence, activity, resistance to exhaustion/senescence/inhibitory signals, homing capacity, or other T-cell functions is selected from the group consisting of PD1, TIM3, LAG3, 2B4, KLRG1, TGFbR, CD160, TIGIT, CTLA4 and CD39.

34. A method of preparing a cell, which comprises the step of introducing a polynucleotide according to any of claims 20-22 or a vector according any of claims 23-25 into a cell in vitro, ex vivo or in vivo, for example by transfection or transduction.

35. The method according to claim 34, wherein the method comprises the step of T-cell editing, which comprises disrupting an endogenous gene encoding a TCR α chain and/or an endogenous gene encoding a TCR β chain with an artificial nuclease, preferably wherein the artificial nuclease is selected from the group consisting of zinc finger nucleases (ZFNs), transcription activator-like effector nucleases (TALENs) and CRISPR/Cas systems.

36. The method according to claim 35, wherein the method comprises the step of targeted integration of an expression cassette into the endogenous gene encoding the TCR α chain and/or the endogenous gene encoding the TCR β chain disrupted by the artificial nuclease, wherein the expression cassette comprises a polynucleotide sequence encoding the TCR of any of claims 1-19.

37. The method according to any of claims 34 to 36, wherein the method comprises the step of disrupting one or more endogenous genes encoding an MHC, optionally wherein the cell prepared by the method is a non-alloreactive universal T-cell.

38. The method according to any of claims 34 to 37, wherein the method comprises the step of disrupting one or more endogenous genes to modify the persistence, expansion, activity, resistance to exhaustion/senescence/inhibitory signals, homing capacity, or other T-cell functions, optionally wherein the method comprises the step of targeted integration of an expression cassette into an endogenous gene involved in persistence, expansion, activity, resistance to exhaustion/senescence/inhibitory signals, homing capacity, or other T-cell functions disrupted by an artificial nuclease, wherein the expression cassette comprises a polynucleotide sequence encoding the TCR of any of claims 1-19, optionally wherein the endogenous gene is selected from the group consisting of PD1, TIM3, LAG3, 2B4, KLRG1, TGFbR, CD160, TIGIT, CTLA4 and CD39.

39. A chimeric molecule comprising the TCR of any of claims 1-19, or a portion thereof, conjugated to a non-cellular substrate, a toxin and/or an antibody, optionally wherein the non-cellular substrate is selected from the group consisting of nanoparticles, exosomes and other non-cellular substrates.

40. A pharmaceutical composition comprising a TCR according to any of claims 1-19, a polynucleotide according to any of claims 20-22, a vector according to any of claims 23-25, a cell according to any of claims 26-33, a cell prepared by a method according to any of claims 34-38, or a chimeric molecule according to claim 39.

41. A cell according to any of claims 26-33 or a cell prepared by the method of any of claims 34-38 for use in adoptive cell transfer, preferably adoptive T-cell transfer, optionally wherein the adoptive T-cell transfer is allogenic adoptive T-cell transfer, autologous adoptive T-cell transfer, or universal non-alloreactive adoptive T-cell transfer.

42. A TCR according to any of claims 1-19, a polynucleotide according to any of claims 20-22, a vector according to any of claims 23-25, a cell according to any of claims 26-33, a cell prepared by a method according to any of claims 34-38, a chimeric molecule according to claim 39, or a pharmaceutical composition according to claim 40 for use in therapy.

43. A TCR according to any of claims 1-19, a polynucleotide according to any of claims 20-22, a vector according to any of claims 23-25, a cell according to any of claims 26-33, a cell prepared by a method according to any of claims 34-38, a chimeric molecule according to claim 39, or a pharmaceutical composition according to claim 40 for use in treating and/or preventing a proliferative disorder.

44. The TCR, polynucleotide, vector, cell, chimeric molecule, or pharmaceutical composition for use according to claim 43, wherein the proliferative disorder is a hematological malignancy or a solid tumor, optionally wherein the hematological malignancy is selected from the group consisting of acute myeloid leukemia (AML), chronic myeloid leukemia (CML), lymphoblastic leukemia, acute lymphocytic leukemia (ALL), myelodisplastic syndromes, lymphoma, multiple myeloma, non-Hodgkin lymphoma, and Hodgkin lymphoma, optionally wherein the solid tumor is selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, oral cancer, hepatocellular carcinoma, bladder cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.

45. The TCR, polynucleotide, vector, cell, chimeric molecule, or pharmaceutical composition for use according to claim 43, wherein the proliferative disorder is acute myeloid leukemia (AML).

46. A method for treating and/or preventing a proliferative disorder, which comprises the step of administering a therapeutically effective amount of the TCR of any of claims 1-19, the polynucleotide of any of claims 20-22, the vector of any of claims 23-25, the cell of any of claims 26-33, a cell prepared by the method of any of claims 34-38, the chimeric molecule of claim 39, or the pharmaceutical composition of claim 40 to a subject in need thereof.

47. The method according to claim 46, wherein the proliferative disorder is a hematological malignancy or a solid tumor, optionally wherein the hematological malignancy is selected from the group consisting of acute myeloid leukemia (AML), chronic myeloid leukemia (CML), lymphoblastic leukemia, acute lymphocytic leukemia (ALL), myelodisplastic syndromes, lymphoma, multiple myeloma, non-Hodgkin lymphoma, and Hodgkin lymphoma, optionally wherein the solid tumor is selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, oral cancer, hepatocellular carcinoma, bladder cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.

48. The method according to claim 46, wherein the proliferative disorder is acute myeloid leukemia (AML).

49. Use of the TCR of any of claims 1-19, the polynucleotide of any of claims 20-22, the vector of any of claims 23-25, the cell of any of claims 26-33, a cell prepared by the method of any of claims 34-38, the chimeric molecule of claim 39, or the pharmaceutical composition of claim 40 in the manufacture of a medicament for the treatment and/or prevention of a proliferative disorder.

50. The use according to claim 49, wherein the proliferative disorder is a hematological malignancy or a solid tumor, optionally wherein the hematological malignancy is selected from the group consisting of acute myeloid leukemia (AML), chronic myeloid leukemia (CML), lymphoblastic leukemia, acute lymphocytic leukemia (ALL), myelodisplastic syndromes, lymphoma, multiple myeloma, non-Hodgkin lymphoma, and Hodgkin lymphoma, optionally wherein the solid tumor is selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, oral cancer, hepatocellular carcinoma, bladder cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.

51. The use according to claim 49, wherein the proliferative disorder is acute myeloid leukemia (AML).
